SEQUENCE LISTING (46 sequences)

SEQ ID NO: 1 - DNA, 30 bp, synthetic construct
  source 1..30: mol_type = other DNA
  misc_difference order(1..3,27..29): note = Phosphorothioate
aaaaaaaaaaaaaaaaaaaaaaaaaaaaaa

SEQ ID NO: 2 - DNA, 30 bp, synthetic construct
  source 1..30: mol_type = other DNA
  misc_difference order(1..3,27..29): note = Phosphorothioate
tttttttttttttttttttttttttttttt

SEQ ID NO: 3 - DNA, 50 bp, synthetic construct
  source 1..50: mol_type = other DNA
  misc_difference order(1..3,47..49): note = Phosphorothioate
aaaaaaaaaaaaaaaaaaaaaaaaaaaaaaaaaaaaaaaaaaaaaaaaaa

SEQ ID NO: 4 - DNA, 50 bp, synthetic construct
  source 1..50: mol_type = other DNA
  misc_difference order(1..3,47..49)
tttttttttttttttttttttttttttttttttttttttttttttttttt

SEQ ID NO: 5 - DNA, 60 bp, synthetic construct
  source 1..60: mol_type = other DNA
  misc_difference order(1..3,57..59): note = Phosphorothioate
aaaaaaaaaaaaaaaaaaaaaaaaaaaaaaaaaaaaaaaaaaaaaaaaaaaaaaaaaaaa

SEQ ID NO: 6 - DNA, 60 bp, synthetic construct
  source 1..60: mol_type = other DNA
  misc_difference order(1..3,57..59): note = Phosphorothioate
tttttttttttttttttttttttttttttttttttttttttttttttttttttttttttt

SEQ ID NO: 7 - DNA, 70 bp, synthetic construct
  source 1..70: mol_type = other DNA
  misc_difference order(1..3,67..69): note = Phosphorothioate
aaaaaaaaaaaaaaaaaaaaaaaaaaaaaaaaaaaaaaaaaaaaaaaaaaaaaaaaaaaaaaaaaaaaaa

SEQ ID NO: 8 - DNA, 70 bp, synthetic construct
  source 1..70: mol_type = other DNA
  misc_difference order(1..3,67..69): note = Phosphorothioate
tttttttttttttttttttttttttttttttttttttttttttttttttttttttttttttttttttttt

SEQ ID NO: 9 - DNA, 80 bp, synthetic construct
  source 1..80: mol_type = other DNA
  misc_difference order(1..3,77..79): note = Phosphorothioate
aaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 10 - DNA, 80 bp, synthetic construct
  source 1..80: mol_type = other DNA
  misc_difference order(1..3,77..79): note = Phosphorothioate
tttttttttttttttttttttttttttttttttttttttttttttttttttttttttttttttttttttttttttttttt

SEQ ID NO: 11 - DNA, 90 bp, synthetic construct
  source 1..90: mol_type = other DNA
  misc_difference order(1..3,87..89): note = Phosphorothioate
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 12 - DNA, 90 bp, synthetic construct
  source 1..90: mol_type = other DNA
  misc_difference order(1..3,87..89): note = Phosphorothioate
tttttttttttttttttttttttttttttttttttttttttttttttttttttttttttttttttttttttttttttttttttttttttt

SEQ ID NO: 13 - DNA, 100 bp, synthetic construct
  source 1..100: mol_type = other DNA
  misc_difference order(1..3,97..99): note = Phosphorothioate
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 14 - DNA, 100 bp, synthetic construct
  source 1..100: mol_type = other DNA
  misc_difference order(1..3,97..99): note = Phosphorothioate
tttttttttttttttttttttttttttttttttttttttttttttttttttttttttttttttttttttttttttttttttttttttttttttttttttt

SEQ ID NO: 15 - DNA, 110 bp, synthetic construct
  source 1..110: mol_type = other DNA
  misc_difference order(1..3,107..109): note = Phosphorothioate
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 16 - DNA, 110 bp, synthetic construct
  source 1..110: mol_type = other DNA
  misc_difference order(1..3,107..109): note = Phosphorothioate
tttttttttttttttttttttttttttttttttttttttttttttttttttttttttttttttttttttttttttttttttttttttttttttttttttttttttttttt

SEQ ID NO: 17 - DNA, 30 bp, synthetic construct
  source 1..30: mol_type = other DNA
  misc_difference order(1..3,27..29): note = Phosphorothioate
gcgcgcgcgcgcgcgcgcgcgcgcgcgcgc

SEQ ID NO: 18 - DNA, 50 bp, synthetic construct
  source 1..50: mol_type = other DNA
  misc_difference order(1..3,47..49): note = Phosphorothioate
gcgcgcgcgcgcgcgcgcgcgcgcgcgcgcgcgcgcgcgcgcgcgcgcgc

SEQ ID NO: 19 - DNA, 60 bp, synthetic construct
  source 1..60: mol_type = other DNA
  misc_difference order(1..3,57..59): note = Phosphorothioate
gcgcgcgcgcgcgcgcgcgcgcgcgcgcgcgcgcgcgcgcgcgcgcgcgcgcgcgcgcgc

SEQ ID NO: 20 - DNA, 70 bp, synthetic construct
  source 1..70: mol_type = other DNA
  misc_difference order(1..3,67..69): note = Phosphorothioate
gcgcgcgcgcgcgcgcgcgcgcgcgcgcgcgcgcgcgcgcgcgcgcgcgcgcgcgcgcgcgcgcgcgcgc

SEQ ID NO: 21 - DNA, 80 bp, synthetic construct
  source 1..80: mol_type = other DNA
  misc_difference order(1..3,17..19): note = Phosphorothioate
gcgcgcgcgcgcgcgcgcgcgcgcgcgcgcgcgcgcgcgcgcgcgcgcgcgcgcgcgcgcgcgcgcgcgcgcgcgcgcgc

SEQ ID NO: 22 - DNA, 90 bp, synthetic construct
  source 1..90: mol_type = other DNA
  misc_difference order(1..3,87..89): note = Phosphorothioate
gcgcgcgcgcgcgcgcgcgcgcgcgcgcgcgcgcgcgcgcgcgcgcgcgcgcgcgcgcgcgcgcgcgcgcgcgcgcgcgcgcgcgcgcgc

SEQ ID NO: 23 - DNA, 100 bp, synthetic construct
  source 1..100: mol_type = other DNA
  misc_difference order(1..3,97..99): note = Phosphorothioate
gcgcgcgcgcgcgcgcgcgcgcgcgcgcgcgcgcgcgcgcgcgcgcgcgcgcgcgcgcgcgcgcgcgcgcgcgcgcgcgcgcgcgcgcgcgcgcgcgcgc

SEQ ID NO: 24 - DNA, 76 bp, synthetic construct
  source 1..76: mol_type = other DNA
  misc_difference order(1..3,73..75): note = Phosphorothioate
aaaaaaaaaaaaaaaaaaaaaaaaaaaaaaaaaaaaaaaaaaaaaaaaaaaaaaaaaaaaaaaaaaaaaaaaaaaa

SEQ ID NO: 25 - DNA, 76 bp, synthetic construct
  source 1..76: mol_type = other DNA
  misc_difference order(1..3,73..75): note = Phosphorothioate
tttttttttttttttttttttttttttttttttttttttttttttttttttttttttttttttttttttttttttt

SEQ ID NO: 26 - DNA, 76 bp, synthetic construct
  source 1..76: mol_type = other DNA
  misc_difference order(1..3,73..75): note = Phosphorothioate
acacacacacacacacacacacacacacacacacacacacacacacacacacacacacacacacacacacacacac

SEQ ID NO: 27 - DNA, 76 bp, synthetic construct
  source 1..76: mol_type = other DNA
  misc_difference order(1..3,73..75): note = Phosphorothioate
gtgtgtgtgtgtgtgtgtgtgtgtgtgtgtgtgtgtgtgtgtgtgtgtgtgtgtgtgtgtgtgtgtgtgtgtgtgt

SEQ ID NO: 28 - DNA, 76 bp, synthetic construct
  source 1..76: mol_type = other DNA
  misc_difference order(1..3,73..75): note = Phosphorothioate
atatatatatatatatatatatatatatatatatatatatatatatatatatatatatatatatatatatatatat

SEQ ID NO: 29 - DNA, 76 bp, synthetic construct
  source 1..76: mol_type = other DNA
  misc_difference order(1..3,73..75): note = Phosphorothioate
tatatatatatatatatatatatatatatatatatatatatatatatatatatatatatatatatatatatatata

SEQ ID NO: 30 - DNA, 76 bp, synthetic construct
  source 1..76: mol_type = other DNA
  misc_difference order(1..3,73..75): note = Phosphorothioate
actgactgactgactgactgactgactgactgactgactgactgactgactgactgactgactgactgactgactg

SEQ ID NO: 31 - DNA, 76 bp, synthetic construct
  source 1..76: mol_type = other DNA
  misc_difference order(1..3,73..75): note = Phosphorothioate
cagtcagtcagtcagtcagtcagtcagtcagtcagtcagtcagtcagtcagtcagtcagtcagtcagtcagtcagt

SEQ ID NO: 32 - DNA, 76 bp, synthetic construct
  source 1..76: mol_type = other DNA
  misc_difference order(1..3,73..75): note = Phosphorothioate
gaccctatcgatacagggcacggggtcgaactgttgggtttcgccatggtaccccctgcatttatatagccagacc

SEQ ID NO: 33 - DNA, 76 bp, synthetic construct
  source 1..76: mol_type = other DNA
  misc_difference order(1..3,73..75): note = Phosphorothioate
ggtctggctatataaatgcagggggtaccatggcgaaacccaacagttcgaccccgtgccctgtatcgatagggtc

SEQ ID NO: 34 - skipped

SEQ ID NO: 35 - DNA, 90 bp, synthetic construct; identical to SEQ ID NO: 11
  source 1..90: mol_type = other DNA
  misc_difference order(1..3,87..89): note = Phosphorothioate

SEQ ID NO: 36 - DNA, 90 bp, synthetic construct; identical to SEQ ID NO: 12
  source 1..90: mol_type = other DNA
  misc_difference order(1..3,87..89): note = Phosphorothioate

SEQ ID NO: 37 - DNA, 76 bp, synthetic construct
  source 1..76: mol_type = other DNA
  misc_difference order(1..3,73..75): note = Phosphorothioate
attaattaattaattaattaattaattaattaattaattaattaattaattaattaattaattaattaattaatta

SEQ ID NO: 38 - DNA, 76 bp, synthetic construct
  source 1..76: mol_type = other DNA
  misc_difference order(1..3,73..75): note = Phosphorothioate
taattaattaattaattaattaattaattaattaattaattaattaattaattaattaattaattaattaattaat

SEQ ID NO: 39 - DNA, 76 bp, synthetic construct; identical to SEQ ID NO: 30
  source 1..76: mol_type = other DNA
  misc_difference order(1..3,73..75): note = Phosphorothioate

SEQ ID NO: 40 - DNA, 76 bp, synthetic construct; identical to SEQ ID NO: 31
  source 1..76: mol_type = other DNA
  misc_difference order(1..3,73..75): note = Phosphorothioate

SEQ ID NO: 41 - DNA, 76 bp, synthetic construct; identical to SEQ ID NO: 32
  source 1..76: mol_type = other DNA
  misc_difference order(1..3,73..75): note = Phosphorothioate

SEQ ID NO: 42 - DNA, 76 bp, synthetic construct; identical to SEQ ID NO: 33
  source 1..76: mol_type = other DNA
  misc_difference order(1..3,73..75): note = Phosphorothioate

SEQ ID NO: 43 - DNA, 90 bp, synthetic construct; identical to SEQ ID NO: 11
  source 1..90: mol_type = other DNA
  misc_difference order(1..3,87..89): note = Phosphorothioate

SEQ ID NO: 44 - DNA, 90 bp, synthetic construct; identical to SEQ ID NO: 12
  source 1..90: mol_type = other DNA
  misc_difference order(1..3,57..59): note = Phosphorothioate

SEQ ID NO: 45 - DNA, 90 bp, synthetic construct; identical to SEQ ID NO: 22
  source 1..90: mol_type = other DNA

SEQ ID NO: 46 - DNA, 90 bp, synthetic construct
  source 1..90: mol_type = other DNA
  misc_difference order(1..3,87..89): note = Phosphorothioate
cgcgcgcgcgcgcgcgcgcgcgcgcgcgcgcgcgcgcgcgcgcgcgcgcgcgcgcgcgcgcgcgcgcgcgcgcgcgcgcgcgcgcgcgcg

(19) United States
(12) Patent Application Publication
(10) Pub. No.: US 2025/0019772 A1
(43) Pub. Date: Jan. 16, 2025

(54) Method and System of Augmenting Cancer Therapy

(71) Applicant: UNIVERSITY OF MIAMI, Miami, FL (US)

(72) Inventor: Glen N. Barber, Miami, FL (US)

(21) Appl. No.: 18/745,908

(22) Filed: Jun. 17, 2024

Related U.S. Application Data
(60) Provisional application No. 63/521,696, filed on Jun. 18, 2023.

(51) Int. Cl. C12Q 1/6886; A61K 38/21

(52) U.S. Cl. CPC C12Q 1/6886; A61K 38/21; C12Q 2600/158

(57) ABSTRACT

Following DNA damage events, such as radiation therapy, cytosolic dsDNA species are generated that trigger intrinsic STimulator of Interferon Genes (STING) signaling and render cells competent for phagocytic activation contingent on the chemokine CCL5 (C-C Motif Ligand 5). Loss of STING signaling or inducible CCL5, rendered DNA-damaged cells incapable of APC stimulation following phagocytosis. STING signaling facilitated the generation of immunogenic endosomal vesicles comprising the autophagy related proteins RAB9 and LC3, and cytosolic DNA which triggered APC activation in trans, an event which also required extrinsic cGAS/STING-dependent signaling. Further, the combination of radiation treatment in the presence of type I IFN greatly reduced tumor growth while rescuing the ability to generate CTL activity. Reconstitution of STING signaling and/or the combination of radiation treatment in the presence of type I IFN could significantly augment cancer therapy.

### CROSS REFERENCE TO RELATED APPLICATIONS

The present application claims the benefit of priority to U.S. Provisional Application No. 63/521,696, filed Jun. 18, 2024, the disclosure of which is incorporated herein by reference in its entirety.

### FIELD OF THE INVENTION

The present invention relates to methods of augmenting cancer therapy.

### INCORPORATION BY REFERENCE

This application contains, as a separate part of the disclosure, a sequence listing in computer-readable form (filename: 70445_SeqListing.xml, 60,632 bytes, created Oct. 8, 2024), which is incorporated by reference in its entirety.

### BACKGROUND OF THE INVENTION

STimulator of Interferon Genes (STING) is a 379 amino acid transmembrane protein located in the cytosol endoplasmic reticulum of for example fibroblasts, macrophages and dendritic cells (DCs). STING is a DNA sensor that has evolved to detect microbial infection of the cell. STING is activated by cyclic dinucleotides (CDN's) such as cyclic di-GMP and cyclic-di-AMP secreted by intracellular bacteria following infection. Alternatively, STING can be activated by cyclic GMP-AMP (cGAMP) generated by a cellular cGAMP synthase cGAS (MB21D1) after association with aberrant cytosolic dsDNA species, which can include microbial DNA or self-DNA leaked from the nucleus into the cytosol. Cancer therapies such as radiation therapy result in dsDNA leaking from the nucleus to the cytosol. CDN-binding results in STING, complexed with the IRF3 kinase TANK-binding kinase 1 (TBK1) re-locating to perinuclear regions of the cell. Association with CDN's enables STING to activate the transcription factors IRF3 and NF-κB which stimulate the production of type I interferon (IFN) and pro-inflammatory cytokines, which facilitate adaptive immunity.

The elimination of apoptotic cells by phagocytes generally transpires without triggering deleterious inflammatory events that could be harmful to the host1. This process has evolved to trigger the rapid degradation of self-nucleic acid within the dying cell by host DNAses. Failure to degrade genomic DNA from such cells can lead to the inadvertent stimulation of DNA-activated innate immune sensors in engulfing phagocytes, such as those controlled by STING signaling, with lethal pro-inflammatory consequences. Cancer cells are notoriously non-immunogenic in part, since they are phagocytosed comparable to normal apoptotic cells and avoid transiently triggering innate immune pathways required to facilitate tumor cell antigen presentation and the cross-priming of T-cells. It is therefore unclear how antigen presenting cells (APCs) immunologically recognize a DNA-damaged, potentially pre-cancerous cell, to facilitate anti-tumor CTL activity and immune clearance. Generally, the cytosol of the cell is free of dsDNA species, unless introduced following microbial infection or leaked from the nucleus after cellular division or following DNA-damage. Cytosolic dsDNA species associate with the synthase cGAS, to generate cyclic dinucleotides (CDN's) that bind to and trigger intrinsic STING signaling presumably to activate host defense countermeasures, including attracting phagocytes to the microenvironment. However, the exact mechanisms, including the identification of key inducible genes important for this process remain to be identified. Subsequently, efficient APC activation requires extrinsic cGAS/STING-dependent signaling, a process that can also be triggered in trans, by dsDNA species present in the engulfed cell that has escaped complete DNase degradation.

### SUMMARY OF THE INVENTION

In one aspect, the present application relates to a method of treating cancer in a subject in need thereof including administering a radiation therapy to the subject, where the subject has been determined to be responsive to the radiation therapy including determining that the CCL5 gene is expressed in the subject.

In one aspect, the present application relates to a method of treating cancer in a subject in need thereof including administering a radiation therapy to the subject, where the subject has been determined to be responsive to the radiation therapy including determining that the CCL5 gene is expressed in the subject and administering type I interferon to the subject while administering radiation therapy to the subject.

In one aspect, the present application relates to a method of treating cancer in a subject in need thereof including determining that the CCL5 gene is expressed in the subject and administering a radiation therapy to the subject.

In another aspect, the present application relates to a method of treating cancer in a subject in need thereof including determining that the CCL5 gene is expressed in the subject and administering type I interferon to the subject while administering a radiation therapy to the subject.

### BRIEF DESCRIPTION OF THE DRAWINGS

Various embodiments of the present invention will be described in detail based on the following Figures, where:

FIG. 1 shows both intrinsic STING signaling and cytosolic dsDNA species are required for the trans activation of phagocytes and generation of CTL's. (A) Immunoblotting analysis of STING, cGAS, and IRF3 in B16-OVA-WT, B16-OVA-SKO, or B16-OVA-CKO cells treated with 50 Gy X-ray for 3 days. (B) qPCR analysis of CCL5 in cells depicted in (A). (C) Immunofluorescent analysis using anti-double strand DNA in irradiated WT, SKO or CKO B16-OVA cells. Original magnification X200. (D) Quantitation of dsDNA by Picogreen staining in cytoplasmic extracts from WT, SKO and CKO B16-OVA cells treated with 50 Gy X-ray for 3 days. (E) Schematic representation of the phagocytosis of irradiated B16-OVA cells. B16-WT, STING knockout (B16-SKO) or cGAS knockout (B16-CKO) were irradiated by X-ray (50 Gy) and fed to bone marrow derived macrophages lacking STING or cGAS (WTØ, SKOØ, CKOØ) at 72 h following X-ray irradiation, at a ratio of 10:1. (F) Flow cytometry analysis of macrophage engulfment efficiency following the phagocytosis of irradiated CFSE-labeled B16-WT, B16-SKO and B16-CKO cells after 6 h. % of phagocytosis: CSFE+ of CD11b+ cells. (G and H) qPCR analysis of IFNβ1 and Cxcl10 in WTØ, SKOØ, CKOØ APC's following the phagocytosis of irradiated WT, SKO and CKO B16 cells, after 6 h. (I) cGAMP ELISA analysis of B16 WT, SKO and CKO cells at 3days after irradiation at 50 Gy. (J) cGAMP ELISA analysis of CD11c+ BMDC (DCs) following the phagocytosis of irradiated B16 WT, SKO and CKO cells at 50 Gy X-ray. (K) qPCR analysis of IFNb1 in DCs following the phagocytosis of irradiated B16-WT or SKO cells. (L-O) Anti-tumor efficacy and cytotoxic T cell responses following radiation therapy in C57/BL6 WT mice. 5×10⁵ of B16 WT, SKO and CKO cells were inoculated into right flank of WT CL57/B6 mice (n=7/group). 7 days after inoculation, tumors were locally exposed to X-rays at 8 Gy, three times consecutively. (L) Tumor volume was measured every other day for 2 weeks. (M) Abscopal challenge with B16-WT cells on the other side of flanks in the irradiated mice harboring B16-WT. B16-SKO, and B16 CKO cells in (F). Naïve mice were used as a control group. Tumor volumes were monitored every other day for 2 weeks. (N) IFNγ ELISPOT analysis of OVA antigen in CD8+ T cells isolated from the same mice as in (F). (O) Immunohistochemistry analysis of F4/80 and CD11c in tumors retrieved from the same mice in (F). Data is representative of at least three independent experiments. Error bars indicate mean±SD. *p<0.05; Student's t-test, according to various embodiments of the present invention;

FIG. 2 shows reconstituting STING/cGAS signaling rescues DNA-damage mediated bystander immunogenicity in vivo. (A-E) Murine AML C1498 cells were stably reconstituted with pcDNA-cGAS (C1498-cGAS) or empty vector (C1498-Vec). The cells were treated by X-ray radiation at 50 Gy. (A) Immunoblot blot analysis of STING, cGAS, TBK1, and IRF3 in C1498-Vec or C1498-cGAS transfected with 3 μg/mL of ISD for 6 h. (B) qPCR analysis of IFNβ1 and Cxcl10 in the same cells depicted in (A). (C) Immunoblot analysis of STING, cGAS, TBK1, and IRF3 in C1498-Vec or C1498-cGAS irradiated by X-ray at 5 Gy for the indicated times. (D) qPCR analysis of IFNβ1 and Cxcl10 in the same cells depicted in (C). (E) qPCR analysis of IFNβ1 and Cxcl10 in WTØ and SKOØ APC's following the phagocytosis of C1498 and C1498-cGAS after 6 h. (F) 1×10⁶ C1498 or C1498-cGAS cells were inoculated into the right flank of WT CL57/B6 mice (n=7/group). 7 days after inoculation, tumors were locally exposed to X-rays at 8 Gy three times consecutively (left). Abscopal challenge with C1498 cells 5 days after X-ray treatment on the alternate flanks of the same mice (right). Primary tumors and Abscopal tumors were measured every other day for 17 days. (G) Immunoblotting analysis of STING, cGAS, TBK1 and IRF3, and (H) qPCR analysis of IFNβ1 and Cxcl10 in C1498 cells treated with or without 5-Azadc at 1 μM for 3 days, followed by transfection with ISD for 6 h. (I) Immunoblotting analysis of STING, cGAS, TBK1 and IRF3, and (J) qPCR analysis of IFNβ1 and Cxcl10 in C1498 cells treated with or without 5-Azadc at 1 μM for 3 days, followed by X-ray treatment at 5 Gy for the indicated times. (K) qPCR analysis of IFNβ1 and Cxcl10 in WTØ and SKOØ APC's following the phagocytosis of C1498 cells treated with or without 5-Azadc at 1 μM for 3 days, followed by X-ray treatment at 5 Gy for 3 days. (L) Schematic of the in vivo recapitulation of cGAS in WT or SKO C1498 tumors. C57/BL6 mice were subcutaneously injected with 1×10⁶ of WT or C1498-SKO C1498 cells on the flanks (n=7/group). 10 days after inoculation, tumors were locally irradiated by 8 Gy X-rays, three times consecutively and 5-Azade was intraperitoneally injected five times every 2 days. (M) Tumor volumes were measured every other day in mice bearing C1498-WT or C1498-SKO cells. (N) Abscopal challenge with C1498 cells 5 days after X-ray irradiation, on the alternate flanks of mice bearing C1498 and C1498-SKO cells, in presence or absence of 5-Azadc. Abscopal tumors were measured every other day for 17 days. Data is representative of at least three independent experiments. Error bars indicate mean±SD. *p<0.05; Student's t-test, according to various embodiments of the present invention;

FIG. 3 shows STING-induced CCL5 is required for DNA-damaged mediated immunity. (A) Gene array analysis of WT, SKO, CKO and IKO MEFs irradiated with X-rays at 50 Gy for 3 days. Total RNA was purified, and transcripts were analyzed by Affymetrix microarray. Highly induced genes were only evident in irradiated WT cells. (B) qPCR analysis of IFNβ1 and Cxcl10 in WTØ and SKOØ APC's following the phagocytosis of MEF-WT, MEF-SKO, MEF-CCL5 KO cells irradiated by X-rays at 50 Gy. (C) qPCR analysis of IFNβ1 and Cxcl10 in WTØ and SKOØ APC's following the phagocytosis of B16-WT, B16-SKO, B16-CCL5 KO cells irradiated by X-rays at 50 Gy. (D-F) 5×10⁵ of B16-OVA-WT, B16-OVA-SKO or B16-OVA-CCL5 cells were inoculated into the right flank of WT CL57/B6 mice (n=6/group). 7 days after inoculation, tumors were locally exposed to X-rays at 8 Gy, three times consecutively. (D) Tumor volume was measured every other day for 2 weeks. (E) Tumor weights were measured at the end of the experiment (15 days after inoculation). (F) IFNγ ELISPOT assays against OVA antigen using CD8+ T cells isolated from the same mice depicted as in (D). (G) B16-OVA-WT. B16-OVA-SKO or B16-OVA-CKO cells were reconstituted with pCMV-CCL5-Flag. CCL5 expression is shown by immunoblotting using anti-Flag. qPCR analysis of IFNβ1 and Cxcl10 in WTØ following the phagocytosis of irradiated B16-OVA-WT, B16-OVA-SKO or B16-OVA-CKO with or without overexpression of CCL5 after X-rays at 50 Gy. (H-J) 5×10⁵ of B16-OVA-SKO or B16-OVA-SKO+CCL5 cells were inoculated into the right flank of WT CL57/B6 mice (n=6/group). 7 days after inoculation, tumors were locally exposed to X-rays at 8 Gy three times consecutively. (H) Tumor volume was measured every other day for 2 weeks. (I) Tumor weights were measured at the end of the experiment (15 days after inoculation). (J) IFNγ ELISPOT assay against OVA antigen using CD8+ T cells isolated from the same mice as depicted in (H). (K) Immunofluorescent analysis using anti-dsDNA antibody in irradiated WT. SKO or CCL5 KO MEFs at 50 Gy after 3 days. Original magnification ×200. (L) Number of cytoplasmic DNA foci were counted in 20 different cells from each group and the average calculated. (M) Flow cytometry analysis for macrophage engulfment efficiency following the phagocytosis of CFSE-labeled B16-WT, B16-SKO or B16-CCL5 KO cells for 6 h, by flow cytometry. (N) cGAMP ELISA assays in WT DCs, SKO DC, and CKO DC following the phagocytosis of MEF-WT, MEF-SKO, and MEF-CCL5 KO cells irradiated with X-rays at 50 Gy. CD11c+ BMDC cells were isolated by CD11c microbeads (Miltenyi Biotec) 6 h after phagocytosis of the irradiated cells. (O) ELISA analysis of CCL5 in WT, SKO or CCL5 KO MEFs treated with 300 ng/mL IFNα for 6 h. (P, Q) 5×10⁵ B16-SKO cells were inoculated into the flanks of C57/BL6 mice (n=6 per group) and then incubated for 7 days. IFNα (0.5 μg/mouse) was introduced by i.p. injection 3 times every 2 days. PBS was used as vehicle control. After the first injection of IFNα, tumors were irradiated using 8 Gy of X-ray every other day for a total of three times. (P) Tumor volumes were evaluated every other day for 14 days. (Q) IFNγ ELISPOT assay against OVA antigen using CD8+ T cells isolated from the same mice depicted in (P). (R) qPCR analysis of Cxcl10 in WT CD11c+ BMDCs following the phagocytosis of irradiated WT, SKO or CCL5 MEFs treated with 300 ng/mL IFNα. Data is representative of at least three independent experiments. Error bars indicate mean±SD. *p<0.05; Student's t-test, according to various embodiments of the present invention;

FIG. 4 shows Characterization of STING-inducible endosomal vesicles (SIEVE's). SIEVES were isolated from irradiated (X-ray at 50 Gy for 3 days) WT, SKO, or CCL5 MEFs as well as B16-WT, B16-SKO or B16-CCL5KO cells by centrifugation. (A) Immunoblot analysis of centrifuged fractions using antibodies to STING, CD81, LC3, and Lamin A/C. (B) Size distribution evaluation of endosomal vesicles isolated from B16-WT. B16-SKO, and B16-CCL5 KO cells following irradiation with X-rays at 50 Gy. (C) Evaluation of number of endosomal vesicles in (B). (D) Immunoblotting analysis of endosomal/exosomal markers in endosomal vesicles in (B). (E) cGAMP ELISA analysis of WT DC. SKO DC, and CKO BMDC's treated with 30 μg of SIEVE's isolated from MEF WT, MEF SKO, MEF CKO or MEF CCL5 KO. (F) qPCR analysis of Cxcl10 in WT DC treated with SIEVE's from (E). (G-I) C57/BL6 mice were inoculated with 5×10⁵ of B16-OVA-WT cells on the flanks (n=6/group). 7 days after inoculation, 50 μg of SIEVE's isolated from irradiated B16-WT or B16-SKO cells were intratumorally inoculated three times consecutively. (G) Tumor volumes were measured every other day for 2 weeks. (H) IFNγ ELISPOT assay against OVA antigen using CD8+ T cells isolated from the same mice as depicted in (H). (I) ELISA concentration of CCL5 in SIEVE's isolated from B16 WT. SKO or CCL5 KO cells exposed to X-rays at 50 Gy. (J) Picogreen staining analysis of different concentration of CC15 and control S100A8 incubated with ISD (10 μg/mL). Data was showed as percentage of fluorescence with respect to the fluorescence of ISD alone (100%). (K) Efficiency of DNaseII digestion in the presence of CCL5 at the designated concentration. (L) Picogreen analysis in endosomal vesicles isolated from B16 WT, or SKO CCL5 KO cells. (M) qPCR analysis of IFNβ1 and Cxcl10 in CD11+ BMDC's following the phagocytosis of MEFs treated with the autophagy inhibitors, Bafilomycin A1 (BafA1, 50 nM) or 3-Methyladenine (3-MA, 2.5 mM). (N) qPCR of Cxcl10 in WT macrophages following the phagocytosis of irradiated MEFs exposed to X-rays at 50 Gy, pre-treated with RNAi to autophagy related genes. MEF WT cells were transfected with 100 nM siRNAs 1 day prior to irradiation. Data is representative of at least three independent experiments. Error bars indicate mean±SD. *p<0.05; Student's t-test, according to various embodiments of the present invention;

FIG. 5 shows (A) Immunoblot analysis of STING and cGAS in MEF-WT, MEF-WT-SKO, and MEF-WT-CKO cells treated with 50 Gy X-ray for 3 days. (B) Immunofluorescent analysis using anti-dsDNA in the irradiated MEF-WT. MEF-WT-SKO, or MEF-WT-CKO cells as depicted in (A). Original magnification X200. (C) Number of cytoplasmic DNA spots in (B) were quantitated from 20 different cells in each group and the average was calculated. (D) Quantitation of dsDNA by Picogreen staining in cytoplasmic extracts from MEF-WT, MEF-WT-SKO, or MEF-WT-CKO cells as in (B). (E) qPCR analysis of Cxcl10 in WT, SKO or CKO MEFs irradiated by X-rays at 50 Gy and followed for 3 days. (F) cGAMP ELISA assay of MEF-WT, MEF-SKO or MEF-CKO cells at 3 days following radiation at 50 Gy. (G) qPCR analysis of IFNβ1 and Cxcl10 in WTØ APC's following the phagocytosis of MEF-WT, MEF-SKO or MEF-CKO cells for 6 h, according to various embodiments of the present invention;

FIG. 6 shows tumor weights from the mice shown in FIG. 1L. (B) WT and SKO B16 cells (5×10⁵ cells/mouse) were inoculated into flank of WT (n=6/group) or SKO C57/BL6 mice (n=7/group) followed by treatment of the tumors with X-ray (8 Gy) on day 7, 8, and 9. Tumor volumes were monitored every other day for 14 days, according to various embodiments of the present invention;

FIG. 7 shows immunohistochemistry analysis using shown antibodies, of tumor tissue from WT mice inoculated with C1498 cells as shown in FIG. 1L according to an embodiment of the present invention;

FIG. 8 shows EL4 cells reconstituted with pcDNA-cGAS (EL4-cGAS) or empty vector (EL4-Vec). The cells were treated by X-ray radiation at 50 Gy. (A) Immunoblot analysis of TBK1, IRF3, cGAS or STING in irradiated EL4-OVA and EL4-OVA-cGAS. (B) qPCR analysis of Ifnβ1, Cxcl10, or CCL5 in the same cells depicted in (A). (C) ELISA analysis of cGAMP in EL4 cells reconstituted with cGAS at the designated time points. (D) Schematic representation of the phagocytosis of irradiated EL4-OVA cells. EL4-OVA or EL4-OVA-cGAS were irradiated by X-rays (50 Gy) and fed to bone marrow derived macrophages (WTØ, SKOØ, CKOØ). (E) qPCR analysis of Ifnβ1 or Cxcl10 in WT. SKO, or CKO macrophages following the phagocytosis of irradiated EL4 cells. (F-G) EL4 cells containing or lacking cGAS were inoculated into C57/BL6 mice (n=7/group). EL4 or EL4-cGAS tumors established in WT mice were treated locally with X-ray (8 Gy) every day for total of three days. (F) Tumor growth was monitored every other day for 18 days. (G) IFNγ ELISPOT assay against OVA antigen using CD8+ T cells isolated from the same mice as in (F), according to an embodiment of the present invention;

FIG. 9 shows (A and B) WT A375 or reconstituted cGAS expressing A375 cells (A375-cGAS) were treated with 10 Gy, X-ray for indicated times. (A) Immunoblot analysis of STING, cGAS, TBK1 or IRF3 in irradiated A375 and A375-cGAS cells with X-ray at 10 Gy. followed by measuring STING signal activity by immunoblot assay. (B) qPCR analysis of Ifnβ1 and Cxcl10 in the same cells as depicted in (A). (C-D) A375 cells were treated with 5-Azadc at 1 uM for 3 days and then irradiated by X-ray (10 Gy) for indicated times. (C) Immunoblot analysis and (D) qPCR analysis was performed. (E) Schematic of phagocytosis of WT or SKO A375 cells with 5-Azade in WT BMDM (WTØ). WT and SKO A375 cells pre-treated with 1 μM 5-Azadc for 3 days, were irradiated with X-ray (50 Gy) for 3 days and then fed to WT BMDM for 6 h. (F) qPCR analysis of Ifnβ1 or Cxcl10 in WT BMDM following the phagocytosis of irradiated A375 and A375-SKO cells, according to an embodiment of the present invention;

FIG. 10 shows (A) qPCR analysis of Ifnβ1 or Cxcl10 in WT macrophages (WTØ) following the phagocytosis of WT MEFs transfected with different siRNAs at 100 nM for 1 day followed by X-ray irradiation (50 Gy). 3 days after irradiation, 2×10⁶ cells were phagocyted by 2×10⁵ macrophages for 6 h. After washing, mRNA was extracted with Trizol and the indicated mRNA levels were analyzed by qRT-PCR (B) WT, CCL5 KO, SAA3 KO and S100A8 KO B16 cells were irradiated by X-ray (50 Gy) and incubated for 3 days and then phagocyted by macrophages (2×10⁵ cells) for 6 h. After washing, Ifnβ1 or Cxcl10 mRNA level from the macrophages was measured by qRT-PCR, according to an embodiment of the present invention.

FIG. 11 shows (A) WT macrophages (WTØ) were pre-treated with recombinant CCL5 protein at 100 ng/mL for 2 h. qPCR analysis of Cxcl10 in WTØ pretreated with or without CCL5 following the phagocytosis of B16-WT and B16-SKO irradiated by X-rays at 50 Gy for 3 days. (B) B16-WT or B16-SKO cells were irradiated using X-rays at 50 Gy for 3 days. 50 μL of the conditioned media from the cells was added to WTØ followed by adding the irradiated B16-WT and B16-SKO cells with the treated APC's for phagocytosis analysis. qPCR analysis was performed for Cxcl10 expression. (C) qPCR analysis of Cxcl10 in WTØ following the phagocytosis of irradiated MEF-WT cells in the presence of CCL5 antibody or IgG control (1 μg) at 100 ng/mL for 2 h prior to the phagocytosis. (D) MEF-WT cells were treated with a cocktail of 100 nM CCR inhibitors (Bx471, SB297006, AZD2098 and Maraviroc). The MEF-WT cells treated with the inhibitors (CCRi) were fed to WTØ for 6 h and Cxcl10 expression examined. (E) MEF WT, SKO or CCL5 KO were transfected with 3 μg/mL ISD for 6 h followed by measuring Ifnβ1, Cxcl10 or Ccl5 expression by qPCR analysis. (F) MEF WT, SKO or CCL5 KO were transfected with 3 μg/mL ISD for 6 h and then examined for STING activity by immunoblotting analysis. (G) WT, SKO or CCL5 B16 cells were placed into coverslips and incubated for 24 h followed by transfection with 3 μg/mL ISD for 3 h. Immunofluorescence to detect STING translocation was monitored by confocal microscope, according to an embodiment of the present invention;

FIG. 12 shows (A) ISD (10 μg/mL) was premixed with the indicated concentration of CCL5 followed by visualization of CCL5-ISD by agarose gel electrophoresis. (B) B16-WT. SKO and CCL5 KO cells were transfected with ISD (3 μg/mL) for 6 h and then the lysed cells were incubated with CCL5 antibody conjugated plate for 2 h. DNA was purified with phenol-chloroform and CCL5 bound ISD was analyzed by qPCR analysis. (C) DNA in SIEVE's isolated from MEF-WT, SKO and CCL5 KO with or without X-ray at 50 Gy were measured by Nanodrop, Picogreen staining and visualized by agarose gel electrophoresis and dot blot analysis, according to an embodiment of the present invention; and

FIG. 13 shows (A) Proteomics profile of STING-inducible proteins isolated from SIEVE's purified from irradiated WT or SKO MEFs. Proteomics analysis of proteins were conducted using an Alpha Nano Tech, LLC (Morrisveill, NC, USA) LC-MS/MS with a Thermo Easy nLC 1200-QEactive HF. Raw data were analyzed using proteome Discoverer 2.5—searched against the Uniprot mouse database (containing ˜17,000 protein sequences) and a common contaminants database (containing ˜245 sequences). Further analysis and statistics (Log2 transformation, imputation, t-test) were conducted by Perseus. WT MEFs were pre-transfected with 100 nM of the indicated siRNA's for 1 day before irradiating them using X-rays (50 Gy). MEFs were incubated (phagocytosed) with WT macrophages for 6 h and Cxcl10 expression measured by qPCR analysis. (B) BMDCs were incubated with irradiated WT. SKO, Trex1 knockout (TKO) or Sting/Trex1 double knockout (STKO) (50 Gy, X-ray for 3 days). qPCR analysis of Ifit1 and Cxcl10 from purified CD11c+ BMDCs (DCs) was carried out, according to an embodiment of the present invention.

### DETAILED DESCRIPTION OF THE INVENTION

### Definitions

Listed below are definitions of various terms used in this application. These definitions apply to the terms as they are used throughout this specification and claims, unless otherwise limited in specific instances, either individually or as part of a larger group.

The transitional term ‘comprising’ is synonymous with ‘including’, ‘containing,’ or ‘characterized by’ is inclusive or open-ended and does not exclude additional, unrecited elements or method steps. The transitional phrase ‘consisting of’ excludes any element, step, or ingredient not specified in the claim, but does not exclude additional components or steps that are unrelated to the invention such as impurities ordinarily associated with a composition. The transitional phrase ‘consisting essentially of’ limits the scope of a claim to the specified materials or steps and those that do not materially affect the basic and novel characteristic(s) of the claimed invention.

The term ‘subject’ as used herein refers to a mammal. A subject therefore refers to, for example, dogs, cats, horses, cows, sheep, pigs, guinea pigs, rats, mice, monkeys, apes and the like. Preferably the subject is a human. When the subject is a human, the subject may be referred to herein as a patient.

The words ‘treat’, ‘treating’ and ‘treatment’ refer to a method of alleviating or abating a disease and/or its attendant symptoms.

The words ‘preventing’ and ‘prevent’ describe reducing or eliminating the onset of the symptoms or complications of the disease, condition or disorder.

The terms ‘disease(s)’, ‘disorder(s)’, and ‘condition(s)’ are used interchangeably, unless the context clearly dictates otherwise.

The term ‘therapeutically effective amount’ of a compound or pharmaceutical composition of the application, as used herein, means a sufficient amount of the compound or pharmaceutical composition so as to decrease the symptoms of a disorder in a subject. As is well understood in the medical arts a therapeutically effective amount of a compound or pharmaceutical composition of this application will be at a reasonable benefit/risk ratio applicable to any medical treatment. It will be understood, however, that the total daily usage of the compounds and compositions of the present application will be decided by the attending physician within the scope of sound medical judgment. The specific modulatory (e.g., inhibitory or stimulatory) dose for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

As used herein, the phrase ‘pharmaceutically acceptable’ refers to those compounds, materials, compositions, carriers, and/or dosage forms which are, within the scope of sound medical judgment, suitable for use in contact with the tissues of human beings and animals without excessive toxicity. irritation, allergic response, or other problem or complication, commensurate with a reasonable benefit/risk ratio.

As used herein, the term ‘pharmaceutically acceptable salt’ refers to those salts of the compounds formed by the process of the present application which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example. S. M. Berge, et al. describes pharmaceutically acceptable salts in detail in J. Pharmaceutical Sciences, 66:1-19 (1977), which is herein expressly incorporated by reference in its entirety and for all purposes. The salts can be prepared in situ during the final isolation and purification of the compounds of the application, or separately by reacting the free base or acid function with a suitable acid or base.

Examples of pharmaceutically acceptable salts include, but are not limited to, nontoxic acid addition salts: salts formed with inorganic acids such as hydrochloric acid, hydrobromic acid, phosphoric acid, sulfuric acid and perchloric acid, or with organic acids such as acetic acid, maleic acid, tartaric acid, citric acid, succinic acid or malonic acid. Other pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate propionate, stearate, succinate, sulfate, tartrate, thiocyanate, 7-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counter ions such as halide, hydroxide, carboxylate, sulfate, phosphate, nitrate, alkyl having from 1 to 6 carbon atoms, sulfonate and aryl sulfonate.

As used herein, the term ‘pharmaceutically acceptable ester’ refers to esters of the compounds formed by the process of the present application which hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms.

Examples of particular esters include, but are not limited to, formates, acetates, propionates, butyrates, acrylates and ethylsuccinates.

The term ‘pharmaceutically acceptable prodrugs’ as used herein, refers to those prodrugs of the compounds formed by the process of the present application which are, within the scope of sound medical judgment, suitable for use in contact with the tissues of humans and lower animals with undue toxicity, irritation, allergic response, and the like, commensurate with a reasonable benefit/risk ratio, and effective for their intended use, as well as the zwitterionic forms, where possible, of the compounds of the present application.

Prodrug', as used herein, means a compound which is convertible in vivo by metabolic means (e.g., by hydrolysis) to afford any compound delineated by the formulae of the instant application. Various forms of prodrugs are known in the art, for example, as discussed in Bundgaard, (ed.), Design of Prodrugs, Elsevier (1985); Widder, et al. (ed.), Methods in Enzymology, vol. 4, Academic Press (1985); Krogsgaard-Larsen, et al., (ed). “Design and Application of Prodrugs, Textbook of Drug Design and Development, Chapter 5, 113-191 (1991); Bundgaard, et al., Journal of Drug Deliver Reviews, 8:1-38 (1992); Bundgaard, J. of Pharmaceutical Sciences, 77:285 et seq. (1988); Higuchi and Stella (eds.) Prodrugs as Novel Drug Delivery Systems, American Chemical Society (1975); and Bernard Testa & Joachim Mayer, “Hydrolysis In Drug And Prodrug Metabolism: Chemistry, Biochemistry And Enzymology.” John Wiley and Sons, Ltd. (2002), which are all herein expressly incorporated by reference in their entireties and for all purposes.

Pharmaceutically acceptable excipient' means an excipient that is useful in preparing a pharmaceutical composition that is generally safe, non-toxic and neither biologically nor otherwise undesirable, and includes an excipient that is acceptable for veterinary use as well as human pharmaceutical use. A pharmaceutically acceptable excipient as used in the specification and claims includes both one and more than one such excipient.

This application also encompasses pharmaceutical compositions containing, and methods of treating disorders through administering, pharmaceutically acceptable prodrugs of compounds of the application. For example, compounds of the application having free amino, amido, hydroxy or carboxylic groups can be converted into prodrugs. Prodrugs include compounds wherein an amino acid residue, or a polypeptide chain of two or more (e.g., two, three or four) amino acid residues is covalently joined through an amide or ester bond to a free amino, hydroxy or carboxylic acid group of compounds of the application. The amino acid residues include but are not limited to the 20 naturally occurring amino acids commonly designated by three letter symbols and also includes 4-hydroxyproline, hydroxylysine, demosine, isodemosine, 3-methylhistidine, norvalin, beta-alanine, gamma-aminobutyric acid, citrulline, homocysteine, homoserine, ornithine and methionine sulfone. Additional types of prodrugs are also encompassed. For instance, free carboxyl groups can be derivatized as amides or alkyl esters. Free hydroxy groups may be derivatized using groups including but not limited to hemisuccinates, phosphate esters, dimethylaminoacetates, and phosphoryloxymethyloxy carbonyls, as outlined in Advanced Drug Delivery Reviews, 1996, 19, 1-15, which is herein expressly incorporated by reference in its entirety and for all purposes. Carbamate prodrugs of hydroxy and amino groups are also included, as are carbonate prodrugs, sulfonate esters and sulfate esters of hydroxy groups. Derivatization of hydroxy groups as (acyloxy)methyl and (acyloxy)ethyl ethers wherein the acyl group may be an alkyl ester, optionally substituted with groups including but not limited to ether, amine and carboxylic acid functionalities, or where the acyl group is an amino acid ester as described above, are also encompassed. Prodrugs of this type are described in J. Med. Chem. 1996, 39, 10, which is herein expressly incorporated by reference in its entirety and for all purposes. Free amines can also be derivatized as amides sulfonamides or phosphonamides. All of these prodrug moieties may incorporate groups including but not limited to ether, amine and carboxylic acid functionalities.

Combinations of substituents and variables envisioned by this application are only those that result in the formation of stable compounds. The term ‘stable’, as used herein, refers to compounds which possess stability sufficient to allow manufacture and which maintains the integrity of the compound for a sufficient period of time to be useful for the purposes detailed herein (e.g., therapeutic or prophylactic administration to a subject).

Additionally, the compounds of the present application, for example, the salts of the compounds, can exist in either hydrated or unhydrated (the anhydrous) form or as solvates with other solvent molecules. Non-limiting examples of hydrates include monohydrates, dihydrates, etc. Non-limiting examples of solvates include ethanol solvates, acetone solvates, etc.

‘Solvate’ means solvent addition forms that contain either stoichiometric or non-stoichiometric amounts of solvent. Some compounds have a tendency to trap a fixed molar ratio of solvent molecules in the crystalline solid state, thus forming a solvate. If the solvent is water, the solvate formed is a hydrate; and if the solvent is alcohol, the solvate formed is an alcoholate. Hydrates are formed by the combination of one or more molecules of water with one molecule of the substance in which the water retains its molecular state as H₂O.

In the present specification, the structural formula of the compound represents a certain isomer for convenience in some cases, but the present application includes all isomers, such as geometrical isomers, optical isomers based on an asymmetrical carbon, stereoisomers, tautomers, and the like.

All documents mentioned herein are incorporated herein by reference. All publications and patent documents cited in this application are incorporated by reference for all purposes to the same extent as if each individual publication or patent document were so individually denoted. By their citation of various references in this document, Applicants do not admit any particular reference is ‘prior art’ to their invention. Embodiments of inventive compositions and methods are illustrated in the following examples.

STING is a cellular innate immune receptor essential for controlling the transcription of numerous host defense genes, including type I IFN and pro-inflammatory cytokines following the recognition of CDN's or aberrant DNA species in the cytosol of the cell. The source of DNA can comprise the genome of invading pathogens such as herpes simplex 1 virus (HSV1) or CDNs which are known to be secreted by bacteria such as Listeria monocytogenes. That is, STING can directly sense CDNs including c-di-GMP or c-di-AMP secreted by invading intracellular bacteria, cyclic-GMP-AMP (cGAMP) generated by the cellular synthase, Cyclic GMP-AMP synthase (cGAS) following association with cytosolic dsDNA species such as microbial DNA, or self-DNA. Generally, the cytosol of the cell is free of DNA, since it can aggravate STING-dependent cytokine production, an event that can lead to lethal auto-inflammatory disease. For example, self-DNA leaked from the nucleus of cells, following cell division or following DNA damage, is prevented from activating STING signaling by the exonuclease DNase III (Trex1). Consequently, defects in Trex1 function can lead to severe auto-inflammatory diseases due to undigested self-DNA triggering STING activity. In addition, following the engulfment of apoptotic cells, phagocyte-dependent DNase II plays a critical role in digesting the DNA from the dead cell, to prevent it from activating STING-signaling extrinsically within the phagocyte.

As used herein the phrase ‘STING intracellular pathways’ include IRF-3 and NF-KB pathways.

Self-DNA leaked from the nucleus of the host cell, following cell division or even as a consequence of DNA damage can activate STING. Such self-DNA may be responsible for causing a variety of auto-inflammatory disease such as Systemic Lupus Erythamatosis (SLE) or Aicardi-Goutieres Syndrome (AGS) and may even be associated with inflammation-associated cancer. Recent insight into the regulation of STING signaling has generated much needed information relating to the causes of inflammatory disease, providing new opportunities to develop novel anti-inflammatory compounds that target this pathway.

The ability of dying cells to activate Antigen Presenting Cells (APCs) is carefully controlled to avoid unwanted inflammatory responses. For example, following phagocytosis, regular dying cells do not trigger inflammatory responses which can be required for efficient cytotoxic T lymphocyte (CTL) priming of the immune system.

Tumor cells presumably mimic these processes to avoid activating APCs. However, dying tumor cells contain exogenous innate immune agonists such as cytosolic DNA. The cytosolic DNA can potently activate APCs in trans through extrinsic innate immune, STING-dependent signaling, to generate potent Cytotoxic T Lymphocyte (CTL) activity. In the absence of STING agonists, dying cells are ineffectual in the stimulation of APCs in trans. Indeed, cytosolic STING activators, including cytosolic DNA and cyclic dinucleotides (CDNs), constitute cellular danger associated molecular patterns (DAMPs) usually only generated by viral infection or following DNA-damage events, that can render tumor cells highly immunogenic (i.e., STING activators make a ‘cold’ tumor ‘hot’).

Tumor cells often involve the efficient eradication of apoptotic cells designed to avoid invoking an inflammatory event. Dying cells are generally poor activators of phagocytes and are immunologically indolent due to the genomic DNA being degraded by host DNases to prevent the intrinsic and extrinsic activation of STING. Tumor cells mimic this efficient process and avoid activating anti-tumor CTL activity. However, cancer cells containing cytosolic dsDNA species, that escape degradation, can potently stimulate APCs, via extrinsic STING-signaling, to promote the cross-presentation of tumor antigen.

STING is activated by cyclic dinucleotides (CDNs) such as cyclic di-GMP and cyclic-di-AMP secreted by intracellular bacteria following infection. Alternatively, STING can be activated by cyclic GMP-AMP (cGAMP) generated by a cellular cGAMP synthase cGAS after association with aberrant cytosolic dsDNA species, which can include microbial DNA or self-DNA leaked from the nucleus. Notably, STING signaling has been shown to be important for facilitating anti-tumor T cell activity. Cytosolic dsDNA species present within a dying tumor cell can activate extrinsic STING signaling in phagocytes likely following association with cGAS which can generate CDNs.

The phrase ‘STing Dependent Adju Vants’ or ‘STAVs’ refers to dsDNA oligonucleotides of approximately 70 bp which are innate immune activators of STING. In this instance approximately refers to plus or minus twenty percent (20%). The STAV compositions of the present invention comprise at least one modification which confers increased or enhanced stability to the STA Vs, including, for example, improved resistance to nuclease digestion in vivo. In an embodiment, the STAV compositions of the present invention have undergone a chemical or biological modification to render them more stable. Exemplary modifications to the STAVs include the modification of a base, for example, the chemical modification of a base.

The term ‘functional’ as used herein means that the STAV has biological activity to activate STING. The STA compositions of the invention are useful for the treatment of cancer, inflammation and other disorders. The term ‘therapeutic levels’ refers to levels of STAVs above normal physiological levels, or the levels in the subject prior to administration of the STAV composition. As provided herein. the compositions include a transfer vehicle. As used herein, the term ‘transfer vehicle’ includes any of the standard pharmaceutical carriers, diluents, excipients and the like which are generally intended for use in connection with the administration of biologically active agents, including nucleic acids. The compositions and in particular the transfer vehicles described herein are capable of delivering STAVs to the target cell. In embodiments, the transfer vehicle is a lipid nanoparticle.

The term ‘polymer-conjugated lipid’ means a polymer (for example, polyethylene glycol (PEG), polypropylene glycol, polyvinylpyrrolidone, poly(N-(2-hydroxypropyl) methacrylamide) s and PEGylated liposomes with different functional groups, including methoxy (OCH₃), amino (NH₂), carboxyl (COOH), and hydroxyl (OH) moieties) conjugated with a lipid. For example, PEG can be conjugated with myristoyl diglyceride to generate DMG-PEG 2000. Alternatively, PEG can be conjugated with DSPE a water-soluble derivative of phosphatidylethanolamine with (18:0) stearic acid acyl chains to generate DSPE PEG 2000. PEG conjugated lipids can incorporate various functionalized PEG terminal groups including amine, carboxylic acid, azide, aldehyde, thiol, and hydroxyl moieties. PEG conjugated lipids improve circulation times, drug stability, suitability of different routes of administration, and help achieve targeted drug delivery. In an alternative embodiment of the present invention, a branched polymer (e.g., poly(oligo(ethylene glycol) methyl ether methacrylate, i.e., poly(tri(ethylene glycol) methyl ether methacrylate, poly(tetra(ethylene glycol) methyl ether methacrylate. poly(penta (ethylene glycol)methyl ether methacrylate, poly(hexa(ethylene glycol) methyl ether methacrylate, poly(hepta(ethylene glycol) methyl ether methacrylate, poly(octa(ethylene glycol) methyl ether methacrylate, poly(noan(ethylene glycol) methyl ether methacrylate) can be conjugated with lipids.

In addition to PEG conjugated lipids, a ‘sterol’ or an unsaturated steroid alcohol, can be used to enhance the stability of the LNP. Sterols can include natural sterols and sterols with unnatural ring junctions. Sterols can be used to assist the efficiency of introducing the STAV into the cells. Changing the nature of the sterol component can also be used to alter the efficiency of introducing the STAV into cells. Natural sterols include cholesterol, cholesterol sulfate, desmosterol, stigmasterol, lanosterol, 7-dehydrocholesterol, dihydrolanosterol, zymosterol, lathosterol, 14-demethyl-lanosterol, 8(9)-dehydrocholesterol, 8(14)-dehydrocholesterol, FF-MAS, diosgenin, dehydroepiandrosterone (DHEA) sulfate, DHEA, sitosterol, lanosterol-95, zymostenol, sitostanol, campestanol, campesterol, 7-dehydrodesmosterol, pregnenolone, dihydro T-MAS, delta 5-avenasterol, brassicasterol, dihydro FF-MAS, 24-methylene cholesterol, 3β-hydroxy-7-oxo-5-cholestenoic acid, 7α-hydroxy-3-oxo-4-cholestenoic acid, 3β,7α-dihydroxy-5-cholestenoic acid, 3β,7β-dihydroxy-5-cholestenoic acid, 3β-hydroxy-5-cholestenoic acid, 3-oxo-4-cholestenoic acid, 3β,7α,24S-trihydroxy-5-cholestenoic acid, 3β,24S-dihydroxy-5-cholestenoic acid, 3β,7α,25-trihydroxy-5-cholestenoic acid, and 3β,25-OH-7-oxo-5-cholestenoic acid.

A ‘phospholipid’ means a molecule with a hydrophilic head group and an aliphatic chain linked to an alcohol moiety. The nature of the head group, the aliphatic chain and the alcohol can be used to generate a wide variety of phospholipids. The aliphatic chain includes saturated acyl chains, saturated alkyl chains, unsaturated acyl chains, unsaturated alkyl chains, saturated acyl chains with ether bonds, saturated alkyl chains with ester bonds, unsaturated acyl chains with ether bonds and unsaturated alkyl chains with ester bonds. Glycerophospholipids and sphingomyelins are phospholipids which differ based on the alcohol moieties. Phospholipids include phosphatidylcholine, phosphatidylethanolamine, phosphatidylserine, phosphatidic acid, phosphatidylinositol, phosphatidylglycerol, cardiolipin, dipalmitoyl, dimyristoyl, distearoyl phosphatidylcholine, dioleoyl, distearoyl PC, and L-α-phosphatidylcholine. In an embodiment of the present invention, the alcohol in the phospholipid can be a C3 alcohol. In an alternative embodiment of the present invention, the phospholipid can include a C4-C8alcohol.

An ‘ionizing lipid’ is a class of lipid molecules which remain neutral at physiological pH, but are protonated under acidic conditions. Ionizing lipids promote endosome escape and reduce toxicity of the LNP. Ionizable lipids include 7-[(2-Hydroxyethyl)[8-(nonyloxy)-8-oxooctyl]amino]heptyl 2-octyldecanoate, DODMA (MBN 305A), DLin-KC2-DMA, (6Z,9Z,28Z,31Z)-Heptatriaconta-6,9,28,31-tetraen-19-yl 4-(dimethylamino)butanoate (D-Lin-MC3-DMA, or MC3), Heptadecan-9-yl8-{(2-hydroxyethyl)[6-oxo-6-(undecyloxy)hexyl]amino}octanoate (SM-102), and [(4-Hydroxybutyl)azanediyl]di(hexane-6,1-diyl) bis(2-hexyldecanoate) (ALC-0315).

The term ‘LNP’ means a lipid nanoparticle. A LNP represents a particle made from lipids (e.g., cationic lipids, non-cationic lipids, conjugated lipids and/or a sterol that prevents aggregation of the nanoparticle), and a STAV, where the STAV is encapsulated within the lipid (e.g., NanoSTAVs).

In an embodiment of the present invention, LNP formulations can have four major components, other than the nucleic acid. In an embodiment of the present invention, a LNP comprises a phospholipid, a sterol, an ionizable lipid, and a polymer-conjugated lipid. In an embodiment of the present invention, the phospho lipid can be distearoylphosphatidylcholine, the sterol can be cholesterol, the ionizable lipid can be MC3, and PEG-conjugated lipid can be DMG-PEG 2000 to generate STAV1 a double-stranded polyA:T76ES, oligonucleotides (SEQ ID NO:24)+(SEQ ID NO:25); STAV2 a double-stranded polyAC:TG76ES, oligonucleotides (SEQ ID NO:26)+(SEQ ID NO:27); and STAV3 a double-stranded polyAT:TA76ES, oligonucleotides (SEQ ID NO:37)+(SEQ ID NO:38), where the diameter of the spherical lipo-nanoparticles can be approximately 88 nm, where approximately means +−10 nm. In an embodiment of the present invention, the cholesterol can be between 35-45% of the LNP composition. In an embodiment of the present invention, the LNP comprises a distearoylphosphatidylcholine (DSPC), cholesterol, an MC3-like lipid and a PEG-conjugated lipid. The phospholipid and cholesterol promote stability and structural integrity of the LNP. The ionizable lipid promotes electrostatic interaction with the negatively charged nucleic acids and helps intracellular delivery. The polymer-conjugated lipid improves solubility of the LNP in serum, and circulation by preventing the particles from aggregating, while retaining good biocompatibility and having good tolerance characteristics. In an embodiment of the present invention, the Nano-STAVs were composed of 76 bp of dsDNA modified with phosphorothioate to block exonuclease activity, encapsulated at a nitrogen to phosphate mole ratio of approximately 6 (where approximately means plus or minus one). In an embodiment of the present invention, the Nano-STAV particles can be approximately 88 nm in size, where approximately means plus or minus ten (10) per cent. In an embodiment of the present invention, the STAVS are approximately 50% encapsulated in the Nano-STAVS. In this range approximately means plus or minus twenty (20) per cent. In an alternative embodiment of the present invention, the STAVS are approximately 75% encapsulated in the Nano-STAVS. In this range approximately means plus or minus ten (10) per cent. In another embodiment of the present invention, the STAVS are at least approximately 90% encapsulated in the Nano-STAVS. In this range approximately means plus or minus five (5) per cent. In another alternative embodiment of the present invention, the STAVS are approximately 98% encapsulated in the Nano-STAVS. In this range approximately means plus or minus one (1) per cent. In an embodiment of the present invention, the STAVS are approximately 98% encapsulated in the Nano-STAVS, at a concentration of dsDNA in the LNP in PBS of 0.2 mg/mL. LNP are extremely useful for systemic applications, as they can exhibit extended circulation lifetimes following intravenous (i.v.) injection, they can accumulate at distal sites (e.g., sites physically separated from the administration site), and they can deliver the STAVs at sites distal to the site of administration.

The methods of the invention provide for optional co-delivery of one or more unique STAVs to target cells, for example, by combining two unique STAVs into a single transfer vehicle. In an embodiment of the present invention, a therapeutic first STAV, and a therapeutic second STAV, can be formulated in a single transfer vehicle and administered. The present invention also contemplates co-delivery and/or co-administration of a therapeutic first STAV and a second STAV to facilitate and/or enhance the function or delivery of one or both the therapeutic first STAV and the therapeutic second STAV.

Pharmaceutical compositions including a compound of the present application in free form or in a pharmaceutically acceptable salt form in association with at least one pharmaceutically acceptable carrier or diluent may be manufactured in a conventional manner by mixing, granulating or coating methods. For example, oral compositions can be tablets or gelatin capsules comprising the active ingredient together with a) diluents, e.g., lactose, dextrose, sucrose, mannitol, sorbitol, cellulose and/or glycine; b) lubricants, e.g., silica, talcum, stearic acid, its magnesium or calcium salt and/or polyethyleneglycol; for tablets also c) binders, e.g., magnesium aluminum silicate, starch paste, gelatin, tragacanth, methylcellulose, sodium carboxymethylcellulose and or polyvinylpyrrolidone; if desired d) disintegrants, e.g., starches, agar, alginic acid or its sodium salt, or effervescent mixtures; and/or e) absorbents, colorants, flavors and sweeteners. Injectable compositions can be aqueous isotonic solutions or suspensions, and suppositories can be prepared from fatty emulsions or suspensions. The compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Suitable formulations for transdermal applications include an effective amount of a compound of the present application with a carrier. A carrier may include absorbable pharmacologically acceptable solvents to assist passage through the skin of the host. For example, transdermal devices may be in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin. Matrix transdermal formulations may also be used. Suitable formulations for topical application, e.g., to the skin and eyes, are preferably aqueous solutions, ointments, creams or gels well-known in the art. Such may contain solubilizers, stabilizers, tonicity enhancing agents, buffers and preservatives.

The pharmaceutical compositions of the present application comprise a therapeutically effective amount of a compound of the present application formulated together with one or more pharmaceutically acceptable carriers. As used herein, the term ‘pharmaceutically acceptable carrier’ means a non-toxic, inert solid, semi-solid or liquid filler, diluent, encapsulating material or formulation auxiliary of any type. Some examples of materials which may serve as pharmaceutically acceptable carriers include, but are not limited to, ion exchangers, alumina, aluminum stearate, lecithin, serum proteins, such as human serum albumin, buffer substances such as phosphates, glycine, sorbic acid, or potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, polyacrylates, waxes, polyethylenepolyoxy propylene-block polymers, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes, oils such as peanut oil, cottonseed oil; safflower oil; sesame oil; olive oil; corn oil and soybean oil; glycols such a propylene glycol or polyethylene glycol; esters such as ethyl oleate and ethyl laurate, agar; buffering agents such as magnesium hydroxide and aluminum hydroxide; alginic acid; pyrogen-free water, isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The pharmaceutical compositions of this application may be administered to humans and other animals orally, rectally, parenterally, intracisternally, intravaginally, intraperitoneally, topically (as by powders, ointments, or drops), buccally, or as an oral or nasal spray.

Liquid dosage forms for oral administration may include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, com, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous, or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in tum, may depend upon crystal size and crystalline form. Alternatively, delayed absorption of a parenterally administered drug form is accomplished by dissolving or suspending the drug in an oil vehicle.

Compositions for rectal or vaginal administration are preferably suppositories which can be prepared by mixing the compounds of this application with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid compositions of a similar type may also be employed as fillers in soft and hard filled gelatin capsules using such excipients as lactose or milk sugar as well as high molecular weight polyethylene glycols and the like.

The active compounds may also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents.

Dosage forms for topical or transdermal administration of a compound of this application include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, eye ointments, powders and solutions are also contemplated as being within the scope of this application.

The ointments, pastes, creams and gels may contain, in addition to an active compound of this application, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compounds of this application, excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

For any compound, the therapeutically effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED50 (the dose therapeutically effective in 50% of the population) and LD50 (the dose lethal to 50% of the population). The dose ratio between toxic and therapeutic effects is the therapeutic index, and it can be expressed as the ratio, LD50/ED50.Pharmaceutical compositions that exhibit large therapeutic indices are preferred. The dosage may vary within this range depending upon the dosage form employed, sensitivity of the patient, and the route of administration.

Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

The quantity of active ingredient (e.g., a formulation of the disclosed compound or salt, hydrate, solvate or isomer thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this application include powders, sprays, ointments, pastes, creams, lotions, gels, solutions, patches and inhalants. In one embodiment, the active compound is mixed under sterile conditions with a pharmaceutically acceptable carrier, and with any preservatives, buffers or propellants that are required.

The pharmaceutical compositions containing active compounds of the present application may be manufactured in a manner that is generally known, e.g., by means of conventional mixing, dissolving, granulating, dragee-making, levigating, emulsifying, encapsulating, entrapping, or lyophilizing processes. Pharmaceutical compositions may be formulated in a conventional manner using one or more pharmaceutically acceptable carriers comprising excipients and/or auxiliaries that facilitate processing of the active compounds into preparations that can be used pharmaceutically. Of course, the appropriate formulation is dependent upon the route of administration chosen.

Techniques for formulation and administration of the disclosed compounds of the application can be found in Remington: the Science and Practice of Pharmacy, 19^{th} edition, Mack Publishing Co., Easton, PA (1995), which is herein expressly incorporated by reference in its entirety and for all purposes. In an embodiment, the compounds described herein, and the pharmaceutically acceptable salts thereof, are used in pharmaceutical preparations in combination with a pharmaceutically acceptable carrier or diluent. Suitable pharmaceutically acceptable carriers include inert solid fillers or diluents and sterile aqueous or organic solutions. The compounds will be present in such pharmaceutical compositions in amounts sufficient to provide the desired dosage amount in the range described herein.

Therapies based on the use CDNs as anti-tumor agents have been tested. In this scenario, CDNs are directly inoculated into tumors which plausibly stimulate APC activity to augment anti-tumor T cells responses. However, while working robustly in murine tumor models, the CDNs have exhibited little effect in human cancer trials, almost certainly due to their high turnover rate, in vivo. This has led to the generation of non-nucleotide-based STING agonists (small drugs), which may be able to escape degradation more effectively. STING signaling in the context of combined treatment with checkpoint inhibitors found that the therapeutic effect of an immune checkpoint inhibitory receptor (CTLA-4) and anti-PD-L1 monoclonal antibodies was lost in STING-deficient mice. In an embodiment of the present invention, STAVs represent a new generation of innate immune activators that trigger STING signaling.

Retrieved tumor cells transfected with STAVs activate APCs in trans and can generate potent anti-tumor T cell activity. Immunocompetent mice bearing metastatic syngeneic tumors can be treated with STAV ‘loaded’ tumor cells after reinfusion and inoculation. Select leukemias, such as acute myeloid leukemia (AML), acute lymphocytic leukemia (ALL) and adult T cell leukemia (ALL) can theoretically be amenable to treatment with STAVs. Further, the range of cancers can be extended to include melanomas and cutaneous T cell lymphomas. The intratumoral (I.T.) inoculation of syngeneic melanoma tumors (B16) in immunocompetent mice can be used to generate effective anti-tumor CTL activity and cause tumor regression. However, in situations where it is not feasible to retrieve sufficient tumor cells to carry out the transfection with STAVs for re-infusion, the STAV based approach may not be applicable.

In an embodiment of the present invention, the direct introduction of the STAVs into the tumor microenvironment (TME) can represent a significant advance. Further, in various embodiments of the present invention, the range of cancers amenable to STAV therapy can be extended using a non-cell based LNP strategy that effectively delivers high concentrations of Nano-STA Vs into the TME to potently generate anti-tumor cytotoxic T cell activity. In an embodiment of the present invention, the tumor regression generated by Nano-STAVs can be augmented by co-delivery of checkpoint inhibitors.

In an embodiment of the present invention, data indicates that Nano-STA Vs are a potent anti-tumor therapy that suppresses the growth of localized tumors (B16 melanoma model in C57/BL6 mice). In an embodiment of the present invention, the tumor regression effect was greatly augmented with the synergistic addition of checkpoint inhibitors. In an embodiment of the present invention, the activation of STING signaling in APC's is a main mechanism of generating anti-tumor T cell activity and is capable of overcoming resistance to checkpoint therapy. In an embodiment of the present invention, the benefit of Nano-STAVs over small drug agonists is that the procedure mimics the normal process of antigen cross-presentation, is non-toxic, simple, and inexpensive.

Potency can also be determined by IC₅₀ value. A compound with a lower IC50 value, as determined under substantially similar conditions, is more potent relative to a compound with a higher IC50 value. In some embodiments, the substantially similar conditions comprise determining the level of binding of a known STING ligand to a STING protein, in vitro or in vivo, in the presence of a compound of the application.

In one embodiment, the compounds of the present application are useful as therapeutic agents, and thus may be useful in the treatment of a disease caused by, or associated with, STING expression, activity, and/or function (e.g., deregulation of STING expression, activity, and/or function) or a disease associated with one or more of the intracellular pathways that STING is involved in (e.g. regulation of intracellular DNA-mediated type I interferon activation), such as those described herein.

A ‘selective STING modulator’ can be identified, for example, by comparing the ability of a compound to modulate STING expression/activity/function to its ability to modulate the other proteins or a STING protein from another species. In some embodiments, the selectivity can be identified by measuring the EC₅₀ or IC₅₀ of the compounds.

The compounds of the application are defined herein by their chemical structures and/or chemical names. Where a compound is referred to by both a chemical structure and a chemical name, and the chemical structure and chemical name conflict, the chemical structure is determinative of the compound's identity.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof.

In another aspect, the application provides a method of synthesizing a compound disclosed herein. The synthesis of the compounds of the application can be found herein and in the Examples below. Other embodiments are a method of making a compound of any of the formulae herein using any one, or combination of, reactions delineated herein. The method can include the use of one or more intermediates or chemical reagents delineated herein.

The application also provides for a pharmaceutical composition comprising a therapeutically effective amount of a compound of the application, or a pharmaceutically acceptable salt or ester thereof, and a pharmaceutically acceptable carrier.

Another aspect of the present application relates to a kit comprising a compound of the application or a pharmaceutically acceptable salt or ester thereof, or a pharmaceutical composition of the application. In another aspect, the application provides a kit comprising a compound capable of modulating STING activity selected from one or more compounds disclosed herein, or a pharmaceutically acceptable salt or ester thereof, optionally in combination with a second agent and instructions for use.

Another aspect of the present application relates to a compound of the application or a pharmaceutically acceptable salt or ester thereof, or a pharmaceutical composition of the application, for use in the manufacture of a medicament for modulating (e.g., inhibiting or stimulating) a STING protein, for treating or preventing a disease, wherein the diseases is caused by, or associated with, STING expression, activity, and/or function (e.g., deregulation of STING expression, activity, and/or function), or for treating or preventing a disease associated with deregulation of one or more of the intracellular pathways in which a STING protein is involved (e.g., deregulation of intracellular dsDNA mediated type I interferon activation).

Another aspect of the present application relates to use of a compound of the application or a pharmaceutically acceptable salt or ester thereof, or a pharmaceutical composition of the application, in the manufacture of a medicament for modulating (e.g., inhibiting or stimulating) a STING protein, for treating or preventing a disease, wherein the diseases is caused by, or associated with, STING expression, activity, and/or function (e.g., deregulation of STING expression, activity, and/or function), or for treating or preventing a disease associated with deregulation of one or more of the intracellular pathways in which a STING protein is involved (e.g., deregulation of intracellular dsDNA mediated type I interferon activation).

Another aspect of the present application relates to a compound of the application or a pharmaceutically acceptable salt or ester thereof, or a pharmaceutical composition of the application, for use in modulating (e.g., inhibiting or stimulating) a STING protein, in treating or preventing a disease, wherein the diseases is caused by, or associated with, STING expression, activity, and/or function (e.g., deregulation of STING expression, activity, and/or function), or in treating or preventing a disease associated with deregulation of one or more of the intracellular pathways in which a STING protein is involved (e.g., deregulation of intracellular dsDNA mediated type I interferon activation).

Another aspect of the present application relates to use of a compound of the application or a pharmaceutically acceptable salt or ester thereof, or a pharmaceutical composition of the application, in antagonizing a STING protein, in treating or preventing a disease, wherein the diseases is caused by, or associated with, STING expression, activity, and/or function (e.g., deregulation of STING expression, activity, and/or function), or in treating or preventing a disease associated with deregulation of one or more of the intracellular pathways in which a STING protein is involved (e.g., deregulation of intracellular dsDNA mediated type I interferon activation).

### Method of Synthesizing the Compounds

Compounds of the present application can be prepared in a variety of ways using commercially available starting materials, compounds known in the literature, or from readily prepared intermediates, by employing standard synthetic methods and procedures either known to those skilled in the art, or which will be apparent to the skilled artisan in light of the teachings herein. Standard synthetic methods and procedures for the preparation of organic molecules and functional group transformations and manipulations can be obtained from the relevant scientific literature or from standard textbooks in the field. Although not limited to any one or several sources, classic texts such as Smith, M. B., March, J., March's Advanced Organic Chemistry: Reactions, Mechanisms, and Structure, 5^{th} edition, John Wiley & Sons: New York, 2001; and Greene, T. W., Wuts, P. G. M., Protective Groups in Organic Synthesis, 3^{rd} edition, John Wiley & Sons: New York, 1999 are useful and recognized reference textbooks of organic synthesis known to those in the art. The following descriptions of synthetic methods are designed to illustrate, but not to limit, general procedures for the preparation of compounds of the present application. The processes generally provide the desired final compound at or near the end of the overall process, although it may be desirable in certain instances to further convert the compound to a pharmaceutically acceptable salt, ester, or prodrug thereof. Suitable synthetic routes are depicted in the schemes below.

### Example: Synthesis and Evaluation of STAVs

In an embodiment of the present invention, a variety of ssDNA and dsDNA oligonucleotides, containing exonuclease resistant phosphorothioates at the ends(ES) that varied in their nucleotide content were synthesized (clinical grade, TriLink Biotechnologies) using procedures known to a person of ordinary skill in the art. The ssDNA and dsDNA oligonucleotides evaluated to determine which was better at stimulating STING signaling following transfection of normal human and mouse cells including APCs had nucleotide content as follows:

$$A : T 30 ES = polyA 30 ES ( SEQ ID NO : 1 ) + polyT 30 ES ( SEQ ID NO : 2 )$$

polyA30ES
(SEQ ID NO: 1)
A(ps)A(ps)A(ps)AAAAAAAAAAAAAAAAAAAAAAAA(ps)A(ps)A(ps)A
polyT30E
(SEQ ID NO: 2)
T(ps)T(ps)T(ps)TTTTTTTTTTTTTTTTTTTTTTTT(ps)T(ps)T(ps)T
A: T50ES = polyA50ES (SEQ ID NO: 3) + polyT50ES (SEQ ID NO: 4)
poly A50ES
(SEQ ID NO: 3)
A(ps)A(ps)A(ps)AAAAAAAAAAAAAAAAAAAAAAAAAAAAAAAAAAAAAAAAAAAA
(ps)A(ps)A(ps)A
polyT50ES
(SEQ ID NO: 4)
T(ps)T(ps)T(ps)TTTTTTTTTTTTTTTTTTTTTTTTTTTTTTTTTTTTTTTTTTTT(ps)T(ps)T(ps)
T
A: T60ES = polyA60ES (SEQ ID NO: 5) + polyT60ES (SEQ ID NO: 6)
poly A60ES
(SEQ ID NO: 5)
A(ps)A(ps)A(ps)AAAAAAAAAAAAAAAAAAAAAAAAAAAAAAAAAAAAAAAAAAAA
AAAAAAAAAA(ps)A(ps)A(ps)A
polyT60ES
(SEQ ID NO: 6)
T(ps)T(ps)T(ps)TTTTTTTTTTTTTTTTTTTTTTTTTTTTTTTTTTTTTTTTTTTTTTTTTTTTT
T(ps)T(ps)T(ps)T
A: T70ES = poly A70ES (SEQ ID NO: 7) + polyT70ES (SEQ ID NO: 8)
polyA70ES
(SEQ ID NO: 7)
A(ps)A(ps)A(ps)AAAAAAAAAAAAAAAAAAAAAAAAAAAAAAAAAAAAAAAAAAAA
AAAAAAAAAAAAAAAAAAAA(ps)A(ps)A(ps)A
polyT70ES
(SEQ ID NO: 8)
T(ps)T(ps)T(ps)TTTTTTTTTTTTTTTTTTTTTTTTTTTTTTTTTTTTTTTTTTTTTTTTTTTTT
TTTTTTTTTTT(ps)T(ps)T(ps)T
A: T80ES = poly A80ES (SEQ ID NO: 9) + polyT80ES (SEQ ID NO: 10)
polyA80ES
(SEQ ID NO: 9)
A(ps)A(ps)A(ps)AAAAAAAAAAAAAAAAAAAAAAAAAAAAAAAAAAAAAAAAAAAA
AAAAAAAAAAAAAAAAAAAAAAAAAAAAAA(ps)A(ps)A(ps)A
polyT80ES
(SEQ ID NO: 10)
T(ps)T(ps)T(ps)TTTTTTTTTTTTTTTTTTTTTTTTTTTTTTTTTTTTTTTTTTTTTTTTTTTTT
TTTTTTTTTTTTTTTTTTTTT(ps)T(ps)T(ps)T
A: T90ES = polyA90ES (SEQ ID NO: 11) + polyT90ES (SEQ ID NO: 12)
poly A90ES
(SEQ ID NO: 11)
A(ps)A(ps)A(ps)AAAAAAAAAAAAAAAAAAAAAAAAAAAAAAAAAAAAAAAAAAAA
AAAAAAAAAAAAAAAAAAAAAAAAAAAAAAAAAAAAAAAA(ps)A(ps)A(ps)A
polyT90ES
(SEQ ID NO: 12)
T(ps)T(ps)T(ps)TTTTTTTTTTTTTTTTTTTTTTTTTTTTTTTTTTTTTTTTTTTTTTTTTTTTT
TTTTTTTTTTTTTTTTTTTTTTTTTTTTTTT(ps)T(ps)T(ps)T
A: T100ES = polyA100ES (SEQ ID NO: 13) + polyT100ES (SEQ ID NO: 14)
poly A100ES
(SEQ ID NO: 13)
A(ps)A(ps)A(ps)AAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAA(ps)A
(ps)A(ps)A
polyT100ES
(SEQ ID NO: 14)
T(ps)T(ps)T(ps)TTTTTTTTTTTTTTTTTTTTTTTTTTTTTTTTTTTTTTTTTTTTTTTTTTTTT
TTTTTTTTTTTTTTTTTTTTTTTTTTTTTTTTTTTTTTTTT(ps)T(ps)T(ps)T
A: T110ES = polyA110ES (SEQ ID NO: 15) + polyT110ES (SEQ ID NO: 16)
(SEQ ID NO: 15)
polyA110ES A(ps)A(ps)A(ps)AAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAAAA
AAAAAAAAAA(ps)A(ps)A(ps)A
(SEQ ID NO: 16)
polyT110EST(ps)T(ps)T(ps)TTTTTTTTTTTTTTTTTTTTTTTTTTTTTTTTTTTTTTTTTTTTTTTTTTT
TTTTTTTTTTTTTTTTTTTTTTTTTTTTTTTTTTTTTTTTTTTTTTTTTTTTT(ps)T(ps)T(ps)T
(SEQ ID NO: 17)
GC30ES G(ps)C(ps)G(ps)CGCGCGCGCGCGCGCGCGCGCGCG(ps)C(ps)G(ps)C
GC50ES
(SEQ ID NO: 18)
G(ps)C(ps)G(ps)CGCGCGCGCGCGCGCGCGCGCGCGCGCGCGCGCGCGCGCGCGCG(ps)
C(ps)G(ps)C
GC60ES
(SEQ ID NO: 19)
G(ps)C(ps)G(ps)CGCGCGCGCGCGCGCGCGCGCGCGCGCGCGCGCGCGCGCGCGCGCG
CGCGCGCG(ps)C(ps)G(ps)C
GC70ES
(SEQ ID NO: 20)
G(ps)C(ps)G(ps)CGCGCGCGCGCGCGCGCGCGCGCGCGCGCGCGCGCGCGCGCGCGCG
CGCGCGCGCGCGCGCGCG(ps)C(ps)G(ps)C
GC80ES
(SEQ ID NO: 21)
G(ps)C(ps)G(ps)CGCGCGCGCGCGCGCGCGCGCGCGCGCGCGCGCGCGCGCGCGCGCG
CGCGCGCGCGCGCGCGCGCGCGCGCGCG(ps)C(ps)G(ps)C
GC90ES
(SEQ ID NO: 22)
G(ps)C(ps)G(ps)CGCGCGCGCGCGCGCGCGCGCGCGCGCGCGCGCGCGCGCGCGCGCG
CGCGCGCGCGCGCGCGCGCGCGCGCGCGCGCGCGCGCG(ps)C(ps)G(ps)C
GC100ES
(SEQ ID NO: 23)
G(ps)C(ps)G(ps)CGCGCGCGCGCGCGCGCGCGCGCGCGCGCGCGCGCGCGCGCGCGCG
CGCGCGCGCGCGCGCGCGCGCGCGCGCGCGCGCGCGCGCGCGCGCGCG(ps)C(ps)G(ps)
C
STAV1 (A:T76ES) = polyA76ES (SEQ ID NO: 24) + polyT76ES (SEQ ID NO: 25)
PolyA76ES
(SEQ ID NO: 24)
A(ps)A(ps)A(ps)AAAAAAAAAAAAAAAAAAAAAAAAAAAAAAAAAAAAAAAAAAAA
AAAAAAAAAAAAAAAAAAAAAAAAAA(ps)A(ps)A(ps)A
PolyT76ES
(SEQ ID NO: 25)
T(ps)T(ps)T(ps)TTTTTTTTTTTTTTTTTTTTTTTTTTTTTTTTTTTTTTTTTTTTTTTTTTTTT
TTTTTTTTTTTTTTTTT(ps)T(ps)T(ps)T
STAV2 (PolyAC:TG76ES) = polyAC76ES (SEQ ID NO:26) + polyTG76ES (SEQ ID NO: 27)
PolyAC76ES
(SEQ ID NO: 26)
A(ps)C(ps)A(ps)CACACACACACACACACACACACACACACACACACACACACACACA
CACACACACACACACACACACACA(ps)C(ps)A(ps)C
PolyTG76ES
(SEQ ID NO: 27)
G(ps)T(ps)G(ps)TGTGTGTGTGTGTGTGTGTGTGTGTGTGTGTGTGTGTGTGTGTGTGTG
TGTGTGTGTGTGTGTGTGTGTG(ps)T(ps)G(ps)T
PolyAT76ES
(SEQ ID NO: 28)
A(ps)T(ps)A(ps)TATATATATATATATATATATATATATATATATATATATATATATATA
TATATATATATATATATATATA(ps)T(ps)A(ps)T
PolyTA76ES
(SEQ ID NO: 29)
T(ps)A(ps)T(ps)ATATATATATATATATATATATATATATATATATATATATATATATAT
ATATATATATATATATATATAT(ps)A(ps)T(ps)A
PolyACTG76 ES
(SEQ ID NO: 30)
A(ps)C(ps)T(ps)GACTGACTGACTGACTGACTGACTGACTGACTGACTGACTGACTGAC
TGACTGACTGACTGACTGACTGA(ps)C(ps)T(ps)G
PolyCAGT76 ES
(SEQ ID NO: 31)
C(ps)A(ps)G(ps)TCAGTCAGTCAGTCAGTCAGTCAGTCAGTCAGTCAGTCAGTCAGTCA
GTCAGTCAGTCAGTCAGTCAGTC(ps)A(ps)G(ps)T
HSV RL2 intron-S
(SEQ ID NO: 32)
G(ps)A(ps)C(ps)CCTATCGATACAGGGCACGGGGTCGAACTGTTGGGTTTCGCCATGGT
ACCCCCTGCATTTATATAGCCAG(ps)A(ps)C(ps)C
HSV RL2 intron-AS
(SEQ ID NO: 33)
G(ps)G(ps)T(ps)CTGGCTATATAAATGCAGGGGGTACCATGGCGAAACCCAACAGTTC
GACCCCGTGCCCTGTATCGATAGG(ps)G(ps)T(ps)C
polyA90ES-FAM FAM-
(SEQ ID NO: 35)
A(ps)A(ps)A(ps)AAAAAAAAAAAAAAAAAAAAAAAAAAAAAAAAAAAAAAAAAAAA
AAAAAAAAAAAAAAAAAAAAAAAAAAAAAAAAAAAAAAAA(ps)A(ps)A(ps)A
polyT90ES FAM-
(SEQ ID NO: 36)
T(ps)T(ps)T(ps)TTTTTTTTTTTTTTTTTTTTTTTTTTTTTTTTTTTTTTTTTTTTTTTTTTTTT
TTTTTTTTTTTTTTTTTTTTTTTTTTTTTTT(ps)T(ps)T(ps)T
STAV3 (AT:TA76 ES) = polyAT76ES (SEQ ID NO: 37) + polyTA76ES (SEQ ID NO: 38)
PolyAT:TA76ES
(SEQ ID NO: 37)
A(ps)T(ps)T(ps)AATTAATTAATTAATTAATTAATTAATTAATTAATTAATTAATTAATT
AATTAATTAATTAATTAATTAA(ps)T(ps)T(ps)A
PolyTA:AT76ES
(SEQ ID NO: 38)
T(ps)A(ps)A(ps)TTAATTAATTAATTAATTAATTAATTAATTAATTAATTAATTAATTAA
TTAATTAATTAATTAATTAATT(ps)A(ps)A(ps)T.
STAV4 = (SEQ ID NO: 39) + (SEQ ID NO: 40)
(SEQ ID NO: 39)
A(ps)C(ps)T(ps)GACTGACTGACTGACTGACTGACTGACTGACTGACTGACTGACTGAC
TGACTGACTGACTGACTGACTGA(ps)C(ps)T(ps)G
(SEQ ID NO: 40)
C(ps)A(ps)G(ps)TCAGTCAGTCAGTCAGTCAGTCAGTCAGTCAGTCAGTCAGTCAGTCA
GTCAGTCAGTCAGTCAGTCAGTC(ps)A(ps)G(ps)T.
STAV5 = (SEQ ID NO: 41) + (SEQ ID NO: 42)
(SEQ ID NO: 41)
G(ps)A(ps)C(ps)CCTATCGATACAGGGCACGGGGTCGAACTGTTGGGTTTCGCCATGGT
ACCCCCTGCATTTATATAGCCAG(ps)A(ps)C(ps)C
(SEQ ID NO: 42)
G(ps)G(ps)T(ps)CTGGCTATATAAATGCAGGGGGTACCATGGCGAAACCCAACAGTTC
GACCCCGTGCCCTGTATCGATAGG(ps)G(ps)T(ps)C.
STAV6 = (SEQ ID NO: 43) + (SEQ ID NO: 44)
(SEQ ID NO: 43)
A(ps)A(ps)A(ps)AAAAAAAAAAAAAAAAAAAAAAAAAAAAAAAAAAAAAAAAAAAA
AAAAAAAAAAAAAAAAAAAAAAAAAAAAAAAAAAAAAAAA(ps)A(ps)A(ps)A
(SEQ ID NO: 44)
T(ps)T(ps)T(ps)TTTTTTTTTTTTTTTTTTTTTTTTTTTTTTTTTTTTTTTTTTTTTTTTTTTTT
TTTTTTTTTTTTTTTTTTTTTTTTTTTTTTT(ps)T(ps)T(ps)T.
STAV7 = (SEQ ID NO: 45) + (SEQ ID NO: 46)
(SEQ ID NO: 45)
G(ps)C(ps)G(ps)CGCGCGCGCGCGCGCGCGCGCGCGCGCGCGCGCGCGCGCGCGCGCG
CGCGCGCGCGCGCGCGCGCGCGCGCGCGCGCGCGCGCG(ps)C(ps)G(ps)C
(SEQ ID NO: 46)
C(ps)G(ps)C(ps)GCGCGCGCGCGCGCGCGCGCGCGCGCGCGCGCGCGCGCGCGCGCGC
GCGCGCGCGCGCGCGCGCGCGCGCGCGCGCGCGCGCGC(ps)G(ps)C(ps)G.

A number of STAVs that were greater than 70 bp were effective in stimulating STING-based cytokine production. Based on the result that STAVs greater than 70 bp were effective in stimulating STING, three (3) STAVs were used herein as follows: STAV1 a double-stranded polyA:T76ES, oligonucleotides (SEQ ID NO:24)+(SEQ ID NO:25); STAV2 a double-stranded polyAC:TG76ES, oligonucleotides (SEQ ID NO:26)+(SEQ ID NO:27); and STAV3 a double-stranded polyAT:TA76ES, oligonucleotides (SEQ ID NO:37)+(SEQ ID NO:38).

### Nano-STAV Synthesis

In an embodiment of the present invention, a LNP comprising distearoylphosphatidylcholine, cholesterol, MC3, and DMG-PEG 2000 is dissolved in ethanol that is rapidly mixed with the STAV1-3 (SEQ ID NO:24)+(SEQ ID NO:25); STAV2 a double-stranded polyAC:TG76ES, oligonucleotides (SEQ ID NO: 26)+(SEQ ID NO:27); and STAV3 a double-stranded polyAT:TA76ES, oligonucleotides (SEQ ID NO: 37)+(SEQ ID NO:38) in aqueous buffer at a pH where the ionizable lipid is positively charged (pH approximately 4, where approximately means+−pH 1). The resulting dispersion is then dialyzed against a normal saline buffer to remove residual ethanol and raise the pH above the pKa of the cationic lipid (pH approximately 7.4, where approximately means+−pH 0.5) to produce the finished Nano-STAV1, Nano-STAV 2, and Nano-STAV3.

### Check Point Inhibitor Analysis

Anti-PD-L1 (IgG BE0091 or anti-PD-L1 BE0101, BioXcell) and anti-PD1 (J43 BE0033-2, BioXcell) were used in the B16 melanoma model. Sex matched C57/BL6 mice (n=10) were inoculated with B16-OVA (5×10⁵) on the flanks. After 7, 10, and 13 days, when tumors are 50 mm³ in volume, 25 μl (4 μg/mL; 0.1 μg/mouse) of Nano-STAVs (STAV1 a double-stranded polyA: T76ES, oligonucleotides (SEQ ID NO:24)+(SEQ ID NO:25); STAV2 a double-stranded polyAC:TG76ES, oligonucleotides (SEQ ID NO: 26)+(SEQ ID NO:27); and STAV3 a double-stranded polyAT:TA76ES, oligonucleotides (SEQ ID NO: 37)+(SEQ ID NO:38)) were injected i.t. in presence or absence of anti-PD-1 or anti-PD-L1 (50μg/mouse).

The compounds of the present application can be prepared in a number of ways well known to those skilled in the art of organic synthesis. By way of example, compounds of the present application can be synthesized using the methods described below, together with synthetic methods known in the art of synthetic organic chemistry, or variations thereon as appreciated by those skilled in the art. Preferred methods include but are not limited to those methods described below.

Compounds of the present application can be synthesized by following the steps outlined in the following Schemes, which comprise different sequences of assembling intermediates. Starting materials are either commercially available or made by known procedures in the reported literature or as illustrated.

A compound of the application can be prepared as a pharmaceutically acceptable acid addition salt by reacting the free base form of the compound with a pharmaceutically acceptable inorganic or organic acid. Alternatively, a pharmaceutically acceptable base addition salt of a compound of the application can be prepared by reacting the free acid form of the compound with a pharmaceutically acceptable inorganic or organic base. The pharmaceutically acceptable salt may include various counterions, e.g., counterions of the inorganic or organic acid, counterions of the inorganic or organic base, or counterions afforded by counterion exchange.

Acids and bases useful in the methods herein are known in the art. Acid catalysts are any acidic chemical, which can be inorganic (e.g., hydrochloric, sulfuric, nitric acids, aluminum trichloride) or organic (e.g., camphorsulfonic acid, p-toluenesulfonic acid, acetic acid, ytterbium triflate) in nature. Acids are useful in either catalytic or stoichiometric amounts to facilitate chemical reactions. Bases are any basic chemical, which can be inorganic (e.g., sodium bicarbonate, potassium hydroxide) or organic (e.g., triethylamine, pyridine) in nature. Bases are useful in either catalytic or stoichiometric amounts to facilitate chemical reactions.

Alternatively, the salt forms of the compounds of the application can be prepared using salts of the starting materials or intermediates. The free acid or free base forms of the compounds of the application can be prepared from the corresponding base addition salt or acid addition salt from, respectively. For example, a compound of the application in an acid addition salt form can be converted to the corresponding free base by treating with a suitable base (e.g., ammonium hydroxide solution, sodium hydroxide, and the like). A compound of the application in a base addition salt form can be converted to the corresponding free acid by treating with a suitable acid (e.g., hydrochloric acid, etc.).

Those skilled in the art will recognize if a stereo center exists in the compounds disclosed herein. Accordingly, the present application includes both possible stereoisomers (unless specified in the synthesis) and includes not only racemic compounds but the individual enantiomers and/or diastereomers as well. When a compound is desired as a single enantiomer or diastereomer, it may be obtained by stereospecific synthesis or by resolution of the final product or any convenient intermediate. Resolution of the final product, an intermediate, or a starting material may be affected by any suitable method known in the art. See, for example, “Stereochemistry of Organic Compounds” by E. L. Eliel, S. H. Wilen, and L. N. Mander (Wiley-Interscience, 1994).

The synthesized compounds can be separated from a reaction mixture and further purified by a method such as column chromatography, high pressure liquid chromatography, or recrystallization. As can be appreciated by the skilled artisan, further methods of synthesizing the compounds of the formulae herein will be evident to those of ordinary skill in the art. Additionally, the various synthetic steps may be performed in an alternate sequence or order to give the desired compounds. In addition, the solvents, temperatures, reaction durations, etc. delineated herein are for purposes of illustration only and one of ordinary skill in the art will recognize that variation of the reaction conditions can produce the desired bridged macrocyclic products of the present application. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), which are herein expressly incorporated by reference in their entireties and for all purposes, and subsequent editions thereof.

In one aspect, the present application provides a method of inhibiting a STING protein. The method comprises administering to a subject in need thereof an effective amount of a compound of the application or a pharmaceutically acceptable salt or ester thereof, or a pharmaceutical composition of the application.

In some embodiments, the modulation of a STING protein activity is measured by IC₅₀. In some embodiments, the modulation of a STING protein activity is measured by EC₅₀.

A compound of the present application (e.g., a compound of any of the formulae described herein, or selected from any compounds described herein) is capable of treating or preventing a disease, wherein the diseases is caused by, or associated with, STING expression, activity, and/or function (e.g., deregulation of STING expression, activity, and/or function) or a disease associated with deregulation of one or more of the intracellular pathways in which a STING protein is involved (e.g., deregulation of intracellular dsDNA mediated type I interferon activation).

In one aspect, the present application provides a method of treating or preventing a disease, wherein the diseases is caused by, or associated with, STING expression, activity, and/or function (e.g., deregulation of STING expression, activity, and/or function). The method comprises administering to a subject in need thereof an effective amount of a STING antagonist compound of the application or a pharmaceutically acceptable salt or ester thereof, or a pharmaceutical composition of the application. In one aspect, the disease is a STING mediated disorder.

In one aspect, the present application provides a method of treating or preventing a disease associated with deregulation of one or more of the intracellular pathways in which a STING protein is involved (e.g., deregulation of intracellular dsDNA mediated type I interferon activation). The method comprises administering to a subject in need thereof an effective amount of a STING antagonist compound of the application or a pharmaceutically acceptable salt or ester thereof, or a pharmaceutical composition of the application.

In one embodiment, the present application provides a method of treating or preventing any of the diseases, disorders, and conditions described herein, wherein the subject is a human. In one embodiment, the application provides a method of treating. In one embodiment, the application provides a method of preventing.

As antagonists of a STING protein, the compounds and compositions of this application are particularly useful for treating or lessening the severity of a disease, condition, or disorder where a STING protein or one or more of the intracellular pathways that STING is involved is implicated in the disease, condition, or disorder. In one embodiment, the present application provides a method for treating or lessening the severity of a disease, condition, or disorder with STING antagonist compounds that modulate binding of a cyclic di-nucleotide. (CDN) including non-canonical cyclic di-nucleotide, such as 2′3′cGAMP, to a STING protein. In one embodiment, the present application provides a method for treating or lessening the severity of a disease, condition, or disorder with compounds that modulate the synthesis of type I interferon and/or type I IFN response and other cytokines, chemokines (STING-inducible proteins).

In one aspect, the present application also provides a method of treating or preventing cell proliferative disorders such as hyperplasias, dysplasias, or pre-cancerous lesions. Dysplasia is the earliest form of pre-cancerous lesion recognizable in a biopsy by a pathologist. The compounds of the present application may be administered for the purpose of preventing hyperplasias, dysplasias, or pre-cancerous lesions from continuing to expand or from becoming cancerous. Examples of pre-cancerous lesions may occur in skin, esophageal tissue, breast, and cervical intra-epithelial tissue.

In one embodiment, the disease or disorder includes, but is not limited to, immune disorders, autoimmunity, a cell proliferative disease or disorder, cancer, inflammation, graft vs host, transplantation, gastrointestinal disorder, rheumatoid arthritis, systemic lupus, cachexia, neurodegenerative disease or disorders, neurological diseases or disorders, cardiac dysfunction, or microbial infection (e.g., viral, bacterial, and/or fungi infection, parasitic, or infection caused by other microorganism).

In one embodiment, the disease or disorder is a cell proliferative disease or disorder.

As used herein, the term ‘cell proliferative disorder’ refers to conditions in which unregulated or abnormal growth, or both, of cells can lead to the development of an unwanted condition or disease, which may or may not be cancerous. Exemplary cell proliferative diseases or disorders encompass a variety of conditions wherein cell division is deregulated. Exemplary cell proliferative disorders include, but are not limited to, neoplasms, benign tumors, malignant tumors, pre-cancerous conditions, in situ tumors, encapsulated tumors, metastatic tumors, liquid tumors, solid tumors, immunological tumors, hematological tumors, cancers, carcinomas, leukemias, lymphomas, sarcomas, and rapidly dividing cells. The term ‘rapidly dividing cell’ as used herein is defined as any cell that divides at a rate that exceeds or is greater than what is expected or observed among neighboring or juxtaposed cells within the same tissue. A cell proliferative disease or disorder includes a precancer or a precancerous condition. A cell proliferative disease or disorder includes cancer.

In one embodiment, the proliferative disease or disorder is a non-cancerous. In one embodiment, the non-cancerous disease or disorder includes, but is not limited to, rheumatoid arthritis; inflammation; autoimmune disease; lymphoproliferative conditions; acromegaly; rheumatoid spondylitis; osteoarthritis; gout; other arthritic conditions; sepsis; septic shock; endotoxic shock; gram-negative sepsis; toxic shock syndrome; asthma; adult respiratory distress syndrome; chronic obstructive pulmonary disease; chronic pulmonary inflammation; inflammatory bowel disease; Crohn's disease; skin-related hyperproliferative disorders; psoriasis; eczema; atopic dermatitis; hyperpigmentation disorders; eye-related hyperproliferative disorders; age-related macular degeneration; ulcerative colitis; pancreatic fibrosis; hepatic fibrosis; acute and chronic renal disease; irritable bowel syndrome; pyresis; restenosis; cerebral malaria; stroke and ischemic injury; neural trauma; Alzheimer's disease; Huntington's disease; Parkinson's disease; acute and chronic pain; allergic rhinitis; allergic conjunctivitis; chronic heart failure; acute coronary syndrome; cachexia; malaria; leprosy; leishmaniasis; Lyme disease; Reiter's syndrome; acute synovitis; muscle degeneration, bursitis; tendonitis; tenosynovitis; herniated, ruptures, or prolapsed intervertebral disk syndrome; osteopetrosis; thrombosis; restenosis; silicosis; pulmonary sarcosis; bone resorption diseases, such as osteoporosis; graft-versus-host reaction; fibroadipose hyperplasia; spinocerebullar ataxia type 1; CLOVES syndrome; Harlequin ichthyosis; macrodactyly syndrome; Proteus syndrome (Wiedemann syndrome); LEOPARD syndrome; systemic sclerosis; Multiple Sclerosis; lupus; fibromyalgia; AIDS and other viral diseases such as Herpes Zoster, Herpes Simplex I or II, influenza virus and cytomegalovirus; diabetes mellitus; hemihyperplasia-multiple lipomatosis syndrome; megalencephaly; rare hypoglycemia, Klippel-Trenaunay syndrome; harmatoma; Cowden syndrome; or overgrowth-hyperglycemia.

In one embodiment, the proliferative disease or disorder is cancer. In one embodiment, the cancer is lung cancer, colon cancer, breast cancer, prostate cancer, liver cancer, pancreas cancer, brain cancer, kidney cancer, ovarian cancer, stomach cancer, skin cancer, bone cancer, gastric cancer, breast cancer, pancreatic cancer, glioma, glioblastoma, hepatocellular carcinoma, papillary renal carcinoma, head and neck squamous cell carcinoma, leukemias, lymphomas, myelomas, or solid tumors.

The term ‘cancer’ refers to any disease caused by the proliferation of malignant neoplastic cells. such as tumors, neoplasms, carcinomas, sarcomas, leukemias, lymphomas and the like. The term ‘cancer’ includes, but is not limited to, the following cancers: breast, myeloma, lymphoma, ovary, cervix, prostate, testis, genitourinary tract, esophagus, larynx, glioblastoma, neuroblastoma, stomach, skin, keratoacanthoma, lung, epidermoid carcinoma, large cell carcinoma, small cell carcinoma, lung adenocarcinoma, bone, colon, colorectal, adenoma, pancreas, thyroid, follicular carcinoma, undifferentiated carcinoma, papillary carcinoma, seminoma, melanoma, sarcoma, liver carcinoma and biliary passages, kidney carcinoma, myeloid disorders, lymphoid disorders, Hodgkin's, hairy cells, buccal cavity and pharynx (oral), lip, tongue, mouth, pharynx, small intestine, rectum, large intestine, brain and central nervous system, chronic myeloid leukemia (CML), or a cancer selected from gastric, renal, head and neck, oropharangeal, non-small cell lung cancer (NSCLC), endometrial, hepatocarcinoma, Non-Hodgkins lymphoma, and pulmonary, epidermoid Oral: buccal cavity, Cardiac: sarcoma (angiosarcoma, fibrosarcoma, rhabdomyosarcoma, liposarcoma), myxoma, rhabdomyoma, fibroma, lipoma, and teratoma, Lung: bronchogenic carcinoma (squamous cell or epidermoid, undifferentiated small cell, undifferentiated large cell, adenocarcinoma), alveolar (bronchiolar) carcinoma, bronchial adenoma, sarcoma, lymphoma, chondromatous hamartoma, mesothelioma, Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel or small intestines (adenocarcinoma, lymphoma, leiomyoma, hemangioma, lipoma, neurofibroma), large bowel or large intestines (adenocarcinoma, tubular adenoma, villous adenoma, hamartoma, leiomyoma), rectum, Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroadenoma, adenomatoid tumors, lipoma), Liver: hepatoma (hepatocellular carcinoma), cholangiocarcinoma, hepatoblastoma, angiosarcoma, hepatocellular adenoma, biliary passages, Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors, Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pincaloma), glioblastoma multiform, oligodendroglioma, schwannoma, retinoblastoma, congenital tumors), spinal cord neurofibroma, glioma, sarcoma), Gynecological: uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma), Hematologic: blood (myeloid leukemia (acute and chronic), acute lymphoblastic leukemia, chronic lymphocytic leukemia, myeloproliferative diseases, myelodysplastic syndrome), Hodgkin's disease, non-Hodgkin's lymphoma (malignant lymphoma) hairy cell, lymphoid disorders, Skin: malignant melanoma, basal cell carcinoma, squamous cell carcinoma, Karposi's sarcoma, moles dysplastic nevi, lipoma, angioma, dermatofibroma, keloids, psoriasis, Thyroid gland: papillary thyroid carcinoma, follicular thyroid carcinoma; medullary thyroid carcinoma, undifferentiated thyroid cancer, multiple endocrine neoplasia type 2A, multiple endocrine neoplasia type 2B, familial medullary thyroid cancer, pheochromocytoma, paraganglioma; and adrenal glands: neuroblastoma, leukemias and lymphomas such as cutaneous T-cell lymphomas (CTCL), noncutaneous peripheral T-cell lymphomas, lymphomas associated with human T-cell lymphotrophic virus (HTLV) such as adult T-cell leukemia/lymphoma (ATLL), B-cell lymphoma, acute nonlymphocytic leukemias, chronic myelogenous leukemia, acute myelogenous leukemia, lymphomas, non-Hodgkin lymphoma, acute lymphatic leukemia (ALL), chronic lymphatic leukemia (CLL), Hodgkin's lymphoma, Burkitt lymphoma, adult T-cell leukemia lymphoma, acute-myeloid leukemia (AML), or hepatocellular carcinoma. Further examples include myelodisplastic syndrome, childhood solid tumors such as brain tumors, retinoblastoma, Wilms' tumor, and soft-tissue sarcomas, common solid tumors of adults such as head and neck cancers (e.g., oral, laryngeal, nasopharyngeal and esophageal), genitourinary cancers (e.g., prostate, bladder, renal, uterine, ovarian, testicular), lung cancer (e.g., small-cell and non-small cell), pancreatic cancer, and other skin cancers, stomach cancer, tumors related to Gorlin's syndrome (e.g., medulloblastoma, meningioma, etc.), and liver cancer. Additional exemplary forms of cancer which may be treated include, but are not limited to, cancer of skeletal or smooth muscle, stomach cancer, cancer of the small intestine, rectum carcinoma, cancer of the salivary gland, endometrial cancer, adrenal cancer, anal cancer, rectal cancer, parathyroid cancer, and pituitary cancer. Cancer may also include colon carcinoma, familial adenomatous polyposis carcinoma or hereditary non-polyposis colorectal cancer. Further, cancers include, but are not limited to, labial carcinoma, larynx carcinoma, hypopharynx carcinoma, tongue carcinoma, salivary gland carcinoma, gastric carcinoma, adenocarcinoma, thyroid cancer (medullary and papillary thyroid carcinoma), renal carcinoma, kidney parenchyma carcinoma, uterine corpus carcinoma, endometrium carcinoma, chorion carcinoma, testis carcinoma, urinary carcinoma, brain tumors such as glioblastoma, astrocytoma, medulloblastoma and peripheral neuroectodermal tumors, gall bladder carcinoma, bronchial carcinoma, basalioma, teratoma, retinoblastoma, choroidea melanoma, seminoma, rhabdomyosarcoma, craniopharyngeoma, osteosarcoma, myosarcoma, liposarcoma, fibrosarcoma, and plasmocytoma.

Cancer may also include thyroid, breast, and lung cancer; and myeloproliferative disorders, such as polycythemia vera, thrombocythemia, myeloid metaplasia with myelofibrosis, chronic myelogenous leukemia, chronic myelomonocytic leukemia, hypereosinophilic syndrome, juvenile myelomonocytic leukemia, and systemic mast cell disease. In one embodiment, the compounds of this application are useful for treating hematopoietic disorders, in particular, acute-myelogenous leukemia (AML), chronic-myelogenous leukemia (CML), acute-promyelocytic leukemia, and acute lymphocytic leukemia (ALL).

Exemplary cancers may also include, but are not limited to, adrenocortical carcinoma, AIDS-related cancers, AIDS-related lymphoma, anal cancer, anorectal cancer, cancer of the anal canal, appendix cancer, childhood cerebellar astrocytoma, childhood cerebral astrocytoma, basal cell carcinoma, skin cancer (non-melanoma), biliary cancer, extrahepatic bile duct cancer, intrahepatic bile duct cancer, bladder cancer, urinary bladder cancer, bone and joint cancer, osteosarcoma and malignant fibrous histiocytoma, brain cancer, brain tumor, brain stem glioma, cerebellar astrocytoma, cerebral astrocytoma/malignant glioma, ependymoma, medulloblastoma, supratentorial primitive neuroectodeimal tumors, visual pathway and hypothalamic glioma, breast cancer, bronchial adenomas/carcinoids, carcinoid tumor, gastrointestinal, nervous system cancer, nervous system lymphoma, central nervous system cancer, central nervous system lymphoma, cervical cancer, childhood cancers, chronic lymphocytic leukemia, chronic myelogenous leukemia, chronic myeloproliferative disorders, colon cancer, cutaneous T-cell lymphoma, lymphoid neoplasm, mycosis fungoides, Seziary Syndrome, endometrial cancer, esophageal cancer, extracranial germ cell tumor, extragonadal germ cell tumor, extrahepatic bile duct cancer, eye cancer, intraocular melanoma, retinoblastoma, gallbladder cancer, gastric (stomach) cancer, gastrointestinal carcinoid tumor, gastrointestinal stromal tumor (GIST), germ cell tumor, ovarian germ cell tumor, gestational trophoblastic tumor glioma, head and neck cancer, hepatocellular (liver) cancer, Hodgkin lymphoma, hypopharyngeal cancer, ocular cancer, islet cell tumors (endocrine pancreas), Kaposi Sarcoma, kidney cancer, renal cancer, kidney cancer, laryngeal cancer, acute lymphoblastic leukemia, acute myeloid leukemia, chronic lymphocytic leukemia, chronic myelogenous leukemia, hairy cell leukemia, lip and oral cavity cancer, liver cancer, lung cancer, non-small cell lung cancer, small cell lung cancer, AIDS-related lymphoma, non-Hodgkin lymphoma, primary central nervous system lymphoma, Waldenstram macroglobulinemia, medulloblastoma, Merkel cell carcinoma, mesothelioma malignant, mesothelioma, metastatic squamous neck cancer, mouth cancer, cancer of the tongue, multiple endocrine neoplasia syndrome, Mycosis fungoides, myelodysplastic syndromes, myelodysplastic/myeloproliferative diseases, chronic myelogenous leukemia, acute myeloid leukemia, multiple myeloma, chronic myeloproliferative disorders, nasopharyngeal cancer, neuroblastoma, oral cancer, oral cavity cancer, oropharyngeal cancer, ovarian cancer, ovarian epithelial cancer, ovarian low malignant potential tumor, pancreatic cancer, islet cell pancreatic cancer, paranasal sinus and nasal cavity cancer, parathyroid cancer, penile cancer, pharyngeal cancer, pheochromocytoma, pineoblastoma and supratentorial primitive neuroectodermal tumors, pituitary tumor, plasma cell neoplasm/multiple myeloma, pleuropulmonary blastoma, prostate cancer, rectal cancer, renal pelvis and ureter, transitional cell cancer, retinoblastoma, rhabdomyosarcoma, salivary gland cancer, Ewing family of sarcoma tumors, Kaposi Sarcoma, soft tissue sarcoma, uterine cancer, uterine sarcoma, merkel cell skin carcinoma, small intestine cancer, soft tissue sarcoma, squamous cell carcinoma, stomach (gastric) cancer, supratentorial primitive neuroectodermal tumors, testicular cancer, throat cancer, thymoma, thymoma and thymic carcinoma, thyroid cancer, transitional cell cancer of the renal pelvis and ureter and other urinary organs, gestational trophoblastic tumor, urethral cancer, endometrial uterine cancer, uterine sarcoma, uterine corpus cancer, vaginal cancer, vulvar cancer, and Wilm's Tumor. Thus, the term ‘cancerous cell’ as provided herein, includes a cell afflicted by any one of the above-identified conditions.

A ‘cell proliferative disorder of the hematologic system’ is a cell proliferative disease or disorder involving cells of the hematologic system. A cell proliferative disorder of the hematologic system can include lymphoma, leukemia, myeloid neoplasms, mast cell neoplasms, myelodysplasia, benign monoclonal gammopathy, lymphomatoid granulomatosis, lymphomatoid papulosis, polycythemia vera, chronic myelocytic leukemia, agnogenic myeloid metaplasia, and essential thrombocythemia. A cell proliferative disorder of the hematologic system can include hyperplasia, dysplasia, and metaplasia of cells of the hematologic system. Compounds and compositions of the present application may be used to treat a cancer selected from the group consisting of a hematologic cancer or a hematologic cell proliferative disorder. A hematologic cancer can include multiple myeloma, lymphoma (including Hodgkin's lymphoma, non-Hodgkin's lymphoma, childhood lymphomas, and lymphomas of lymphocytic and cutaneous origin), leukemia (including childhood leukemia, hairy-cell leukemia, acute lymphocytic leukemia, acute myelocytic leukemia, chronic lymphocytic leukemia, chronic myelocytic leukemia, chronic myelogenous leukemia, and mast cell leukemia), myeloid neoplasms, and mast cell neoplasms.

A ‘cell proliferative disorder of the lung’ is a cell proliferative disease or disorder involving cells of the lung. Cell proliferative disorders of the lung can include all forms of cell proliferative disorders affecting lung cells. Cell proliferative disorders of the lung can include lung cancer, a precancer or precancerous condition of the lung, benign growths or lesions of the lung, and malignant growths or lesions of the lung, and metastatic lesions in tissue and organs in the body other than the lung. Compounds and compositions of the present application may be used to treat lung cancer or cell proliferative disorders of the lung. Lung cancer can include all forms of cancer of the lung. Lung cancer can include malignant lung neoplasms, carcinoma in situ, typical carcinoid tumors, and atypical carcinoid tumors. Lung cancer can include small cell lung cancer (SCLC), non-small cell lung cancer (NSCLC), squamous cell carcinoma, adenocarcinoma, small cell carcinoma, large cell carcinoma, adenosquamous cell carcinoma, and mesothelioma. Lung cancer can include ‘scar carcinoma’, bronchioalveolar carcinoma, giant cell carcinoma, spindle cell carcinoma, and large cell neuroendocrine carcinoma. Lung cancer can include lung neoplasms having histologic and ultrastructual heterogeneity (e.g., mixed cell types).

Cell proliferative disorders of the lung can also include hyperplasia, metaplasia, and dysplasia of the lung. Cell proliferative disorders of the lung can include asbestos-induced hyperplasia, squamous metaplasia, and benign reactive mesothelial metaplasia. Cell proliferative disorders of the lung can include replacement of columnar epithelium with stratified squamous epithelium, and mucosal dysplasia. Individuals exposed to inhaled injurious environmental agents such as cigarette smoke and asbestos may be at increased risk for developing cell proliferative disorders of the lung. Prior lung diseases that may predispose individuals to development of cell proliferative disorders of the lung can include chronic interstitial lung disease, necrotizing pulmonary disease, scleroderma, rheumatoid disease, sarcoidosis, interstitial pneumonitis, tuberculosis, repeated pneumonias, idiopathic pulmonary fibrosis, granulomata, asbestosis, fibrosing alveolitis, and Hodgkin's disease.

A ‘cell proliferative disorder of the colon’ is a cell proliferative disorder involving cells of the colon. A cell proliferative disorder of the colon includes colon cancer. Compounds and compositions of the present application may be used to treat colon cancer or cell proliferative disorders of the colon. Colon cancer can include all forms of cancer of the colon. Colon cancer can include sporadic and hereditary colon cancers. Colon cancer can include malignant colon neoplasms, carcinoma in situ, typical carcinoid tumors, and atypical carcinoid tumors. Colon cancer can include adenocarcinoma, squamous cell carcinoma, and adenosquamous cell carcinoma. Colon cancer can be associated with a hereditary syndrome selected from the group consisting of familial adenomatous polyposis, Gardner's syndrome, Peutz-Jeghers syndrome, Turcot's syndrome and juvenile polyposis.

Cell proliferative disorders of the colon can also include colon cancer, precancerous conditions of the colon, adenomatous polyps of the colon and metachronous lesions of the colon. A cell proliferative disorder of the colon can include adenoma. Cell proliferative disorders of the colon can be characterized by hyperplasia, metaplasia, and dysplasia of the colon. Prior colon diseases that may predispose individuals to development of cell proliferative disorders of the colon can include prior colon cancer. Current disease that may predispose individuals to development of cell proliferative disorders of the colon can include Crohn's disease and ulcerative colitis. A cell proliferative disorder of the colon can be associated with a mutation in a gene selected from the group consisting of p53, ras, FAP and DCC. An individual can have an elevated risk of developing a cell proliferative disorder of the colon due to the presence of a mutation in a gene selected from the group consisting of p53, ras, FAP and DCC.

A ‘cell proliferative disorder of the pancreas’ is a cell proliferative disorder involving cells of the pancreas. Compounds and compositions of the present application may be used to treat pancreatic cancer or cell proliferative disorders of the pancreas. Cell proliferative disorders of the pancreas can include all forms of cell proliferative disorders affecting pancreatic cells. Cell proliferative disorders of the pancreas can include pancreas cancer, a precancer or precancerous condition of the pancreas, hyperplasia of the pancreas, and dysplasia of the pancreas, benign growths or lesions of the pancreas, and malignant growths or lesions of the pancreas, and metastatic lesions in tissue and organs in the body other than the pancreas. Pancreatic cancer includes all forms of cancer of the pancreas. Pancreatic cancer can include ductal adenocarcinoma, adenosquamous carcinoma, pleomorphic giant cell carcinoma, mucinous adenocarcinoma, osteoclast-like giant cell carcinoma, mucinous cystadenocarcinoma, acinar carcinoma, unclassified large cell carcinoma, small cell carcinoma, pancreatoblastoma, papillary neoplasm, mucinous cystadenoma, papillary cystic neoplasm, and serous cystadenoma. Pancreatic cancer can also include pancreatic neoplasms having histologic and ultrastructual heterogeneity (e.g., mixed cell types).

A ‘cell proliferative disorder of the prostate’ is a cell proliferative disorder involving cells of the prostate. Compounds and compositions of the present application may be used to treat prostate cancer or cell proliferative disorders of the prostate. Cell proliferative disorders of the prostate can include all forms of cell proliferative disorders affecting prostate cells. Cell proliferative disorders of the prostate can include prostate cancer, a precancer or precancerous condition of the prostate, benign growths or lesions of the prostate, and malignant growths or lesions of the prostate, and metastatic lesions in tissue and organs in the body other than the prostate. Cell proliferative disorders of the prostate can include hyperplasia, metaplasia, and dysplasia of the prostate.

A ‘cell proliferative disorder of the skin’ is a cell proliferative disorder involving cells of the skin. Compounds and compositions of the present application may be used to treat skin cancer or cell proliferative disorders of the skin. Cell proliferative disorders of the skin can include all forms of cell proliferative disorders affecting skin cells. Cell proliferative disorders of the skin can include a precancer or precancerous condition of the skin, benign growths or lesions of the skin, melanoma, malignant melanoma and other malignant growths or lesions of the skin, and metastatic lesions in tissue and organs in the body other than the skin. Cell proliferative disorders of the skin can include hyperplasia. metaplasia, and dysplasia of the skin.

A ‘cell proliferative disorder of the ovary’ is a cell proliferative disorder involving cells of the ovary. Compounds and compositions of the present application may be used to treat ovarian cancer or cell proliferative disorders of the ovary. Cell proliferative disorders of the ovary can include all forms of cell proliferative disorders affecting cells of the ovary. Cell proliferative disorders of the ovary can include a precancer or precancerous condition of the ovary, benign growths or lesions of the ovary, ovarian cancer, malignant growths or lesions of the ovary, and metastatic lesions in tissue and organs in the body other than the ovary. Cell proliferative disorders of the skin can include hyperplasia, metaplasia, and dysplasia of cells of the ovary.

A ‘cell proliferative disorder of the breast’ is a cell proliferative disorder involving cells of the breast. Compounds and compositions of the present application may be used to treat breast cancer or cell proliferative disorders of the breast. Cell proliferative disorders of the breast can include all forms of cell proliferative disorders affecting breast cells. Cell proliferative disorders of the breast can include breast cancer, a precancer or precancerous condition of the breast, benign growths or lesions of the breast, and malignant growths or lesions of the breast, and metastatic lesions in tissue and organs in the body other than the breast. Cell proliferative disorders of the breast can include hyperplasia, metaplasia, and dysplasia of the breast.

In one embodiment, the disease or disorder includes, but is not limited to, a disease or disorders caused by or associated with Entamoeba histolytica, Pneumocystis carinii, Trypanosoma cruzi, Trypanosoma brucei, Leishmania mexicana, Clostridium histolyticum, Staphylococcus aureus, foot-and-mouth disease virus, or Crithidia fasciculata, as well as disease or disorder associated with osteoporosis, autoimmunity, schistosomiasis, malaria, tumor metastasis, metachromatic leukodystrophy, muscular dystrophy, or amyotrophy.

As modulators of a STING protein, the compounds and compositions of this application are also useful in assessing, studying, or testing biological samples. One aspect of the application relates to modulating the activity of a STING protein in a biological sample, comprising contacting the biological sample with a compound or a composition of the application.

The term ‘biological sample’, as used herein, means an in vitro or an ex vivo sample, including, without limitation, cell cultures or extracts thereof; biopsied material obtained from a mammal or extracts thereof; and blood, saliva, urine, feces, semen, tears, or other body fluids or extracts thereof. Modulation (e.g., inhibition or stimulation) of protein kinase activity in a biological sample is useful for a variety of purposes that are known to one of skill in the art. Examples of such purposes include, but are not limited to, blood transfusion, organ transplantation, and biological specimen storage.

Another aspect of this application relates to the study of a STING protein in biological and pathological phenomena; the study of intracellular signal transduction pathways mediated by STING protein. Examples of such uses include, but are not limited to, biological assays such as enzyme assays and cell-based assays.

The activity of the compounds and compositions of the present application as STING modulators may be assayed in vitro, in vivo, or in a cell line. In vitro assays include assays that determine modulation (e.g., inhibition or stimulation) of binding of a STING ligand to a STING protein through competitive binding assay. Alternate in vitro assays quantitate the ability of the agonist to bind to the protein kinase and may be measured either by radio or fluorescent labelling the agonist prior to binding, isolating the ligand/protein complex and determining the amount of radio/fluorescent label bound. Detailed conditions for assaying a compound utilized in this application as an antagonist of a STING protein are set forth in the Examples below.

In accordance with the foregoing, the present application provides a method for preventing or treating any of the diseases or disorders described above in a subject in need of such treatment, comprising administering to the subject a therapeutically effective amount of a compound of the application or an enantiomer, diastereomer, stereoisomer, or pharmaceutically acceptable salt thereof, or a pharmaceutical composition of the application. For any of the above uses, the required dosage will vary depending on the mode of administration, the particular condition to be treated and the effect desired.

Compounds and compositions of the application can be administered in therapeutically effective amounts in a combinational therapy with one or more therapeutic agents (pharmaceutical combinations) or modalities, e.g., anti-proliferative, anti-cancer, immunomodulatory, or anti-inflammatory agent, and/or non-drug therapies, etc. For example, synergistic effects can occur with anti-proliferative, anti-cancer, immunomodulatory, or anti-inflammatory substances. Where the compounds of the application are administered in conjunction with other therapies, dosages of the co-administered compounds will of course vary depending on the type of co-drug employed, on the specific drug employed, on the condition being treated and so forth.

Combination therapy may include the administration of the subject compounds in further combination with one or more other biologically active ingredients (such as, but not limited to, a second STING modulator (inhibitor or stimulator), a modulator (inhibitor or stimulator) of the cGAS-CDN-STING axis, or a modulator (inhibitor or stimulator) involved in the intracellular dsDNA mediated type-1 interferon activation. U.S. patent application Ser. No. 16/717,325 entitled MODULATING IMMUNE RESPONSES inventor Glen N. Barber, filed Dec. 17, 2019 is herein incorporated by reference in its entirety and for all purposes. Other biologically active ingredients may also include anti-proliferative agents, anti-cancer agents (e.g., chemotherapeutic agents), immunomodulatory agents, antibodies, etc. For instance, the compounds of the application can be used in combination with other pharmaceutically active compounds, preferably compounds that are able to enhance the agonist effect of the compounds of the application. The compounds of the application can be administered simultaneously (as a single preparation or separate preparation) or sequentially to the other drug therapy or treatment modality. In general, a combination therapy envisions administration of two or more drugs during a single cycle or course of therapy.

In one embodiment, the chemotherapeutic agent is an alkylating agent; an antibiotic; an anti-metabolite; a detoxifying agent; an interferon; a polyclonal or monoclonal antibody; an EGFR inhibitor; a HER2 inhibitor; a histone deacetylase inhibitor; a hormone; a mitotic inhibitor; an MTOR inhibitor; a multi-kinase inhibitor; a serine/threonine kinase inhibitor; a tyrosine kinase inhibitors; a VEGF/VEGFR inhibitor; a taxane or taxane derivative, an aromatase inhibitor, an anthracycline, a microtubule targeting drug, a topoisomerase poison drug, an inhibitor of a molecular target or enzyme (e.g., a kinase inhibitor), a cytidine analog drug, or any chemotherapeutic, anti-neoplastic or anti-proliferative agent listed in www.cancer.org/docroot/cdg/cdg_0.asp, last visited Apr. 27, 2020.

Exemplary alkylating agents include, but are not limited to, cyclophosphamide (Cytoxan; Neosar); chlorambucil (Leukeran); melphalan (Alkeran); carmustine (BiCNU); busulfan (Busulfex); lomustine (CeeNU); dacarbazine (DTIC-Dome); oxaliplatin (Eloxatin); carmustine (Gliadel); ifosfamide (Ifex); mechlorethamine (Mustargen); busulfan (Myleran); carboplatin (Paraplatin); cisplatin (CDDP; Platinol); temozolomide (Temodar); thiotepa (Thioplex); bendamustine (Treanda); or streptozocin (Zanosar).

Exemplary antibiotics include, but are not limited to, doxorubicin (Adriamycin); doxorubicin liposomal (Doxil); mitoxantrone (Novantrone); bleomycin (Blenoxane); daunorubicin (Cerubidine); daunorubicin liposomal (DaunoXome); dactinomycin (Cosmegen); epirubicin (Ellence); idarubicin (Idamycin); plicamycin (Mithracin); mitomycin (Mutamycin); pentostatin (Nipent); or valrubicin (Valstar).

Exemplary anti-metabolites include, but are not limited to, fluorouracil (Adrucil); capecitabine (Xeloda); hydroxyurea (Hydrea); mercaptopurine (Purinethol); pemetrexed (Alimta); fludarabine (Fludara); nelarabine (Arranon); cladribine (Cladribine Novaplus); clofarabine (Clolar); cytarabine (Cytosar-U); decitabine (Dacogen); cytarabine liposomal (DepoCyt); hydroxyurea (Droxia); pralatrexate (Folotyn); floxuridine (FUDR); gemcitabine (Gemzar); cladribine (Leustatin); fludarabine (Oforta); methotrexate (MTX; Rheumatrex); methotrexate (Trexall); thioguanine (Tabloid); TS-1 or cytarabine (Tarabine PFS).

Exemplary detoxifying agents include, but are not limited to, amifostine (Ethyol) or mesna (Mesnex).

Exemplary interferons include, but are not limited to, interferon alfa-2b (Intron A) or interferon alfa-2a (Roferon-A).

Exemplary polyclonal or monoclonal antibodies include, but are not limited to, trastuzumab (Herceptin); ofatumumab (Arzerra); bevacizumab (Avastin); rituximab (Rituxan); cetuximab (Erbitux); panitumumab (Vectibix); tositumomab/iodine¹³¹ tositumomab (Bexxar); alemtuzumab (Campath); ibritumomab (Zevalin; In-111; Y-90 Zevalin); gemtuzumab (Mylotarg); eculizumab (Soliris) ordenosumab.

Exemplary EGFR inhibitors include, but are not limited to, gefitinib (Iressa); lapatinib (Tykerb); cetuximab (Erbitux); erlotinib (Tarceva); panitumumab (Vectibix); PKI-166; canertinib (CI-1033); matuzumab (Emd7200) or EKB-569.

Exemplary HER2 inhibitors include, but are not limited to, trastuzumab (Herceptin); lapatinib (Tykerb) or AC-480.

Exemplary histone Deacetylase Inhibitors include, but are not limited to, vorinostat (Zolinza).

Exemplary hormones include, but are not limited to, tamoxifen (Soltamox; Nolvadex); raloxifene (Evista); megestrol (Megace); leuprolide (Lupron; Lupron Depot; Eligard; Viadur); fulvestrant (Faslodex); letrozole (Femara); triptorelin (Trelstar LA; Trelstar Depot); exemestane (Aromasin); goserelin (Zoladex); bicalutamide (Casodex); anastrozole (Arimidex); fluoxymesterone (Androxy; Halotestin); medroxyprogesterone (Provera; Depo-Provera); estramustine (Emcyt); flutamide (Eulexin); toremifene (Fareston); degarelix (Firmagon); nilutamide (Nilandron); abarelix (Plenaxis); or testolactone (Teslac).

Exemplary mitotic inhibitors include, but are not limited to, paclitaxel (Taxol; Onxol; Abraxane); docetaxel (Taxotere); vincristine (Oncovin; Vincasar PFS); vinblastine (Velban); etoposide (Toposar; Etopophos; VePesid); teniposide (Vumon); ixabepilone (Ixempra); nocodazole; epothilone; vinorelbine (Navelbine); camptothecin (CPT); irinotecan (Camptosar); topotecan (Hycamtin); amsacrine or lamellarin D (LAM-D).

Exemplary MTOR inhibitors include, but are not limited to, everolimus (Afinitor) or temsirolimus (Torisel); rapamune, ridaforolimus; or AP23573.

Exemplary multi-kinase inhibitors include, but are not limited to, sorafenib (Nexavar); sunitinib (Sutent); BIBW 2992; E7080; Zd6474; PKC-412; motesanib; or AP24534.

Exemplary serine/threonine kinase inhibitors include, but are not limited to, ruboxistaurin; eril/easudil hydrochloride; flavopiridol; seliciclib (CYC202; Roscovitrine); SNS-032 (BMS-387032); Pkc412; bryostatin; KAI-9803;SF1126; VX-680; Azd1152; Arry-142886 (AZD-6244); SCIO-469; GW681323; CC-401; CEP-1347 or PD 332991.

Exemplary tyrosine kinase inhibitors include, but are not limited to, erlotinib (Tarceva); gefitinib (Iressa); imatinib (Gleevec); sorafenib (Nexavar); sunitinib (Sutent); trastuzumab (Herceptin); bevacizumab (Avastin); rituximab (Rituxan); lapatinib (Tykerb); cetuximab (Erbitux); panitumumab (Vectibix); everolimus (Afinitor); alemtuzumab (Campath); gemtuzumab (Mylotarg); temsirolimus (Torisel); pazopanib (Votrient); dasatinib (Sprycel); nilotinib (Tasigna); vatalanib (Ptk787; ZK222584); CEP-701; SU5614; MLN518; XL999; VX-322; Azd0530; BMS-354825; SKI-606 CP-690; AG-490; WHI-P154; WHI-P131; AC-220; or AMG888.

Exemplary VEGF/VEGFR inhibitors include, but are not limited to, bevacizumab (Avastin); sorafenib (Nexavar); sunitinib (Sutent); ranibizumab; pegaptanib; or vandetinib. Exemplary microtubule targeting drugs include, but are not limited to, paclitaxel, docetaxel, vincristin, vinblastin, nocodazole, epothilones and navelbine.

Exemplary topoisomerase poison drugs include, but are not limited to, teniposide, etoposide, adriamycin, camptothecin, daunorubicin, dactinomycin, mitoxantrone, amsacrine, epirubicin and idarubicin.

Exemplary taxanes or taxane derivatives include, but are not limited to, paclitaxel and docetaxol.

Exemplary general chemotherapeutic, anti-neoplastic, anti-proliferative agents include, but are not limited to, altretamine (Hexalen); isotretinoin (Accutane; Amnesteem; Claravis; Sotret); tretinoin (Vesanoid); azacitidine (Vidaza); bortezomib (Velcade) asparaginase (Elspar); levamisole (Ergamisol); mitotane (Lysodren); procarbazine (Matulane); pegaspargase (Oncaspar); denileukin diftitox (Ontak); porfimer (Photofrin); aldesleukin (Proleukin); lenalidomide (Revlimid); bexarotene (Targretin); thalidomide (Thalomid); temsirolimus (Torisel); arsenic trioxide (Trisenox); verteporfin (Visudyne); mimosine (Leucenol); (1M tegafur-0.4 M 5-chloro-2,4-dihydroxypyrimidine-1 M potassium oxonate) or lovastatin.

Exemplary kinase inhibitors include, but are not limited to, Bevacizumab (targets VEGF), BIBW 2992 (targets EGFR and Erb2), Cetuximab/Erbitux (targets Erb1), Imatinib/Gleevic (targets Bcr-Abl), Trastuzumab (targets Erb2), Gefitinib/Iressa (targets EGFR), Ranibizumab (targets VEGF), Pegaptanib (targets VEGF), Erlotinib/Tarceva (targets Erb1), Nilotinib (targets Bcr-Abl), Lapatinib (targets Erb1 and Erb2/Her2), GW-572016/lapatinib ditosylate (targets HER2/Erb2), Panitumumab/Vectibix (targets EGFR), Vandetinib (targets RET/VEGFR), E7080 (multiple targets including RET and VEGFR), Herceptin (targets HER2/Erb2), PKI-166 (targets EGFR), Canertinib/CI-1033 (targets EGFR), Sunitinib/SU-11464/Sutent (targets EGFR and FLT3), Matuzumab/Emd7200 (targets EGFR), EKB-569 (targets EGFR), Zd6474 (targets EGFR and VEGFR), PKC-412 (targets VEGR and FLT3), Vatalanib/Ptk787/ZK222584 (targets VEGR), CEP-701 (targets FLT3), SU5614 (targets FLT3), MLN518 (targets FLT3), XL999 (targets FLT3), VX-322 (targets FLT3), Azd0530 (targets SRC), BMS-354825 (targets SRC), SKI-606 (targets SRC), CP-690 (targets JAK), AG-490 (targets JAK), WHI-P154 (targets JAK), WHI-P131 (targets JAK), sorafenib/Nexavar (targets RAF kinase, VEGFR-1, VEGFR-2, VEGFR-3, PDGFR-β, KIT, FLT-3, and RET), Dasatinib/Sprycel (BCR/ABL and Src), AC-220 (targets Flt3), AC-480 (targets all HER proteins, ‘panHER’), Motesanib diphosphate (targets VEGF1-3, PDGFR, and c-kit), Denosumab (targets RANKL, inhibits SRC), AMG888 (targets HER3), and AP24534 (multiple targets including Flt3).

In one embodiment, the compounds may be administered in combination with one or more separate pharmaceutical agents, e.g., a chemotherapeutic agent, an immunotherapeutic agent, or an adjunctive therapeutic agent.

As used herein, ‘combination therapy’ or ‘co-therapy’ includes the administration of a compound of the present application, or a pharmaceutically acceptable salt or ester thereof, and at least a second agent as part of a specific treatment regimen intended to provide the beneficial effect from the co-action of these therapeutic agents. The beneficial effect of the combination includes, but is not limited to, pharmacokinetic or pharmacodynamic co-action resulting from the combination of therapeutic agents. Administration of these therapeutic agents in combination typically is carried out over a defined time period (usually minutes, hours, days or weeks depending upon the combination selected). ‘Combination therapy’ may be, but generally is not, intended to encompass the administration of two or more of these therapeutic agents as part of separate monotherapy regimens that incidentally and arbitrarily result in the combinations of the present application.

‘Combination therapy’ is intended to embrace administration of these therapeutic agents in a sequential manner, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single capsule having a fixed ratio of each therapeutic agent or in multiple. single capsules for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection. The sequence in which the therapeutic agents are administered is not narrowly critical.

‘Combination therapy’ also embraces the administration of the therapeutic agents as described above in further combination with other biologically active ingredients and non-drug therapies (e.g., surgery or radiation treatment). Where the combination therapy further comprises a non-drug treatment, the non-drug treatment may be conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, perhaps by days or even weeks.

The elimination of apoptotic cells by phagocytes generally transpires without triggering deleterious inflammatory events that could be harmful to the host. This process has evolved to trigger the rapid degradation of self-nucleic acid within the dying cell by host DNAses. Failure to degrade genomic DNA from such cells can lead to the inadvertent stimulation of DNA-activated innate immune sensors in engulfing phagocytes, such as those controlled by STING signaling, with lethal pro-inflammatory consequences. Cancer cells are notoriously non-immunogenic in part, since they are phagocytosed comparable to normal apoptotic cells and avoid transiently triggering innate immune pathways required to facilitate tumor cell antigen presentation and the cross-priming of T-cells. It is therefore unclear how antigen presenting cells (APCs) immunologically recognize a DNA-damaged, potentially pre-cancerous cell, to facilitate anti-tumor CTL activity and immune clearance. Generally, the cytosol of the cell is free of dsDNA species, unless introduced following microbial infection or leaked from the nucleus after cellular division or following DNA-damage. Cytosolic dsDNA species associate with the synthase cGAS. to generate cyclic dinucleotides (CDN's) that bind to and trigger intrinsic STING signaling presumably to activate host defense countermeasures, including attracting phagocytes to the microenvironment. However, the exact mechanisms, including the identification of key inducible genes important for this process remain to be identified. Subsequently, efficient APC activation requires extrinsic cGAS/STING-dependent signaling, a process that can also be triggered in trans, by dsDNA species present in the engulfed cell that has escaped complete DNase degradation.

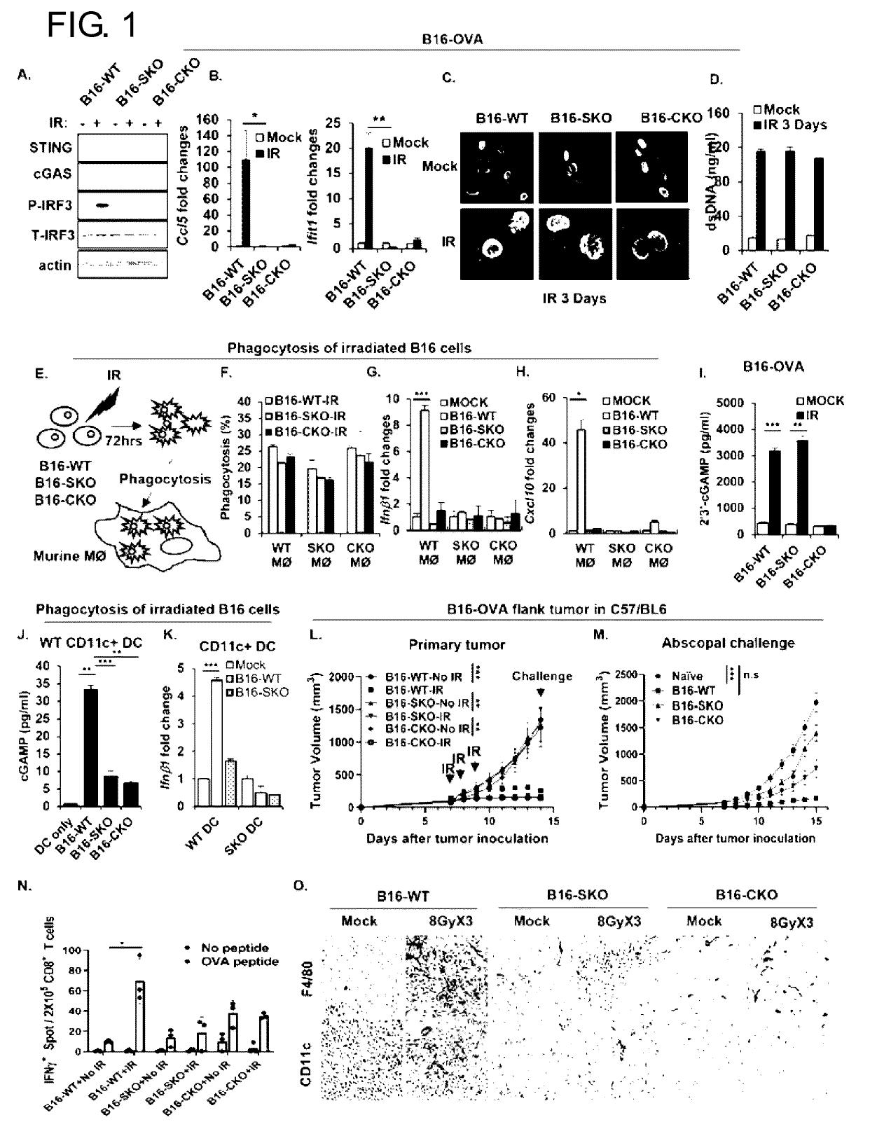

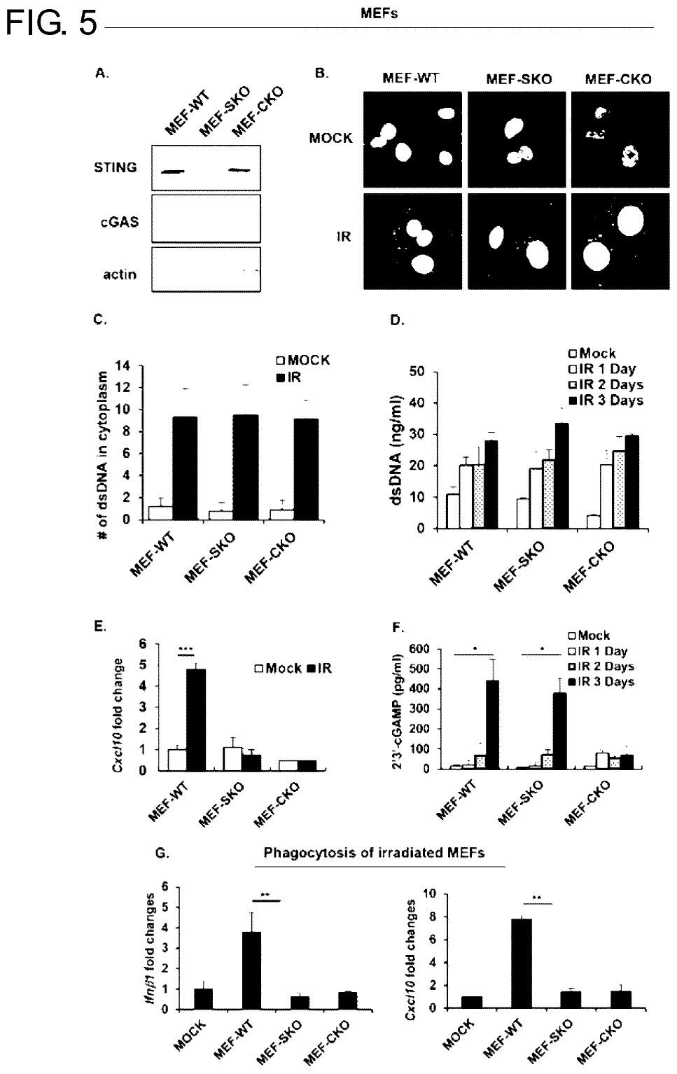

To further examine the mechanisms of APC activation and importance of STING signaling in this process, a B16 murine melanoma model (expressing OVA) which is syngeneic for immunocompetent C57/BL6 mice was utilized. B16-OVA cells have a relatively intact cytosolic DNA signaling since they express both cGAS and STING. Accordingly, B16-OVA cells lacking expression of STING or cGAS as controls were also generated. DNA-damage, instigated by chemotherapy or irradiation, causes chromatin instability and the generation of cytoplasmic micronuclei (CM) containing self-dsDNA species, such as cytoplasmic chromatin fragments (CCF), capable of activating intrinsic cytokine production in a STING-dependent manner. Loss of STING signaling prevented cytokine production following irradiation, such as production of Ccl5, although treated B16 cells generated similar amounts of cytosolic DNA species, regardless of the presence or absence of STING activity (FIGS. 1A-D and FIGS. 5A-D). An in vitro assay to monitor the activation of phagocytes by irradiated cells, in trans (FIG. 1E) was developed. Irradiated B16 cells containing, or lacking STING appeared to be phagocytosed efficiently and equally, despite the presence or absence of STING or cGAS (FIG. 1F). It was confirmed that irradiated B16-OVA cells were able to activate phagocytes as measured by type I IFN or CXCL10 production, in trans, which was dependent on intact intrinsic STING signaling in the irradiated cells (FIGS. 1G, H). This event was also reliant on extrinsic STING signaling in the phagocyte, indicating that cGAS/STING agonists such as CDNs or dsDNA species in B16-OVA cells were required for this trans activating activity (FIG. 1G, H). However, since irradiated B16-OVA cells lacking STING, though containing CDN's, remained unable to activate phagocytes, the observations suggest involvement of dsDNA species, rather than any bystander effects caused by intrinsically generated trans acting cGAMP (FIG. 1I). The importance of dsDNA rather than bystander CDN's in phagocytic activation was supported by noting that irradiated cells lacking STING though containing CDN's (B16-SKO), failed to activate phagocytes in trans, as measured by extrinsic cGAMP production and type I IFN production (FIGS. 1J, 1K). The data suggests that STING signaling as well as cytosolic dsDNA species in irradiated, dying cells are both requisite for the activation of APC's in trans and does not predominantly require bystander cGAMP activity. The data indicating the importance of intrinsic STING signaling and STING-induced genes in facilitating the trans activation of phagocytosis was verified using normal murine embryonic fibroblasts (MEFs) that lacked STING or cGAS (FIGS. 5E-G).

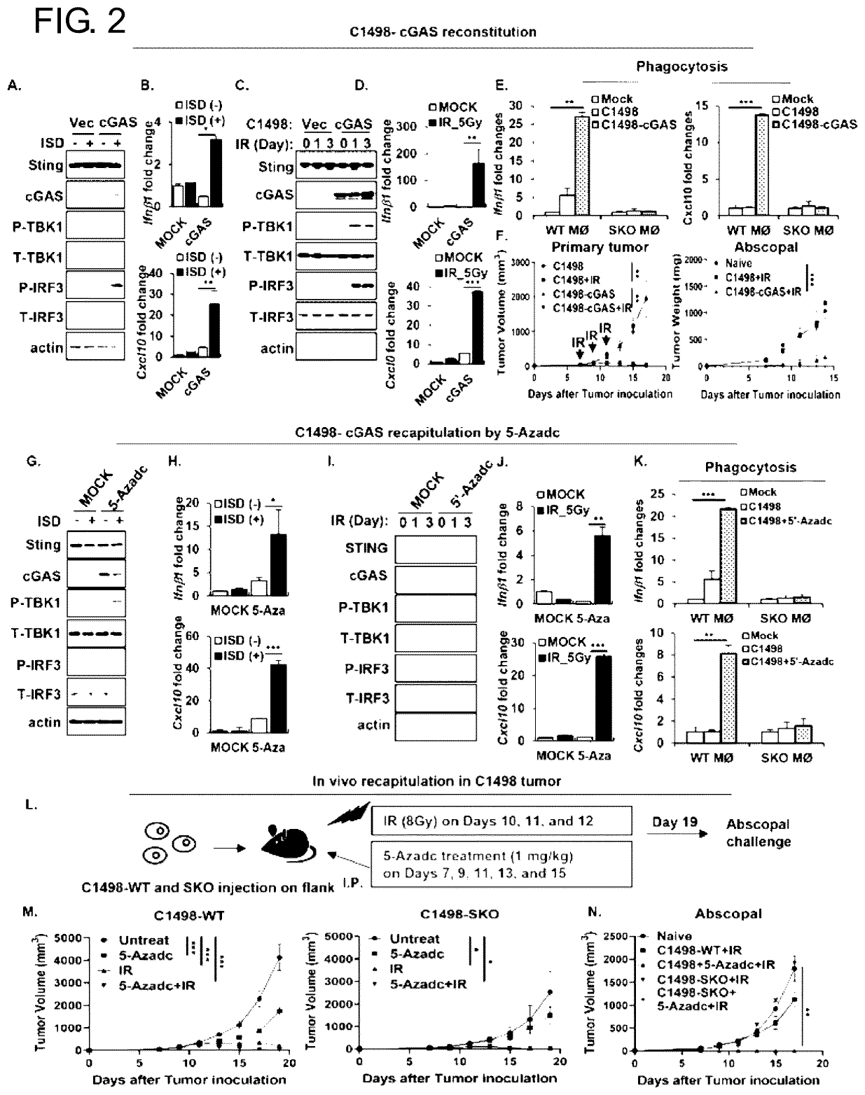

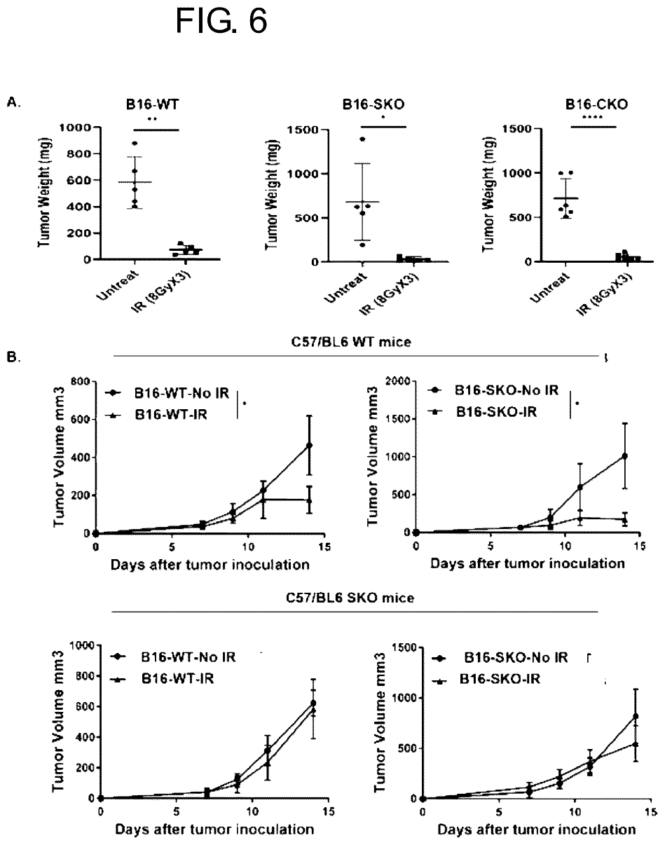

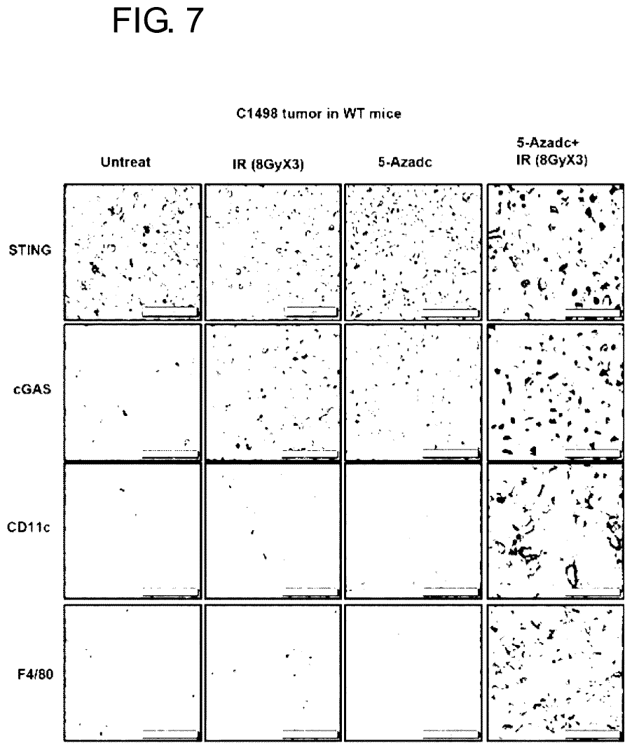

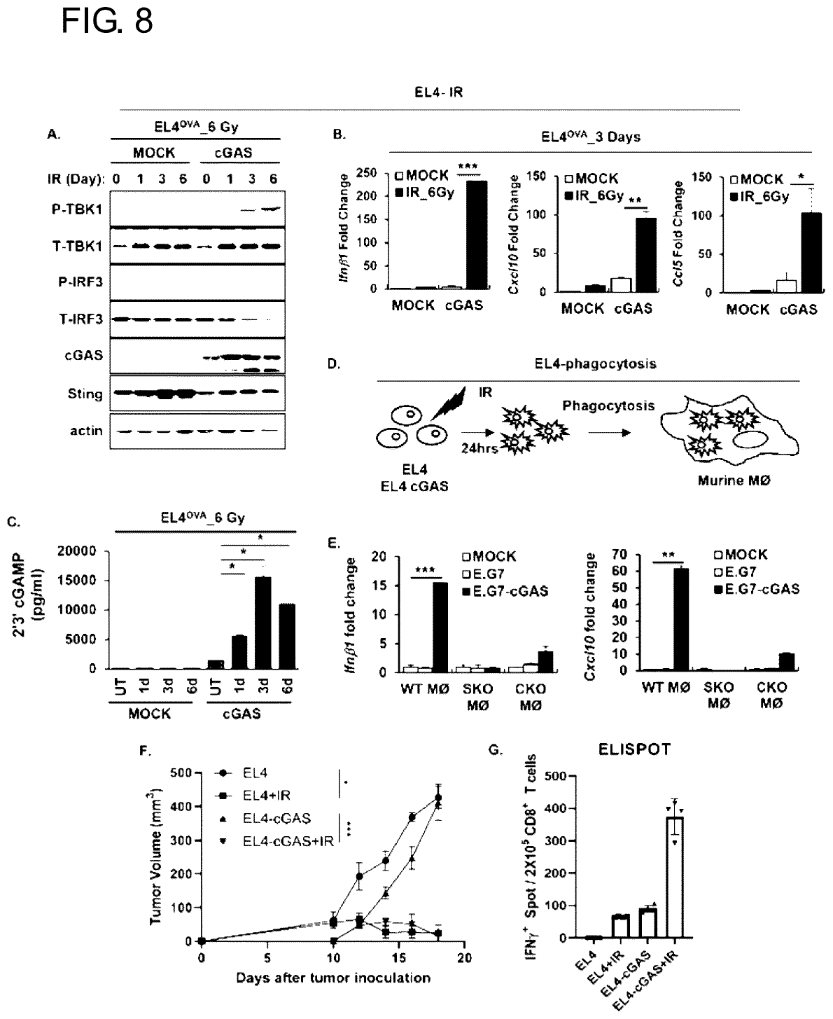

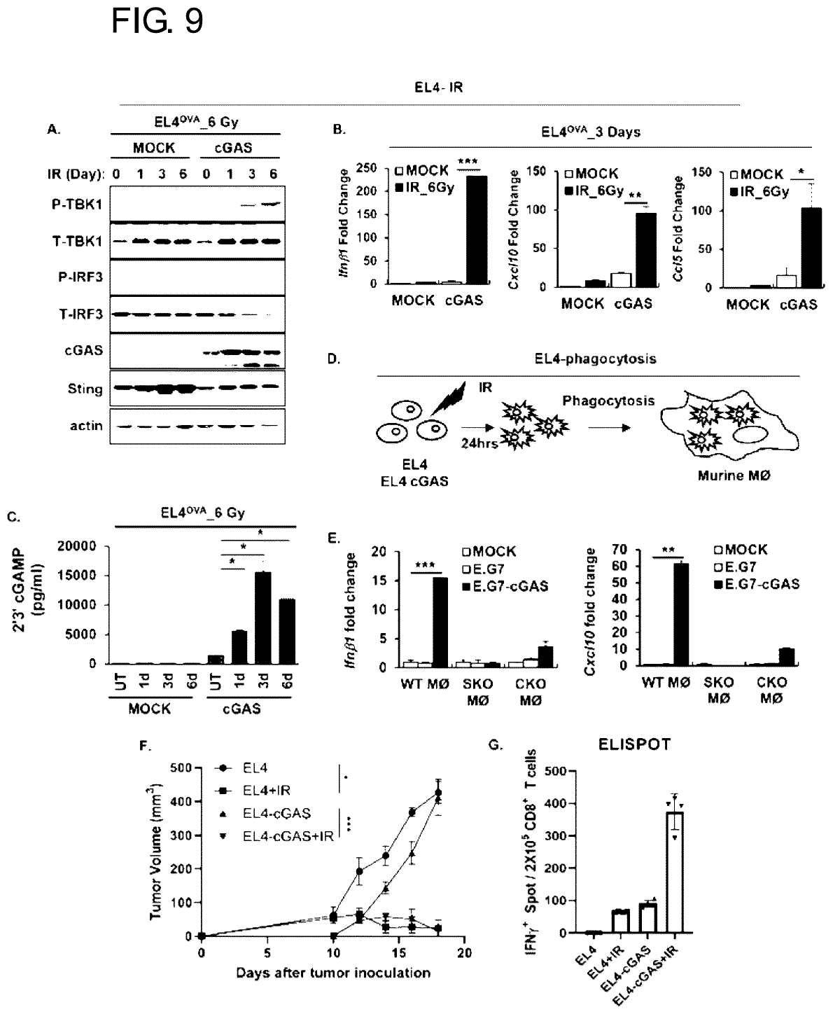

To evaluate the significance of these observations in vivo, B16-OVA containing or lacking STING or cGAS were orthotopically grown on the flanks of syngeneic C57/BL mice. It was noted that irradiation efficiently eliminated the growth of the tumors, causing cellular death (FIG. 1L, xxx FIG. 6A). However, intrinsic STING signaling in the tumor cell was required to generate efficient anti-OVA T cell activity and subsequently prevent abscopal tumor growth an effect that similarly did not involve gGAMP bystander activity, since B16-SKO cells, harboring CDN's, were largely unable to achieve this effect (FIGS. 1M and 1N). Accordingly, after irradiation, a high infiltration of macrophages and dendritic cell subsets were observed in the tumor microenvironment (TME) which was also dependent intact STING signaling in the irradiated tumor cells (FIG. 1O). This activity was also confirmed as being dependent on extrinsic STING signaling since STING-deficient mice, harboring orthotopic B16-WT tumors were unable to mount the observed anti-tumor activity following irradiation therapy (FIG. 6B). A variety of evidence now indicates that STING signaling is suppressed in tumor cells, for reasons that remain to be fully clarified. However, the above data suggests that loss of STING would enable DNA-damaged cells to escape the activation of phagocytes (FIGS. 1E-H). To extend these findings, the immunological consequences of reconstituting STING signaling in tumor cells lacking STING or cGAS expression was examined. For this approach, C1498 cells, a murine model (C57/BL6) for acute myelogenous leukemia (AML) that it was noted exhibits defective STING signaling through loss of cGAS expression (FIG. 2A) were used. C1498 tumor cells were stably reconstituted with cGAS. Following transfection with dsDNA species, or following irradiation, it was noted that cytosolic dsDNA-dependent cytokine expression was rescued only in the cGAS expressing cells (FIGS. 2A-D). Further, C1498 tumors containing reconstituted cGAS (STING signaling) were able to activate of APC's, in trans following irradiation (FIG. 2E). Subsequently, C1498 cells lacking or expressing cGAS were implanted onto the flanks of C57/BL6 mice and irradiated. The effects of radiation treatment greatly reduced growth of all tumors, but only the irradiated C1498 cells expressing cGAS resulted in the abscopal effect of inhibiting C1498 tumors on the opposite flanks of the mice (FIG. 2F). These results indicate that reconstituting intrinsic STING signaling in vivo can rescue anti-tumor immunity. Loss of STING or cGAS expression in tumor cells typically occurs through epigenetic silencing, an event that can sometimes be reversed using anti-methylation compounds such as 5-Azacytidine (5-Azadc). Indeed, 5-Azadc treatment was found to rescue cGAS expression in C1498 cells and similarly rendered such treated cells responsive to dsDNA transfection or irradiation as evidence by the phosphorylation of TBK1 and IRF3 dependent cytokine production (FIG. 2G-J). Furthermore, only 5-Azade treated C1498 irradiated cells potently activated APC's, following phagocytosis, in trans (FIG. 2K). This event was similarly dependent on extrinsic STING signaling in host macrophages (FIG. 2K). C1498 tumors were implanted into the flanks of C57/BL6 mice and treated the mice, I.P., with 5-Azadc, prior to tumor irradiation. Tumor growth suppression following radiation treatment was observed, but again only irradiated C1498 cells containing a cGAS rescued STING signaling pathway prevented the abscopal growth of similar tumors on the opposite flanks of the mice (FIG. 2L-N). Immunohistochemical analysis confirmed the expression of cGAS only in the 5-Azadc treated mice as well as significant infiltration of CD11c expressing hematopoietic cells within the tumor microenvironment as well as the generation of anti-tumor CTLs (FIG. 7). Similar results were observed using yet another model tumor, EL4, a T-cell lymphoblastic lymphoma (ALL) that similarly lacks cGAS expression. Reconstitution of cGAS and thus STING signaling rendered EL4, which also expressed OVA, sensitive to radiation induced cytokine production and the activation of phagocytes in trans leading to an increase in anti-OVA CTL activity, in vivo (FIG. 8). To extend these studies to human cancer cells, a melanoma cell-line A375 that lacked STING signaling through comparable loss of cGAS was examined. Correspondingly, reconstitution of cGAS by stable transfection or through 5-Azadc treatment, rescued the ability of such cells to produce cytokines in response to radiation treatment and rendered them able to potently activate APC's in trans as determined by the production of type I IFN and CXCL10 (FIG. 9). The data indicates that intrinsic STING signaling caused by DNA damaged likely stimulates the transcription of inducible genes essential for the efficient immunogenic trans activation of phagocytosing APC's. Further, reconstituting STING signaling, and presumably, key inducible genes, in vivo, using established demethylating agents may augment the immunomodulatory effects of radiation therapy. Conversely, resistance to the immunogenic effects of radiation may be at least partially explained by defective STING-signaling in tumor cells.

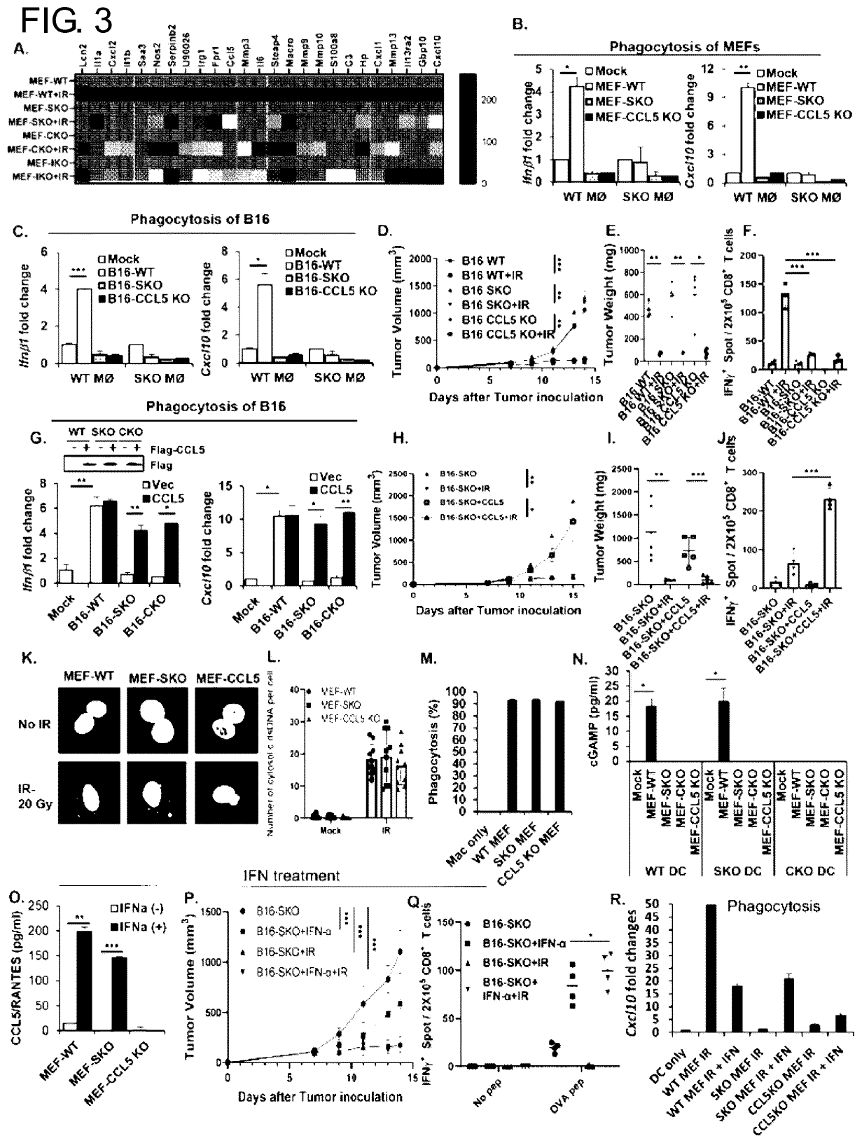

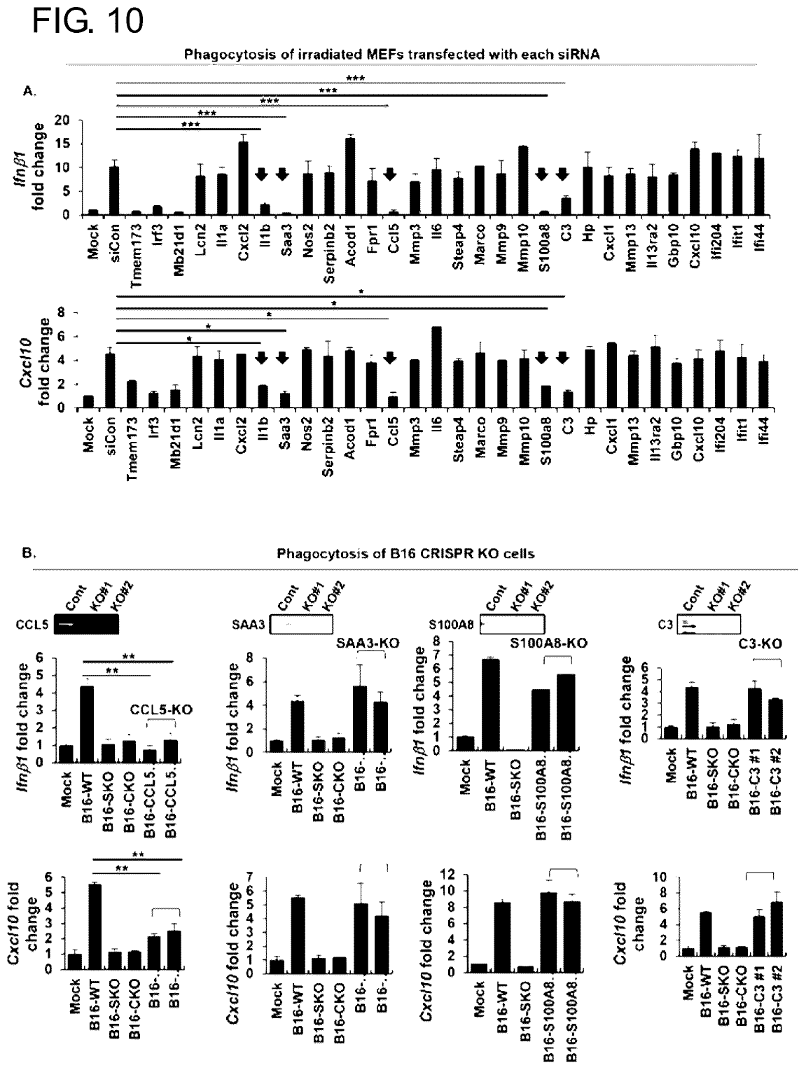

To identify the STING-dependent genes that play a role in facilitating the immunogenicity of DNA-damaged cells, normal MEFs that lacked STING, cGAS or IRF3 were irradiated. Transcription profile analysis indicated the stimulation of a number of STING-induced genes in response to radiation (FIG. 3A). To identify STING-induced genes that plausibly influence the transactivation of phagocytes, wild-type MEFs were treated with RNAi, targeting the top STING-controlled, radiation induced genes. RNAi-treated cells were then irradiated and incubated in vitro with phagocytes to ascertain if any of the silenced genes influenced trans activation. This approach predominantly indicated that loss of STING-inducible Ccl5, in irradiated MEFs significantly prevented the in vitro activation of phagocytes, in trans (FIG. 3B, FIG. 10A). The observations using RNAi as well as CRISPR technology confirmed that loss of intrinsic Ccl5 expression in B16 cells (B16-CCL5 KO) inhibited the ability of irradiated cells to active phagocytes, in trans (FIG. 3C; FIG. 10B). This event was also dependent on extrinsic cGAS signaling within the phagocytes themselves, confirming a key role for dsDNA in the activation process (FIG. 3B, C). To evaluate the importance of Ccl5, in vivo, B16 cells lacking STING or Ccl5 were implanted into C57/BL6 mice and when palpable, irradiated. While irradiation suppressed tumor growth via direct cell damage, loss of Ccl5 suppressed bystander immunogenic effects, similar to loss of STING and prevented the efficient generation of CTL's (FIGS. 3D-F). Since loss of STING signaling in cells renders them significantly non-immunogenic in response to DNA-damage, it was conceivable that reconstitution of Ccl5 may rescue immunogenicity following irradiation. B16 cells lacking STING or cGAS were thus transfected with Ccl5 and irradiated prior to being co-cultured with phagocytes. This data indicated that reconstitution of Ccl5 expression in B16 SKO or CKO cells significantly increased their ability to stimulate phagocytes following irradiation (FIG. 3G). In addition, B16 cells lacking STING (B16-SKO) and reconstituted with Ccl5, regained ability to stimulate CTL's, in vivo, following radiation treatment (FIG. 3H-J). The data indicates that Ccl5 appears to be a key STING-inducible, IRF3 induced gene required to render DNA-damaged cells competent to stimulate phagocytes in trans.

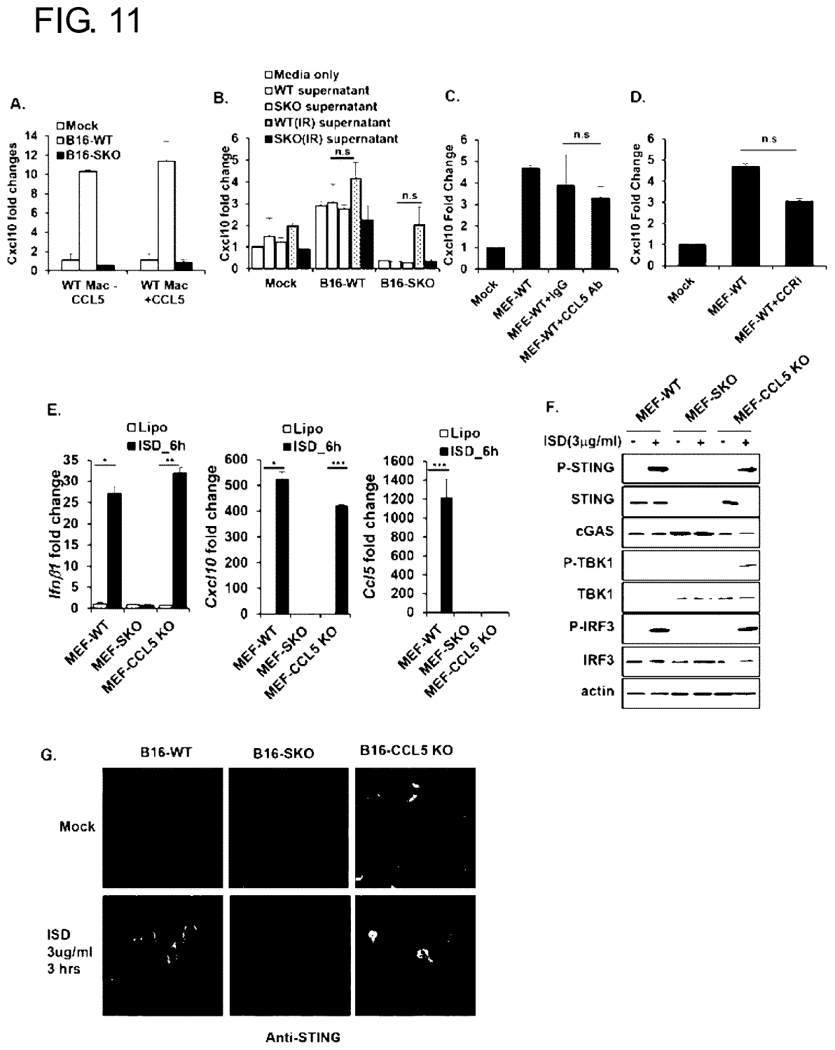

Secreted Ccl5 exerted its effects through a paracrine or autocrine fashion to influence the immunogenicity of DNA damaged cells and/or the activation of phagocytes. However, exogenously added Ccl5 did not appear to significantly rescue the immunogenicity of Ccl5 KO cells or STING KO cells nor influence the activation of phagocytes, suggesting that Ccl5 exerted its effects through alternate mechanisms (FIG. 11A). This was corroborated by demonstrating that the media bathing irradiated WT B16, SKO or Ccl5 MEFs cells did not strongly influence the activation of APC's, again suggesting that cell-to cell phagocytic contact was important (FIG. 11B). In addition, blocking CCL5 by anti-CCL5 or blocking CCL5 receptors using pan-CCR inhibitors did not affect cytokine expression in WT phagocytes engulfing irradiated cells (FIGS. 11C and 11D). Neither did loss of Ccl5 influence the generation of cytoplasmic micronuclei in irradiated cells (FIG. 3K and L), or influence the ability of phagocytes to engulf such cells (FIG. 3M). Ccl5 deficiency did not influence STING function, such as trafficking or the activation of alternate STING-inducible genes (FIGS. 11E-G). The generation of cGAMP within phagocytes was only observed following the incubation with irradiated WT MEFs and was not observed with irradiated SKO, CKO or CCL5 KO MEFs (FIG. 3N). The data suggests that loss of STING-inducible Ccl5 does not affect the generation of cytoplasmic nuclei, nor the phagocytosis of DNA-damaged cells ('find me cat me signals') but rather influences the ability of cytosolic DNA within irradiated cells to efficiently trans-activate phagocytes, via cGAS activation.

It is evident that some STING-inducible, IRF3-dependent genes including Ccl5 are known to be activated by type I interferon (IFN) through JAK/STAT signaling and the activation of IRF7 7-9. It was evaluated whether tumor cells lacking STING immunogenicity could be rendered immunogenic by augmenting Ccl5 production using type I IFN. This approach confirmed the induction of Ccl5 in B16-SKO cells by type I IFN (IFNa) (FIG. 30). Next. B16 tumor cells lacking STING and expressing OVA (B16-OVA-SKO) were implanted into C57/BL6 mice and after they became palpable, the mice were inoculated i.p. with type I IFN, before irradiating the tumors. It was confirmed that radiation treatment readily suppressed the growth of B16-OVA-SKO tumors but did not generate robust anti-OVA CTL activity (FIGS. 1P and 1Q). The addition of type I IFN alone, while marginally affecting overall tumor growth was observed to increase CTL activity to OVA (FIGS. 1P and 1Q). However, the combination of radiation treatment in the presence of type I IFN greatly reduced tumor growth while rescuing the ability to generate CTL activity (FIGS. 1P and 1Q). The importance of Ccl5 was again highlighted by demonstrating that irradiated CCL5-deficient MEFs exhibited diminished ability to activate APC's even when treated with type I IFN (FIG. 3R). The data suggests that the presence of type I IFN may partially compensate for loss of STING signaling by inducing overlapping inducible genes such as Ccl5 and may facilitate the effectiveness of DNA damaging anti-cancer treatments, including radiation.

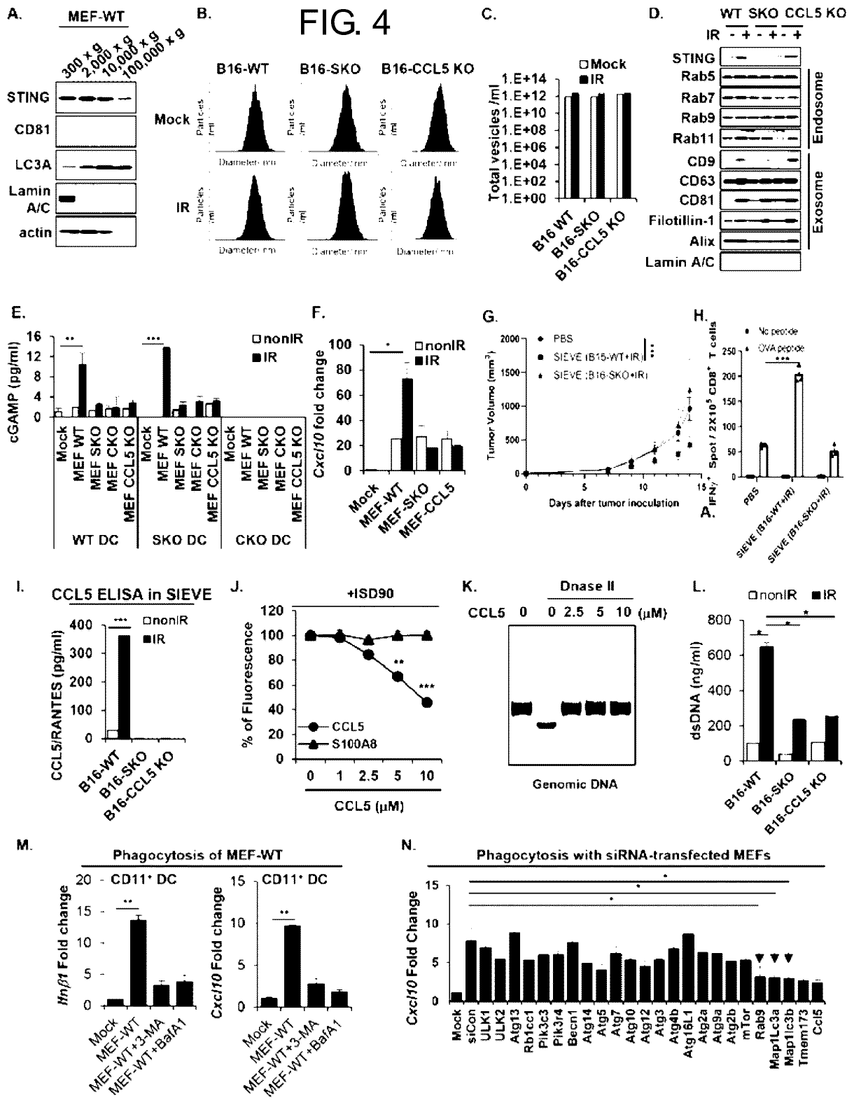

The data indicates that cytosolic DNA-species within DNA-damaged cells can potently activate phagocytes, an event dependent on STING-inducible genes such as Ccl5. It is thus plausible that CM. CCF's or other intrinsic endosomal vesicles harboring STING-induced proteins may themselves play a role in these observations. To address this cells expressing or lacking STING or Ccl5 were irradiated and centrifugation to isolate intracellular endosomes/autophagosomes was performed to determine what could plausibly play a role in phagocytic activation. Low speed centrifugation of irradiated MEFs removed cytoplasmic micronuclei, as confirmed by the presence of Lamin A/C (FIG. 4A). High speed centrifugation yielded similar quantities of intracellular vesicles containing or lacking STING or Ccl5, that measured approximately 100-120 nm diameters in length, as determined by nanoparticle tracking analysis (FIGS. 4B, 4C). These likely consisted of a collection of dynamic trafficking endosome/autophagosomes which were Lamin A/C negative but microtubule-associated protein 1A/1B-light chain 3 (LC3) positive together with both endosomal and pre-exosomal markers such as Rab9, GTPase and CD81 (FIG. 4D). STING, which traffics via non-canonical autophagy to facilitate the phosphorylation of IRF3 by TBK1 and which has also been reported to associate with and facilitate LC3 lipidation, was similarly detected in these fractions (FIG. 4D). Vesicles from irradiated MEF cells containing or lacking STING or Ccl5 were then examined for their ability to stimulate cGAS in murine DC's, containing or lacking cGAS or STING by measuring cGAMP production. This analysis indicated that only vesicles isolated from WT cells retained the ability to trigger cGAMP production in the treated DC's, again suggesting the importance of dsDNA species in these observations (FIG. 4E). Accordingly, vesicles from irradiated WT cells, but not cells lacking cGAS or STING, were also capable of stimulating STING-dependent Cxcl10 production in treated phagocytes (FIG. 4F). To further evaluate the immunogenic nature of the STING-inducible endosomal vesicles (SIEVE's), B16-OVA cells were inoculated into the flanks of C57/BL6 mice. SIEVE's isolated from irradiated B16-OVA cells of B16-OVA cells lacking STING were injected into palpable tumors. It was observed that tumors grew significantly slower following intratumoral (I.T.) treatment using SIEVE's isolated from WT B16-OVA cells but not B1-OVA-SKO cells (FIG. 4G). The slowing of tumor growth was concomitant with the generation of anti-OVA CTL's only in SIEVE-immunized mice (FIG. 4H).

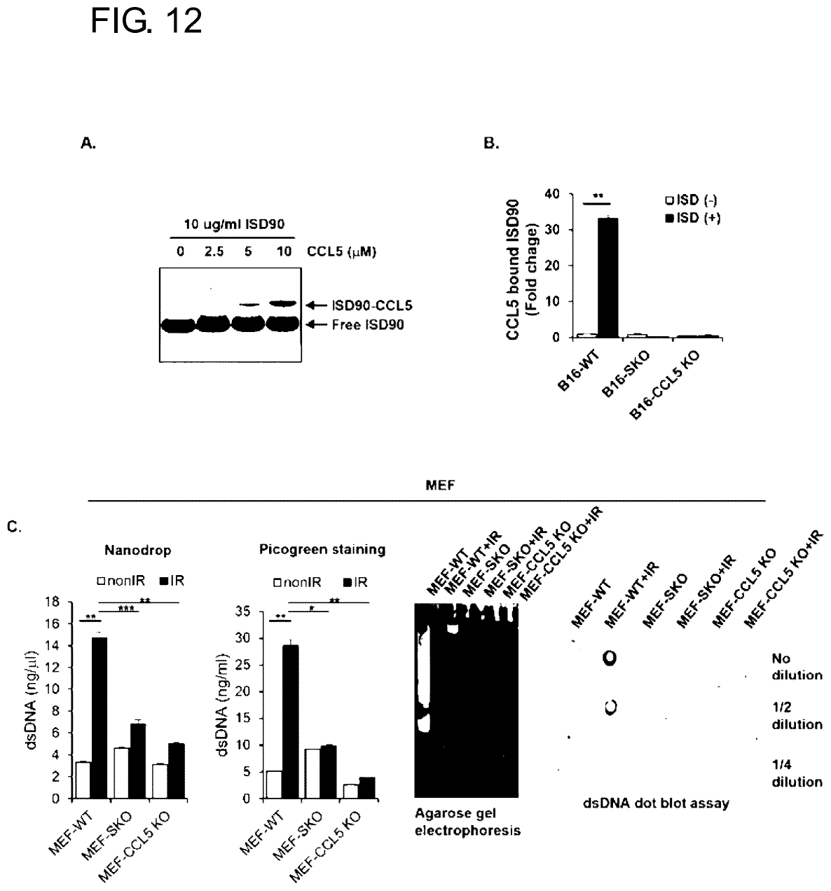

Chemokines such as CXCL4 have been demonstrated as associating with DNA to form stable nanoparticle complexes capable of robustly activating members of the Toll-like receptor (TLR). In a similar fashion, it was proposed that Ccl5 facilitates the association of dsDNA species into SIEVE-like vesicles and/or protects dsDNA from intracellular degradation processes. Indeed, the presence of Ccl5 within SIEVEs was confirmed and it was observed that Ccl5 associated with and conferred protection against DNA degradation following exposure to the key phagocytic nuclease DNaseII (FIGS. 4I-L). A reduction in the presence of dsDNA within vesicles isolated from STING or Ccl5-deficient cells, was perceptible compared to WT cells (FIG. 12). Ablation of DNasell leads to chronic cytokine stimulation in phagocytes via STING signaling since such cells are unable to digest genomic DNA from engulfed apoptotic bodies. SIEVEs may evade degradation within phagocytic lysosomal compartments after engulfment, to retain ability to activate the extrinsic STING pathway.

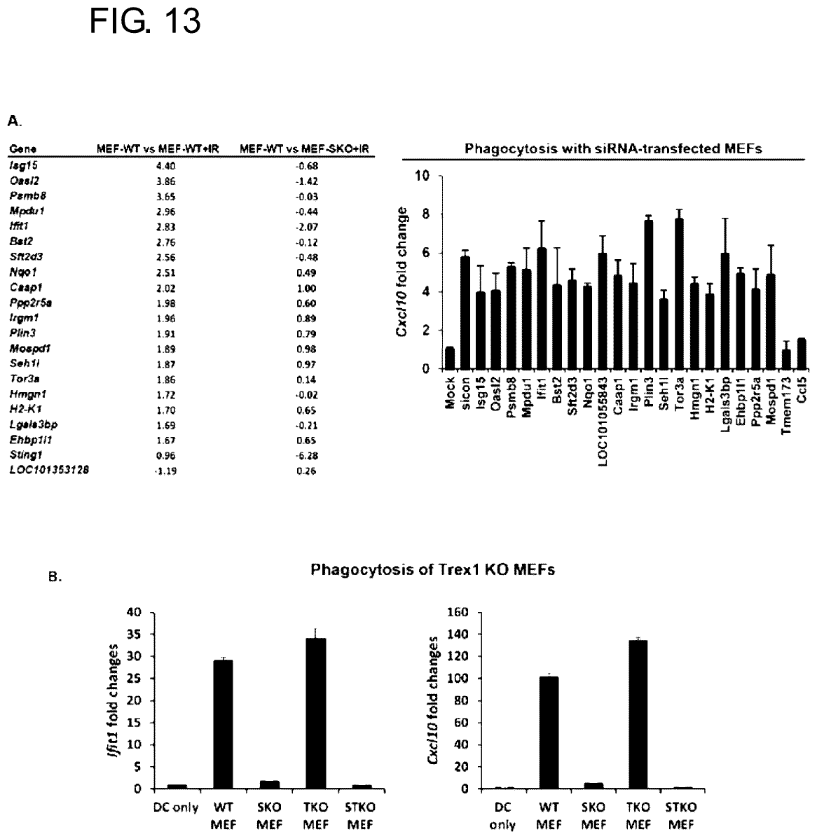

The data indicates that immunogenic SEIVE's, likely exist as a subset within the dynamic interconnecting endosome/autophagosome family and may contain STING-inducible proteins including ISG15 and IFIT1, as suggested by additional proteomic analysis (FIG. 13A). However, knockdown of many of these genes, or the DNase TREX1 did not appreciably affect the immunogenicity of irradiated cells, unlike loss of STING itself or Ccl5, indicating the specificity of the observations (FIG. 13B). Since the formation of SIEVEs indicated involvement with the autophagy machinery, as also confirmed by immunoblot and proteomic analysis, it was examined whether the autophagy inhibitors 3-Methyladenine (3-MA) bafilomycin A1 (BafA1) influenced the ability of irradiated MEF cells to activate APC's (FIG. 4D). This data confirmed that 3-MA and BafA1 suppressed the immunogenicity of irradiated MEF cells (FIG. 4M). Key endosomal/autophagy related proteins in MEFs were further knocked down, using an autophagy genes RNAi library, prior to irradiation treatment and incubation with phagocytes. This data implicated that suppression of microtubule-associated protein 1A/1B-light chain 3 (LC3), Map1Lc3a and Map1Lc3b as well as Ras-related protein Rab-9B, but not other autophagy-associated molecules, predominantly repressed the immunogenicity of irradiated cells (FIG. 4N). Collectively, the data suggests that STING activation leads to the generation of host defense proteins that facilitate the formation of immunogenic, dsDNA-containing endosomic vesicles capable of stimulating extrinsic cGAS/STING signaling in phagocytes.

The study indicates that defective STING signaling renders DNA-damaged cells non-immunogenic through loss of cytosolic-DNA stimulated STING-inducible genes and identifies intrinsic Ccl5 as a key player in this process. Loss of STING signaling may be an early prerequisite in the transformation process that enables DNA-damaged cells to avoid not only the generation of cytokines which may attract the immune surveillance system, but also to avoid the direct activation of APC's and the generation of anti-tumor CTL activity. Given this, it is demonstrated that reconstitution of STING signaling in vivo, may help rescue the immunogenic effects of radiation treatment or chemotherapy. Conversely, the data may suggest an additional causative mechanism of autoinflammation involving Ccl531. The data sheds light into the processes of cellular transformation, helps explain why STING signaling is commonly repressed in cancer cells and provides mechanistic insight into how phagocytes can differentiate between non-inflammatory normal apoptotic cells and DNA-damaged or infected cells.

The compounds of this application may be modified by appending various functionalities via any synthetic means delineated herein to enhance selective biological properties. Such modifications are known in the art and include those which increase biological penetration into a given biological system (e.g., blood, lymphatic system, central nervous system), increase oral availability, increase solubility to allow administration by injection, alter metabolism and alter rate of excretion.

### Check Point Inhibitors

The anti-PD-L1 (IgG BE0091, BE0101 or J43 BE0033-2, BioXcell, NH) and anti-PD1 (CD279, BioXcell, NH) were used in the B16 melanoma model.

### Biological Assays

Biological activities of the compounds of the present application can be measured by various biochemical or cellular assays known to one of ordinary skill in the art. Non-limiting examples of biochemical and cellular assays are listed in the Examples vide infra.

### Pharmaceutical Compositions

In another aspect, a pharmaceutical composition is provided. The pharmaceutical composition comprises a therapeutically effective amount of a compound of the application, or a pharmaceutically acceptable salt or ester thereof, and a pharmaceutically acceptable carrier. [0211] Compounds of the application may be administered as pharmaceutical compositions by any conventional route, in particular internally, e.g., orally, e.g., in the form of tablets or capsules, or parenterally, e.g., in the form of injectable solutions or suspensions, or topically, e.g., in the form of lotions, gels, ointments or creams, or in a nasal or suppository form.

### EXAMPLES

### Example 1: Materials, Models and Culture Cell Culture

E.G7 T lymphoblast, C1498 mouse acute myeloid leukemia and mouse melanoma and A375 melanoma cells were obtained from ATCC. B16-OVA cells were kindly provided by Dr. Eli Gilboa (University of Miami). B16-OVA, C1498 and A375 cells were cultured in DMEM supplemented with 10% fetal bovine serum, 4 mM L-glutamine, and 1 mM sodium pyruvate.

### Primary Cell Culture

MEFs were obtained from embryonic day 15 (E15) embryos by a standard procedure as described (Ishikawa and Barber, 2008). The primary macrophages and dendritic cells were isolated from bone marrow of 8-10-week-old mice. Mice were anesthetized with CO₂ and dispatched by cervical dislocation. The bones of the hind legs were dissected, and tissues completely removed. Ends of the bones were cut off and bone marrow were extracted using syringe with 23 G needle. Extracted bone marrow was resuspended with pipet and then washed out through 40 μm cell strainers. To differentiate macrophages, cells derived from bone marrow were incubated with DMEM medium including 20% fetal bovine serum, 1% Penicillin/Streptomycin and 20 ng/ml M-CSF for 7 days in 37° C., 5% CO₂-humidified atmosphere. To differentiate Dendritic cells, 14 days.

### Mice

WT, CCL5 KO and CCR5 KO CL57/B6J mice were purchased from the Jackson Laboratory. SKO mice (Sting−/−) were generated. B6 background WT and SKO were generated from original clone of B6/129 background SKO mice. The original SKO mice in 129/B6 mixed background were backcrossed onto a WT B16 background more than seven times until the hetero mice (Sting+/−) were >97% B16 conventional mice. cGAS KO mice were kindly provided by Dr. Herbert W. Virgin IV (Washington University School of Medicine).

### Mouse Treatment

CL57/B6 WT, Sting KO and cGAS KO mice were maintained in the institutional Division of Veterinary Resources (DVR). All experiments were performed with institutional animal care and use committee (IACUC) approval and in compliance with IACUC guidelines. Tumor cells were injected in the flanks of CL57/B6 mice by subcutaneous injection of 5×10⁵ of the appropriate tumor cells and tumors allowed to develop to an average diameter of 0.5 cm. When tumor reached 100 mm³ in volume, they were exposed to 8 Gy of X-rays at a dose rate of 4.17 Gy/min every other day for a total of three times. Only the tumor was irradiated; the rest of the body was shielded by lead. 10 μg cyclic guanosine monophosphate-adenosine monophosphate (cGAMP) (InvivoGen #tlrl-nacga23) was then intratumorally introduced into the tumors every 2 days for a total of three times. PBS was used as vehicle control. Tumor size was measured using calipers every other day. Tumor volume (V) was calculated using the following formula: V=L*S2/2 (where L is the long axis diameter and S is the short axis diameter).

WT, Sting KO and cGAS KO B16-OVA tumor cells (5×10⁵ cells/mouse) were injected into right flank of WT CL57/B6 mice that purchased from the Jackson laboratory. 7 days after injection, the tumor was exposed to 8 Gy of X-ray radiation every other day for a total of three times followed by tumor bearing mice were kept for 5 days after last X-ray radiation. WT B16-OVA cells (5×10⁵ cells/mouse) were injected to other side flanks for abscopal study and then monitored change of tumor volume every single day.

C1498 or C1498-cGAS AML cells (1×10⁶ cells/mouse) were injected in the flanks of WT CL57/BL6 mice by s.c. injection. Radiation with 8 Gy x-rays was treated after 7 days of tumor inoculation for 3 times every single day. 5-Azadc (Sigma Aldrich #A3656) was introduced by i.p. injection when the tumor diameter reached 100 mm3 in volume for 5 times every 2 days. PBS was used as vehicle control. 2 days after the second injection of 5-Azadc, Tumor was treated to 8 Gy of X-rays every other day for a total of three times. Mice was euthanized 2 days after the last injection of 5-Azadc. To check the abscopal effect by radiation and 5-azadc, 4 days after last 5′-Azadc treatment, 1×10⁶ cells of C1498 cells were subcutaneously injected into the other flank.

E.G7 or E.G7-cGAS cells (5×10⁵ cells/mouse) were subcutaneously inoculated into the flanks of WT CL57/BL6 mice and then exposed with 8 Gy X-rays from 7 days after inoculation of tumor every other day for a total of three times.

### Example 2: Immunoblotting

Cells are lysis with RIPA buffer supplemented with protease inhibitor, phosphatase inhibitor, sodium orthovanadate and PMSF (SantaCruz Biotechnology) and then centrifuged at 4° C., 13000 rpm for 15 mins. Equal amounts of proteins were resolved on SDS-polyacrylamide gels and transferred to polyvinylidene fluoride (PVDF) membrane (Thermo Scientific #88518). After blocking with 5% blocking reagent, membranes were incubated with appropriate primary antibodies. After incubation of primary antibodies at 4C for O/N, membranes were washed 3 times with PBS containing 0.1% Tween 20 for 10 mins and then incubated with secondary antibodies at R/T for 1 h. The image was resolved using an enhanced chemiluminescence system ECL (Thermo Scientific #32106) and detected by autoradiography.

### Example 3: Real Time qPCR

Total RNA was extracted from cell using Trizol (Thermo Scientific #15596026) method and reverse transcribed by High-Capacity cDNA reverse Transcription kit (Applied Biosystems #4368814). Real time qPCR was performed with the TaqMan gene Expression Assay.

### Example 4: Agarose Gel Electrophoresis

2% agarose gel was prepared by melting with TAE buffer and then added with SYBR-Gold (SYBR gold nucleic acid gel staining 10,000× concentration, Thermo Scientific). The agarose/buffer mixture was placed in the gel tray and allowed to solidify for 30 mins. DNA with 6× loading buffer was loaded into well and then ran at 100 v for 90 mins.

### Example 5: In Vitro Phagocytosis

Macrophages were washed with 0.02% EDTA/PBS twice followed by incubation with 0.02% EDTA/PBS at 37C incubator for 10 mins. Cells were collected by scrapping and then centrifuged at 1300 rpm for 5 mins. After centrifugation, BMDM cells (2×10⁵ cells) were plated on 12 well plates and incubated for 48 h. UV or Radiation-treated cells were collected and centrifuged at 2000 rpm for 10 mins. Cells were washed with DMEM media without Fetal Bovine Serum followed by centrifuge at 13000 rpm for 5 mins and the step, which wash, and centrifugation repeated for 3 times. Suspended cells (2×10⁶ cells) with 100 μL DMEM were added to macrophages and then incubated for 6 h. To check efficiency, macrophages were collected at 6 h after phagocytosis of B16 or MEFs stained with CFSE. Cells were washed with PBS, stained with anti-CD11b mAb (BioLegend). Percentage of phagocytosed cells were assessed by flow cytometry using a LSRII instrument (Becton Dickinson, USA).

6×10⁶ BMDCs were plated into 10 cm cell culture dishes and co cultured with 6×10⁶ MEF or B16 cells in the presence of fresh GM-CSF for 6 h. The CD11c positive dendritic cells were purified with Ultrapure CD11c+ positive selection kit (Miltenyi Biotec)

### Example 6: siRNA transfection

B16 and MEF 1×10⁶ cells were plated on 100 mm dish before transfection of siRNA. After 24 h of incubation, 100 nM siRNA transfected with Lipofectamine RNAiMax following manufactural protocol. Cells were incubated for 48 h, and then medium changed to fresh medium.

### Example 7: IFNγ ELISPOT Assay

For tumor-specific CD8+ T cells functional assay, CD8+ T cells were isolated from spleens by using magnetic bead (Miltenyi Biotec #130-104-075). 2×10⁵ CD8+ T cells were stimulated with 1 μg/ml of SIINFEKL peptide for 3 days. After stimulation of SIINFEKL peptide for 2 days, cells were treated with 0.5 μg/ml ionomycin and 50 ng/ml PMA as positive control. ELISPOT assays were performed to detect the cytokine spots for IFNγ following to manufactural protocol (R&D #EL485).

### Example 8: Immunofluorescence

Cells were fixed with 4% paraformaldehyde for 15 mins at 37C and were permeabilized with 0.2% Triton X-100. Fixed and permeabilized cells were blocked with 1% BSA in PBS-T. After blocking, cells were incubated with primary antibodies in 1% BSA in PBS for O/N at 4 C and then washed with PBS-T for 3 times followed by incubation with fluorophore-conjugated secondary antibodies and DPAI counterstaining for 1 h. After staining, cells were mounted in anti-fade mounting solution (Invitrogen) and examined under Leica SP5 Spectral confocal inverted microscope.

### Example 9: Stable Cell Line

C1498 and E.G7 (2×10⁶ cells) cells transfected with pcDNA3-cGAS or pcDNA3-empty plasmid that purchased from Addgene (#102607) using electroporation (Lonza, Nucleofector™ 2b Device) with Nucleofector Kit L (Lonza #VCA 1005). B16 WT. SKO and CKO cells were transfected with pcMV3 empty plasmid or mouse CCL5/RANTES plasmid using Lipofectamine 2000. A375 cells were infected with retrovirus expressing cGAS derived from pBABE-cGAS and then incubated for 1 day followed by washed and incubated for a day. 2 Days after transfection or infection, cells were selected with 1 mg/ml hygromycin B (Thermo Scientific #10687010) or 1 μg/ml Puromycine (Thermo scientific #A1113803) for 3 weeks.

### Example 10: CRISPR Cell Line

2×10⁵ cells of B16 cells were infected with lenti-cas9 for 2 days. After 2 days, cells were selected with 1 μg/m puromycin for 2 weeks and then checked the cas9-positive cells with cas9 antibody. Cas9 expressing B16 cells (2×10⁴ cells) were transfected with 25 nM crRNA and 25 nM tracrRNA using RNAiMAX and then incubated for 3 days. 3 days after transfection, cells were diluted into 96 well plate to select single colonies that were knocked out specific proteins.

### Example 11: Isolation of SIEVE's

Cells (3×10⁶ cells/plate) were plated into 150 mm cell culture dishes overnight, and then treated 50 Gy irradiation followed by incubation for 3 days. Radiation-treated cells were washed and collected with PBS and trypsin-EDTA, respectively. After collection of cells, cells were centrifuged at 2,000 rpm for 10 mins and then re-suspended with 1 ml PBS followed by counting the cell number. The counted cells were centrifuged at 13,000 rpm for 5 mins, and then re-suspended with 1 ml homogenizing buffer [10 mM HEPES (pH 7.3), 1.5 mM MgCl2, 10 mM KC1 and 250 mM sucrose] for lysed cells with homogenizer. Lysates were centrifuged at 300×g for 10 mins and then collection of supernatants to new Eppendorf tube followed by centrifugation at 200×g for 20 min. The supernatant was transferred to a new tube and centrifuged at 10,000×g for 1 h. The supernatant was transferred to ultracentrifuge tube (25×89 mm, Beckman) and ultracentrifuged at 100,000×g for 90 mins. The pellet was washed by centrifuging at 100,000×g for 90 mins. The final pellet was re-suspended with 100 μl PBS and stored at 4 C.

### Example 12: Measurement of DNA With Picogreen

Picogreen (Picogreen dsDNA assay kit, Invitrogen) solution was prepared by diluting 200-fold into 1×TE in plastic tubes and then dispensed 100 μL working solution of Picogreen to each well with pipetting up and down for 5 times. The plate was covered with foil and incubated at room temperature for 5 mins. After 5 mins, the plate was read with CLARIO STARPLUS (BMG LABTECH). Lambda DNA was diluted to 2 μg/mL in 1×TE and used for standard curve.

### Example 13: DNA Isolation From SIEVE's

DNA from vesicles was extracted with QIAamp DNA mini kit (QIAGEN) by following manufacturer's instructions. Briefly, 400 μL lysed vesicles including 20 μL QIAGEN protease, 100 μL PBS and 200 μL AL buffer, were mixed by vortexing for 15 secs and then incubated at 56° C. for 10 mins. 200 μL ethanol was added into the mixture and mixed by vortexing for 15 secs. The mixture was transferred to QIAamp column and then centrifuged at 13,000 rpm for 1 min. After centrifugation, column was washed with 750 μL AW1 and AW2, and then eluted with 100 μL distilled water. The eluted DNA concentration was measured with Nanodrop (Nanodrop One, Thermo Scientific) and Picogreen staining.

### Example 14: 2′3′ cGAMP ELISA

B16-OVA cells (WT, Sting KO and cGAS KO B16-OVA), C1498, A375-cGAS, E.G7, E.G7-cGAS, A375 and A375-cGAS cells were plated in 100 mm dish and then incubated for 24 h at 37° C. incubator. Cells were irradiated with 50 Gy and 20 Gy in B16-OVA cell, C1498 and EL4 cells, respectively, followed by incubation for 1 to 6 days. To lyse the cells, cells were lysed with M-PERTM Mammalian protein extraction reagent (Thermo Scientific #78503) and incubated on the ice for 15 mins, after incubation, cells were sonicated at a frequency of 20 kHz for 3×10 sec. Lysed cells were centrifuged at 4° C., 13000 rpm for 15 mins. ELISA is followed by manufactural protocol (Cayman Chemical #501700).

### Example 15: Immunohistochemistry

Mice were sacrificed, and the tumor biopsies were fixed in 10% formaldehyde for 72 h. All processes for paraffin block were performed at the Pathology Research Resource Histology Laboratory in University of Miami. Immunohistochemistry (IHC). Staining was performed with appropriated antibody following to standard protocol.

### Example 16: Microarray

Total RNA was isolated from cells with RNeasy Mini kit (Qiagen). RNA quality was analyzed by Bioanalyzer RNA 6000 Nano (Agilent Technologies). Gene array analysis was examined by Affymetrix Mouse Gene array (2.0 ST Array) at the Center for Genome Technology, John P. Hussman Institute for Human Genomics, University of Miami. Gene expression profiles and statistical analysis was performed by Biostatistics & Bioinformatics Shared Resource at Sylvester Comprehensive Cancer Center, University of Miami.

### Example 17: ChIP Assay

Cells were transfected with ISD and further cross-linked with 1% formaldehyde at 37° C. for 10 mins and then incubated with glycine to terminate reactions at room temperature for 5 min. Cells were washed with PBS, lysed, and incubated with CCL5/RANTES antibody coated plate at room temperature for 2 h. The bound protein was washed with PBS-T five times followed by incubation with 5 μL of 5 M NaCl and 2 μL RNase A (20 mg/ml) in PBS at 65° C. overnight. After incubation, de-cross-linked mixtures were added with 4 μL of 0.5M EDTA, 8 μL of 1M Tris-HCl (pH6.5) and 1 μL proteinase K (20 mg/ml) at 45° C. for 1 h. Nucleic acid were isolated with phenol-chloroform, precipitated with ethanol, and analyzed by qPCR. GAPDH was used as a reference.

### Example 18: Binding of CCL5 to DNA

ISD90 was premixed with CCL5 and negative control molecules at different concentration in 100 μL and analyzed by a fluorimeter after staining with Picogreen.

### Example 19: Electrophoretic Mobility Shift Assays (EMSA)

ISD90 (10 μg/ml) was premixed by mixing various concentrations of CCL5 at 37C for 15 mins and then the samples were run on 2% agarose gel in 1×TBE for 1 h, stained with SYBR-Gold, and visualized under UV light.

### Example 20: Nanoparticle Tracking Analysis (NTA)

The isolated SIEVEs were diluted with endotoxin free PBS and labelled with CD81-Dylight550 antibody (Novus Biologicals #NB100-65805R). NTA and fluorescent NTA were conducted by Alpha Nano Tech, LLC (Morrisveill, NC, USA) on the Zetaview Quatt (Particle Metrix).

### Example 21: Proteomics Analysis

Proteomics analysis of proteins were conducted by Alpha Nano Tech, LLC (Morrisveill, NC, USA) using LC-MS/MS with a Thermo Easy nLC 1200-QEactive HF. Raw data were analyzed using proteome Discoverer 2.5-searched against the Uniprot mouse database (containing ˜17,000 protein sequences) and a common contaminants database (containing ˜245 sequences). Further analysis and statistics (Log2 transformation, imputation, t-test) were conducted by Perseus.

### Example 22: Dot Blot Assay

DNA isolated SIEVE were desaturated with 0.1 N NaOH at 95° C. for 5 mins followed by neutralizing the DNA with 1 M ammonium acetate on ice. The DNA was spotted on nitrocellulose membrane and incubated at 80° C. for 30 mins and then cross-linked with UV (125 mJ/cm²). The membrane was blocked with 5% skim milk in PBS containing 0.1 Tween-20 for 1 h followed by incubating with antibody against dsDNA at 4° C. for O/N. After incubation with primary antibody, the membrane was washed with PBS-T 3 times and then incubated with HRP-conjugated mouse secondary antibody at room temperature for 1 h. The blots were visualized using chemiluminescence kit according to the manufacturer's instructions.

### Example 23: Nuclease Protection Assay of Ccl5-DNA Complex

The suspension containing CCL5-mouse genomic DNA complex was incubated at 37° C. with 1 unit DNase II (Sigma-Aldrich) for 20 mins. The DNA-CCL5 Complexes were run on 2% agarose gel in 1 X TBE stained with SYBR-Gold and visualized under UV light.

### Example 24: Statistical Analysis

All data are representative of multiple independent experiments, and they are presented as the average+SD. Two-tailed Student's t tests were used to evaluate the differences between two groups, where P<0.05 was considered statistically significant.

### Further Embodiments

Embodiments contemplated herein include Embodiments P1-P20 following.

Embodiment P1. A method of treating cancer in a subject in need thereof including administering a radiation therapy to the subject, where the subject has been determined to be responsive to the radiation therapy including contacting a sample from the subject including one or more nucleic acid molecules with a device including a single-stranded nucleic acid molecule capable of specifically hybridizing with nucleotides of a CCL5 gene, and detecting a level of expression of the CCL5 gene by performing microarray analysis or quantitative reverse transcriptase polymerase chain reaction (qRT-PCR) of the sample, and determining that the CCL5 gene is expressed in the subject.

Embodiment P2. The method of Embodiment P1, further including augmenting CCL5 production by administering type I interferon to the subject.

Embodiment P3. The method of Embodiment P1, where the type I interferon is administered in a tumor in the subject.

Embodiment P4. The method of Embodiment P1, where the type I interferon is administered in the presence of the radiation therapy.

Embodiment P5. The method of Embodiment P1, where the type I interferon is administered i.p. into the tumor.

Embodiment P6. The method of Embodiment P1, where further including where type I interferon is expressed in the sample.

Embodiment P7. A method of treating a cancer in a subject in need thereof including contacting a sample from the subject including one or more nucleic acid molecules with a device including single-stranded nucleic acid molecules capable of specifically hybridizing with nucleotides of a CCL5 gene, detecting a level of expression of the CCL5 gene by performing microarray analysis or quantitative reverse transcriptase polymerase chain reaction (qRT-PCR) of the sample, determining that the CCL5 gene is expressed in the subject, and administering a therapy to the subject thereby providing the method of treating the cancer.

Embodiment P8. The method of Embodiment P7, further including augmenting CCL5 production by administering type I interferon to the subject.

Embodiment P9. The method of Embodiment P7, where the type I interferon is administered in a tumor in the subject.

Embodiment P10. The method of Embodiment P7, where the type I interferon is administered in the presence of the radiation therapy.

Embodiment P11. The method of Embodiment P7, where the type I interferon is administered i.p. into the tumor.

Embodiment P12. The method of Embodiment P7, where further including where type I interferon is expressed in the sample.

Embodiment P13. The method of Embodiment P7, where the type I interferon is administered in a tumor in the subject.

Embodiment P14. The method of Embodiment P7, where the therapy is selected from the group consisting of radiation therapy, chemotherapy, immunotherapy and car-T therapy.

Embodiment P15. A method of treating a cancer in a subject in need thereof including contacting a sample from the subject including one or more nucleic acid molecules with a device including single-stranded nucleic acid molecules capable of specifically hybridizing with nucleotides of a CCL5 gene, detecting a level of expression of the CCL5 gene by performing microarray analysis or quantitative reverse transcriptase polymerase chain reaction (qRT-PCR) of the sample, determining that the CCL5 gene is expressed in the subject, and administering a radiation therapy to the subject thereby providing the method of treating the cancer, where the radiation therapy is administered in a dosage between a lower limit of approximately 1 Rad/day, and an upper limit of approximately 100 Rad/day.

Embodiment P7. A method of treating a cancer in a subject in need thereof including contacting a sample from the subject including one or more nucleic acid molecules with a device including single-stranded nucleic acid molecules capable of specifically hybridizing with nucleotides of a CCL5 gene, detecting a level of expression of the CCL5 gene by performing microarray analysis or quantitative reverse transcriptase polymerase chain reaction (qRT-PCR) of the sample, determining that the CCL5 gene is expressed in the subject, and administering a therapy to the subject thereby providing the method of treating the cancer.

Embodiment P8. The method of Embodiment P7, further including augmenting CCL5 production by administering type I interferon to the subject.

Embodiment P9. The method of Embodiment P7, where the type I interferon is administered in a tumor in the subject.

Embodiment P10. The method of Embodiment P7, where the type I interferon is administered in the presence of the radiation therapy.

Embodiment P11. The method of Embodiment P7, where the type I interferon is administered i.p. into the tumor.

Embodiment P12. The method of Embodiment P7, where further including where type I interferon is expressed in the sample.

Embodiment P13. The method of Embodiment P7, where the type I interferon is administered in a tumor in the subject.

Embodiment P14. The method of Embodiment P7, where the therapy is selected from the group consisting of radiation therapy, chemotherapy, immunotherapy and car-T therapy.

Embodiment P15. A method of treating a cancer in a subject in need thereof including contacting a sample from the subject including one or more nucleic acid molecules with a device including single-stranded nucleic acid molecules capable of specifically hybridizing with nucleotides of a CCL5 gene, detecting a level of expression of the CCL5 gene by performing microarray analysis or quantitative reverse transcriptase polymerase chain reaction (qRT-PCR) of the sample, determining that the CCL5 gene is expressed in the subject, and administering a radiation therapy to the subject thereby providing the method of treating the cancer, where the radiation therapy is administered in a dosage between a lower limit of approximately 1 Rad/day, and an upper limit of approximately 100 Rad/day, described for illustrative purposes only, and are not limiting. Other embodiments are possible and are covered by the invention. Such embodiments will be apparent to persons skilled in the relevant art(s) based on the teachings contained herein. For example, it is envisaged that, irrespective of the actual shape depicted in the various Figures and embodiments described above, the outer diameter exit of the inlet tube can be tapered or non-tapered and the outer diameter entrance of the outlet tube can be tapered or non-tapered.

Thus, the breadth and scope of the present invention should not be limited by any of the above-described exemplary embodiments, but should be defined only in accordance with the following claims and their equivalent.

What is claimed is:

1. A method of treating cancer in a subject in need thereof comprising administering a radiation therapy to the subject, where the subject has been determined to be responsive to the radiation therapy comprising:
(a) contacting a sample from the subject comprising one or more nucleic acid molecules with a device comprising a single-stranded nucleic acid molecule capable of specifically hybridizing with nucleotides of a CCL5 gene;
(b) detecting a level of expression of the CCL5 gene by performing microarray analysis or quantitative reverse transcriptase polymerase chain reaction (qRT-PCR) of the sample; and
(c) determining that the CCL5 gene is expressed in the subject.

2. The method of claim 1, further comprising augmenting CCL5 production by administering type I interferon to the subject.

3. The method of claim 2, where the type I interferon is administered in a tumor into the subject.

4. The method of claim 3, where the type I interferon is administered in the presence of the radiation therapy.

5. The method of claim 3, where the type I interferon is administered i.p. into the tumor.

6. The method of claim 1, further comprising where type I interferon is expressed in the sample.

7. A method of treating a cancer in a subject in need thereof comprising:
(a) contacting a sample from the subject comprising one or more nucleic acid molecules with a device comprising single-stranded nucleic acid molecules capable of specifically hybridizing with nucleotides of a CCL5 gene;
(b) detecting a level of expression of the CCL5 gene by performing microarray analysis or quantitative reverse transcriptase polymerase chain reaction (qRT-PCR) of the sample;
(c) determining that the CCL5 gene is expressed in the subject; and
(d) administering a therapy to the subject thereby providing the method of treating the cancer.

8. The method of claim 7, further comprising augmenting CCL5 production by administering type I interferon to the subject.

9. The method of claim 8, where the type I interferon is administered in a tumor into the subject.

10. The method of claim 9, where the type I interferon is administered in the presence of a radiation therapy.

11. The method of claim 9, where the type I interferon is administered i.p. into the tumor.

12. The method of claim 7, further comprising where type I interferon is expressed in the sample.

13. The method of claim 7, where the therapy is selected from the group consisting of radiation therapy, chemotherapy, immunotherapy and car-T therapy.

14. A method of treating a cancer in a subject in need thereof comprising:
(a) contacting a sample from the subject comprising one or more nucleic acid molecules with a device comprising single-stranded nucleic acid molecules capable of specifically hybridizing with nucleotides of a CCL5 gene;
(b) detecting a level of expression of the CCL5 gene by performing microarray analysis or quantitative reverse transcriptase polymerase chain reaction (qRT-PCR) of the sample;
(c) determining that the CCL5 gene is expressed in the subject; and
(d) administering a radiation therapy to the subject thereby providing the method of treating the cancer, where the radiation therapy is administered in a dosage between:
a lower limit of approximately 1 Rad/day; and
an upper limit of approximately 100 Rad/day.

15. The method of claim 14, further comprising augmenting CCL5 production by administering type I interferon to the subject.

16. The method of claim 15, where the type I interferon is administered in a tumor in to the subject.

17. The method of claim 15, where the type I interferon is administered in the presence of the radiation therapy.

18. The method of claim 15, where the type I interferon is administered i.p. into a tumor.

19. The method of claim 14, further comprising where type I interferon is expressed in the sample.
